SEQUENCE LISTING (16 sequences)

SEQ ID NO: 1 - DNA, 76 bp, Artificial
  Shows the cloverleaf structures of amber and ochre suppressor tRNAs derived from E. coli initiator tRNA.
cgcggggsggagcagccuggdagcucgucgggcucauaacccgaagaucgucggtcaaagccggcccccgcaacca

SEQ ID NO: 2 - DNA, 83 bp, Artificial
  Shows the supF amber suppressor tRNA derived from E. coli tyrosine tRNA.
gguggggsscccgagcggccaaagggagcagacuauaaacugccgucaucgacuucgaaggtcgaauccuucccccacgacca

SEQ ID NO: 3 - DNA, 43 bp, Artificial
  forward primer
ggggccatggaccaatttgttggggtatagccaagcggtaagg

SEQ ID NO: 4 - DNA, 36 bp, Artificial
  reverse primer
ggggtacgtattgaataaattggctggggtacgagg

SEQ ID NO: 5 - DNA, 52 bp, Artificial
  forward primer
cccgaattcgccaccatgcatcaccatcaccatcacagtgaggcagaagccc

SEQ ID NO: 6 - DNA, 31 bp, Artificial
  reverse primer
cccgcggccgcttactcgcctactttcgccc

SEQ ID NO: 7 - DNA, 52 bp, Artificial
  Amber suppressor tRNAs
ugggguauagccaagcgguaaggcaccggauucuaauuccggcauuccgagg

SEQ ID NO: 8 - DNA, 72 bp, Artificial
  Amber suppressor tRNAs
ugggguauagccaagcgguaaggcaccggacucuaauuccggcauuccgagguucgaauccucguaccccag

SEQ ID NO: 9 - DNA, 72 bp, Artificial
  Amber suppressor tRNAs
ugggguauagccaagcgguaaggcaccggauucuaaauccggcauuccgagguucgaauccucguaccccag

SEQ ID NO: 10 - DNA, 72 bp, Artificial
  Amber suppressor tRNAs
ugggguauagccaagcgguaaggcaccggacucuaaauccggcauuccgagguucgaauccucguaccccag

SEQ ID NO: 11 - DNA, 72 bp, Artificial
  Amber suppressor tRNAs
ugggguauagccaagcgguaaggcaccggauuuuaauuccggcauuccgagguucgaauccucguaccccag

SEQ ID NO: 12 - DNA, 72 bp, Artificial
  Amber suppressor tRNAs
ugggguauagccaagcgguaaggcaccggacuuuaauuccggcauuccgagguucgaauccucguaccccag

SEQ ID NO: 13 - DNA, 56 bp, Artificial
  Amber suppressor tRNAs
ggggaagccaagcggaaggcaccggaaaaccggcaccgaggcgaacccgaccccag

SEQ ID NO: 14 - DNA, 72 bp, Artificial
  Amber suppressor tRNAs
ugggguauagccaagcgguaaggcaccggacuuuaaauccggcauuccgagguucgaauccucguaccccag

SEQ ID NO: 15 - DNA, 72 bp, Artificial
  Amber suppressor tRNAs
ugggguauagccaagcgguaaggcaccggauuucaauuccggcauuccgagguucgaauccucguaccccag

SEQ ID NO: 16 - DNA, 72 bp, Artificial
  Amber suppressor tRNAs
ugggguauagccaagcgguaaggcaccggacuucaaauccggcauuccgagguucgaauccucguaccccag

(19) United States
(12) Patent Application Publication
(10) Pub. No.: US 2006/0134748 A1
(43) Pub. Date: Jun. 22, 2006

(54) Orthogonal suppressor tRNAs and aminoacyl-tRNA synthetases and uses thereof

(76) Inventors: Uttam RajBhandary, Lexington, MA (US); Caroline Koehrer, Somerville, MA (US)

Correspondence Address: CHOATE, HALL & STEWART LLP, TWO INTERNATIONAL PLACE, BOSTON, MA, 02110, US

(21) Appl. No.: 11/284,259

(22) Filed: Nov. 21, 2005

Related U.S. Application Data
(60) Provisional application No. 60/629,776, filed on Nov. 20, 2004.

(51) Int. Cl. C12P 21/06; C12N 9/22; C12N 15/87

(52) U.S. Cl. 435/69.1; 435/455; 435/325; 530/350; 435/199

(57) ABSTRACT

The present invention provides novel orthogonal suppressor tRNAs, aminoacyl-tRNA synthetases, and suppressor tRNA/aminoacyl-tRNA synthetase pairs. In preferred embodiments the suppressor tRNAs function in mammalian cells and in certain embodiments they are not aminoacylated by any of the mammalian cytoplasmic aminoacyl-tRNA synthetases. The invention provides a novel ochre suppressor tRNA that fulfills these criteria. The invention further provides novel amber and opal suppressor tRNAs having a range of different translation efficiencies. The invention also provides mammalian cells containing the suppressor tRNAs, aminoacyl-tRNA synthetases, or both. The suppressor tRNAs may be introduced into the cell from the exterior or may be expressed by the cell. They may be aminoacylated with either a natural or an unnatural amino acid. The suppressor tRNAs, aminoacyl-tRNA synthetases, or both, may be expressed by a mammalian cell in a regulated manner. The invention further provides methods for synthesizing proteins using the inventive suppressor tRNAs and aminoacyl-tRNA synthetases and proteins produced by the methods.

### CROSS-REFERENCE TO RELATED APPLICATION

This application claims priority to U.S. Provisional Patent Application 60/629,776, filed Nov. 20, 2004, which is incorporated herein by reference.

### BACKGROUND OF THE INVENTION

Significant research effort has been directed toward the development of techniques to introduce unnatural amino acids into polypeptide chains, either by chemical synthesis (see, for example, Hofman et al., J. Am. Chem. Soc. 88:5914, 1966), semi-synthetic approaches (see, for example, Borras et al., Nature 227:716, 1970; Sealock et al., Biochemistry 8:3703, 1969; Inouye et al., J. Am. Chem. Soc. 101:752, 1979), modification of reactive side-groups in extant polypeptides (see, for example, Neet et al., Proc. Natl. Acad. Sci. USA 56:1606, 1966; Polgar et al., J. Am. Chem. Soc. 88:3153, 1966; Kaiser et al., Science 266:505, 1984; Mayo et al., Science233:948, 1986), or use of alternatively acylated tRNAs (see, for example, Krieg et al., Proc. Natl. Acad. Sci. USA 83:8604, 1986; Wiedmann et al., Nature 328: 830, 1987; Johnson et al., Biochemistry 15:569, 1976; Baldini et al., Biochemistry 27:7951, 1988; Roesser et al., Biochemistry 25:6361, 1986; Heckler et al., J. Biol. Chem. 258:4492, 1983; Noren et al., Science 244:182, 1989; Ellman et al., Met. Enzymol. 202:301, 1991). See also Bain et al., Biochemistry 30:5411-5421, 1991; Bain et al., J. Am. Chem. Soc. 111:8013-8014, 1989; Heckler, et al., Biochemistry, 30:1468-1473. Introduction of such unnatural amino acids into proteins allows analysis of protein folding and/or activity, and also allows adjustment of protein characteristics such as solubility, stability, etc.

Unfortunately, most of the techniques available for introducing unnatural amino acids into proteins generate only low protein yields. Furthermore, many techniques can only be utilized in vitro and/or rely on laborious synthetic technologies. Also, those techniques that utilize alternatively acylated tRNAs can typically introduce only a single unnatural amino acid into a given polypeptide chain. There remains a need for the development of more generally applicable systems for introducing unnatural amino acids into proteins. Preferably, such systems should allow unnatural amino acids to be incorporated into growing polypeptide chains in vivo. Alternatively or additionally, such systems should be able to introduce multiple unnatural amino acids into a single protein.

Most of the work on unnatural amino acid mutagenesis has involved the use of an amber suppressor transfer RNA (tRNA) along with an amber stop codon at the site of interest in the protein gene. The availability of other suppressor tRNA/stop codon pairs would greatly add to the versatility of unnatural amino acid mutagenesis and allow site-specific insertion of two or more different unnatural amino acids into proteins. In particular, for suppression of stop codons and site-specific insertion of unnatural amino acids in mammalian translation systems, e.g., mammalian cells, there is a need in the art for suppressor tRNAs that do not serve as substrates for aminoacyl-tRNA synthetases in mammalian cells. There is likewise a need in the art for aminoacyl-tRNA synthetases that aminoacylate such suppressor tRNAs but do not significantly aminoacylate mammalian tRNAs.

Significant research effort has been directed at developing techniques and reagents for the treatment or cure of various human genetic diseases. There remains a need for the development of improved systems.

### SUMMARY OF THE INVENTION

The present invention provides methods and reagents for reading through stop codons in mammalian cells. In particular, the invention allows suppressor tRNAs that are generated outside of mammalian cells to be introduced into those cells, where they suppress nonsense mutations. In certain embodiments of the invention, the suppressor tRNAs are aminoacylated prior to introduction into the mammalian cells; in other embodiments, they are not aminoacylated prior to introduction. In certain embodiments of the invention the tRNAs are expressed in mammalian cells. In some preferred embodiments, the tRNAs utilized are not substrates for aminoacyl tRNA synthetases present within the cell. In general, however, when tRNAs are not aminoacylated prior to import into cells, it is preferred that the tRNAs are substrates for endogenous tRNA synthetase(s). The endogenous aminoacyl tRNA synthetase(s) may be native to the cell or may be non-native.

The techniques and reagents of the present invention may be utilized to introduce one or more unnatural amino acids into polypeptides synthesized in mammalian cells; in certain embodiments such polypeptides contain at least two or more unnatural amino acids. For example, in certain embodiments such polypeptides contain two different unnatural amino acids; in other embodiments such polypeptides contain three different unnatural amino acids. Alternatively or additionally, inventive methods and/or reagents may be utilized to read through stop codons responsible for a disease phenotype in a mammalian cell.

Inventive methods and/or reagents may also be used to maintain mammalian cells containing nonsense mutations in one or more genes in culture. Mammalian cells containing inventive suppressor tRNAs and aminoacyl-tRNA synthetases that aminoacylate such tRNAs can also be used for the isolation and propagation of viruses that contain nonsense mutations in one or more viral genes. The invention thus provides a system for the isolation and propagation of a mutant animal virus.

The invention also provides methods and reagents for synthesizing proteins containing one, two, or more unnatural amino acids in vitro, e.g., in a mammalian in vitro translation system, by readthrough of one, two, or three different stop codons.

In one aspect, the invention provides novel suppressor tRNAs that are not substrates for any native aminoacyl-tRNA synthetases when introduced into or expressed in mammalian cells. To the best of the inventors' knowledge, the novel suppressor tRNAs include the first example of an ochre suppressor that is not significantly aminoacylated by mammalian aminoacyl-tRNA synthetases.

In another aspect, the invention provides a complete set of suppressor tRNAs (ochre, amber, and opal) that are not substrates for any native aminoacyl-tRNA synthetase when introduced into or expressed in mammalian cells. The invention further provides an aminoacyl-tRNA synthetase that aminoacylates these suppressor tRNAs but does not aminoacylate native tRNAs in a mammalian cell. As is known to one of ordinary skill in the art, such tRNAs, aminoacyl-tRNA synthetases, and pairs thereof are referred to as orthogonal. To the best of the inventors' knowledge, the invention thus provides the first complete set of orthogonal aminoacyl-tRNA synthetase-amber suppressor tRNA, aminoacyl-tRNA synthetase-ochre suppressor tRNA, and aminoacyl-tRNA synthetase-opal suppressor tRNA pairs for use in mammalian translation systems, e.g., mammalian cells. Because most cells contain 20 aminoacyl-tRNA synthetases (aaRSs), the orthogonal synthetase-suppressor tRNA pairs are often called 21^{st} synthetase-tRNA pairs.

In another aspect, the invention provides orthogonal amber suppressor tRNAs, ochre, and opal suppressor tRNAs, and collections or “sets” thereof, having a wide range of different suppressor activities in mammalian cells, e.g., a collection of ochre suppressor tRNAs, a collection of amber suppressor tRNAs, a collection of opal suppressor tRNAs, or combinations thereof. Certain of the amber, ochre, and opal suppressor tRNAs display high translation efficiencies in mammalian cells, e.g., on the same order as those of homologous human serine amber, ochre and opal suppressor tRNAs.

The invention further provides kits comprising one or more of amber, ochre, and/or opal suppressor tRNAs of collections thereof. The kits may also include, for example, an aaRS that aminoacylates one or more of the suppressor tRNAs. The aaRS may be, for example, a bacterial glutaminyl-tRNA synthetase (GlnRS, QRS), or a bacterial tryptophanyl-tRNA synthetase (TrpRS, WRS).

The suppressor tRNAs may be imported into a mammalian cell or may be expressed in a mammalian cell. The mammalian cell may contain an aminoacyl-tRNA synthetase (aaRS) that aminoacylates one or more of the suppressor tRNAs. Preferably the aaRS does not significantly aminoacylate any native tRNA in a mammalian cell. The aaRS may be expressed in the cell in a regulatable manner, e.g., under control of an inducible or repressible promoter. The aaRS may be, for example, a bacterial glutaminyl-tRNA synthetase (GlnRS, QRS), or a bacterial tryptophanyl-tRNA synthetase (TrpRS, WRS).

Activity of the suppressor tRNAs may be regulatable, e.g., by regulating expression or activity of an aaRS that aminoacylates them or by regulating expression of the suppressor tRNA. In one embodiment, a nucleic acid construct comprising a polynucleotide sequence that encodes the suppressor tRNA or the aaRS, operably linked to an inducible or repressible promoter, can be introduced into mammalian cells. The expression of the suppressor tRNA or aaRS is regulated by exposing the cell to appropriate conditions to induce or repress the promoter. For example, the cell can be contacted with an agent that induces or represses the promoter.

Methods and accompanying reagents, e.g., reporter systems, for testing the inventive compositions are also provided. The reporter systems may be used for the development and testing of additional suppressor tRNAs and aminoacyl-tRNA synthetases having desired features, e.g., additional tRNAs that are not aminoacylated by mammalian aminoacyl-tRNA synthetases, additional aminoacyl-tRNA synthetases that aminoacylate such tRNAs, and aminoacyl-tRNA synthetases that do not aminoacylate tRNAs present within a system of interest, e.g., the mammalian cell cytoplasm.

Although the invention is described largely in reference to mammalian translation systems, e.g., mammalian cells, the use of the inventive suppressor tRNAs is not limited to mammalian cells but extends also to other animal cells or organisms, (e.g., insect, Xenopus oocyte, etc.), plant, fungi such as yeast, other eukaryotes, and also prokaryotic species).

Unless otherwise stated, the invention makes use of standard methods of molecular biology, cell culture, etc., and uses art-accepted meanings of terms. This application refers to various patents and publications. The contents of all of these are incorporated by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001. The contents of U.S.S.N. 10/271,453, filed Oct. 16, 2003, and U.S. Provisional Patent Application 60/329,702, filed Oct. 16, 2001, are also incorporated herein by reference. In the event of a conflict between any of the incorporated references and the instant specification, the specification shall control. The determination of whether a conflict or inconsistency exists is within the discretion of the inventors and can be made at any time.

### BRIEF DESCRIPTION OF THE DRAWING

The invention is described with reference to the several Figures of the drawing, in which,

FIG. 1 presents a scheme for assaying import and function of amber suppressor tRNA.

FIG. 2A (SEQ ID NO: 1) shows the cloverleaf structures of amber and ochre suppressor tRNAs derived from E. coli initiator tRNA^{fMet}. The ochre suppressor contains the U34 mutation (in parenthesis) in addition to the other mutations present in the amber suppressor tRNA. FIG. 2B (SEQ ID NO: 2) shows the supF amber suppressor tRNA derived from E. coli tyrosine tRNA. Arrows indicate the sequence changes in the suppressor tRNAs.

FIG. 3A shows CAT activity detected in extracts of cells co-transfected with the pRSVCATam27 DNA and varying amounts of amber suppressor tRNA, with or without aminoacylation. FIG. 3B shows CAT activity detected in extracts of cells co-transfected with the wild type pRSVCAT DNA with or without the amber suppressor tRNA. The CAT activities are the average of three independent experiments. ND=not detectable.

FIG. 4 shows acid urea gel analysis of tRNA isolated from cells co-transfected with pRSVCATam27 DNA and increasing amounts of the amber suppressor tRNA derived from the E. coli tRNA^{fMet} (lanes 1-5). Lane 5 contains the same sample as lane 4 except that the aminoacyl linkage to the tRNA was hydrolyzed by base treatment (OH⁻). Lanes 6 and 7 provide markers for tRNA and Tyr-tRNA, respectively.

FIG. 5 shows results of thin layer chromatographic assay for CAT activity in extracts of COS1 cells transfected with pRSVCATam27 DNA (lanes 1 and 4) and supF tRNA, uncharged (lanes 2 and 3), or charged (lanes 5 and 6). Lane 7, mock transfected; CAM, unreacted substrate and Ac-CAM, the products formed. The CAT activities are the average of two independent experiments. ND, not detectable.

FIG. 6 presents illustrative examples of certain unnatural amino acids that could be incorporated into a protein or polypeptide in accordance with the present invention. FIG. 6A shows certain fluorescent amino acid analogs; FIG. 6B shows an amino acid analog including a heavy atom label (I, which is useful, for instance, in X-ray crystallography; analogs containing F rather than I could be used, for example, for NMR spectroscopy); FIG. 6C shows certain amino acid analogs that include reactive moieties such as photoactivatable groups useful for cross-linking; FIG. 6D depicts a phosphotyrosine analog useful in the practice of the present invention, for example to facilitate the study of cell signalling.

FIG. 7A shows a scheme for import of aminoacylated suppressor tRNAs for concomitant suppression of amber and ochre codons in a single mRNA. FIG. 7B is a schematic representation of the luciferase reporter mRNA encoding a Renilla luciferase/firefly luciferase (RLucFLuc) fusion protein. Top, RLucFLuc (am7O) or RLucFLuc (oc70); bottom, RLucFLuc (oc70/am165). Stop mutations in the firefly luciferase gene are indicated.

FIG. 8 shows the cloverleaf structures of the suppressor tRNAs derived from the E. coli tyrosine tRNA. (A) supF amber suppressor tRNA; (B) supC.A32 ochre suppressor tRNA. Arrows indicate the changes in the suppressor tRNAs.

FIG. 9 shows acid urea gel analysis of suppressor tRNAs before and after in vitro aminoacylation with tyrosine. Lanes 1 and 2, supF amber suppressor tRNA; lanes 3 and 4, supC.A32 ochre suppressor tRNA. Suppressor tRNAs were visualized by Northern hybridization using radiolabeled oligonucleotides specific for the anticodon stem-loop regions of supF and supC.A32 tRNA, respectively.

FIG. 10 shows cloverleaf structures of suppressor tRNAs derived from E. coli tRNA^{Gln}. The mutated anticodon sequences and the C9 to A9 mutation are circled.

FIG. 11 shows a schematic representation of the luciferase reporter mRNA encoding a Renilla luciferase-firefly luciferase (RLucFLuc) fusion protein. Internal stop codon mutations in the firefly luciferase gene are indicated. The luciferase reporter mRNA has two termination signals at the 3′-terminus separated by a UUC codon (. . . UAAUUCUAG . . . polyA . . . ; termination codons are underlined).

FIG. 12 shows acid urea PAGE/Northern blot analysis of hsup2am, hsup2oc and hsup2op tRNAs. Total tRNA was isolated under acidic conditions and separated by acid urea PAGE. Suppressor tRNAs were visualized by RNA blot hybridization using a 5-³²P-labeled oligonucleotide complementary to nucleotides 57-72 of E. coli tRNA^{Gln}. A 5-³²P-labeled oligonucleotide complementary to nucleotides 7-22 of the human tRNA^{Ser} was used as internal standard for quantitation of RNA and aminoacylation levels by PhosphorImager analysis.

FIG. 13 shows amber, ochre and opal suppression in HEK293T cells. Immunoblot analysis of proteins isolated from cells co-transfected with plasmids carrying the genes encoding the luciferase reporter, hsup2/C32A38am, hsup2/C32A38oc or the hsup2/C32A38op tRNAs and, when present, E. coli GlnRS. The RLucFLuc fusion protein was detected with an anti-FLuc antibody and E. coli GlnRS was detected with an anti-His4-antibody. An antibody against β-actin was used as a loading control. RLucFLuc, full length fusion protein; RLucFLuc*, truncated RLucFLuc fusion protein.

FIG. 14 shows acid urea PAGE/Northern blot analysis of additional mutants derived from hsup2am, hsup2oc and hsup2op tRNAs. (A) amber suppressor series; (B) ochre suppressor series; (C) opal suppressor series. Suppressor tRNAs were visualized by RNA blot hybridization using a 5′-³²P-labeled oligonucleotide complementary to nucleotides 57-72 of tRNA^{Gln}. A 5-³²P-labeled oligonucleotide complementary to nucleotides 7-22 of the human tRNA^{Ser} was used as internal standard (data not shown) for quantitation of RNA and aminoacylation levels by PhosphorImager analysis.

FIG. 15 shows β-galactosidase activity in cell extracts of E. coli with an amber mutation in the chromosomal β-galactosidase gene transformed with plasmids carrying the hsup2am, hsup2/C32A38am, hsup2oc and hsup2/C32A38oc tRNA genes. Values represent the averages of at least three independent experiments.

FIG. 16 shows the cloverleaf structures of E. coli tRNA^{Gln} (A), E. coli tRNA^{Trp} (B) and hsup2/C32A38 suppressor tRNAs (C).

FIG. 17 shows firefly luciferase activity in cell extracts of HEK293T cells transfected with plasmids carrying the genes for hsup2/C32A38am tRNA (A), hsup2/C32A38oc tRNA (B), and hsup2/C32A38op tRNA (C) and E. coli GlnRS (QRS) or E. coli TrpRS (WRS) as indicated. Cells were also co-transfected with a plasmid encoding the reporter RLucFLuc fusion protein with the appropriate amber, ochre or opal mutation to measure suppression activity. Luciferase activities are given as relative luminescence units (RLU) per μg of total cell protein.

FIG. 18 is an immunoblot showing E. coli GlnRS and E. coli TrpRS-dependent amber, ochre and opal suppression in HEK293T cells. Immunoblot analysis of proteins isolated from cells co-transfected with plasmids carrying the genes encoding the luciferase reporter, hsup2/C32A38am, hsup2/C32A38oc or hsup2/C32A38op tRNA and, when present, E. coli GlnRS (EcQRS) or E. coli TrpRS (EcWRS). The RLucFLuc fusion protein was detected with an anti-RLuc antibody. *, protein cross-reacting nonspecifically with anti-RLuc antibody.

### DEFINITIONS

The following definitions are provided to facilitate understanding of the invention. Unless otherwise defined, all scientific and technical terms are understood to have the same meaning as commonly used in the art to which they pertain. As used herein, the singular forms “a”, “an” and “the” include plural referents unless the context clearly indictates otherwise. Thus, for example, reference to “a molecule” optionally includes a combination of two or more such molecules, and the like. The terms “tRNA synthetase” and “aminoacyl tRNA synthetase” (aaRS) are used interchangeably herein.

Approximately: As used herein, the terms approximately or about in reference to a number are generally taken to include numbers that fall within a range of 5% in either direction (greater than or less than) of the number unless otherwise stated or otherwise evident from the context (except where such number would exceed 100% of a possible value).

Disease state: For the purposes of the present invention, a “disease state” or “disease phenotype” is a characteristic of a mammalian cell that results from a stop codon within the coding region of a gene inside the cell (e.g., that results from a nonsense mutation). For example, an increasing number of human genetic diseases are thought to be caused by nonsense mutations (see, for example, Atkinson et al., Nuc. Acids Res. 22:1327, 1994). To give but a few examples, β-thalessemia, Duchenne muscular dystrophy, xeroderma pigmentosum, Fanconi's anemia, and cystic fibrosis can all be caused by nonsense mutations in identified genes.

Endogenous tRNA synthetase: A tRNA synthetase is considered to be “endogenous” to a cell if it is present in the cell into which a tRNA is introduced according to the present invention. As will be apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

Heterologous tRNA synthetase: A tRNA synthetase is considered to be “heterologous” to a cell if it is not naturally found in cells of the relevant type, i.e., if the particular cell (or an ancestor of the cell) has been engineered or otherwise manipulated by the hand of man to contain or express it.

Native tRNA synthetase. A tRNA synthetase is considered to be “native” to a cell if it is naturally found in cells of the relevant type. Unless otherwise indicated, a “native mammalian aminoacyl-tRNA synthetase” refers to an aminoacyl-tRNA synthetase that is naturally found in the cytoplasm of a mammalian cell.

Endogenous tRNA: A tRNA is considered to be “endogenous” to a cell if it is present in the cell into which a tRNA is introduced according to the present invention. As will be apparent to those of ordinary skill in the art, a tRNA may be considered to be endogenous for these purposes whether it is naturally found in cells of the relevant type, or whether the particular cell at issue has been engineered or otherwise manipulated by the hand of man to contain or express it.

Heterologous tRNA: A tRNA is considered to be “heterologous” to a cell if it is not naturally found in cells of the relevant type, i.e., the particular cell (or an ancestor of the cell) has been engineered or otherwise manipulated by the hand of man to contain or express it.

Native tRNA: A tRNA is considered to be “native” to a cell if it is naturally found in cells of the relevant type. Unless otherwise indicated, a “native mammalian tRNA” refers to a tRNA that is naturally found in the cytoplasm of a mammalian cell.

Heterologous polynucleotide: A polynucleotide is considered to be heterologous to a cell if it is not naturally found in cells of the relevant type, i.e., the particular cell (or an ancestor of the cell) has been engineered or otherwise manipulated by the hand of man to contain or express the polynucleotide. The polynucleotide may, but need not be, identical in sequence to at least a portion of a polynucleotide that is naturally found in the cell. The polynucleotide may encode a polypeptide that is naturally found in the cell or a polypeptide that is not naturally found in the cell (a heterologous polypeptide). A polynucleotide that is introduced into a cell (or an ancestor of the cell) and comprises an open reading frame containing a stop codon in place of a codon that would be found in a naturally occurring counterpart of the polynucleotide is an example of a heterologous polynucleotide. A suppressor tRNA of the invention that is introduced into a cell is also a heterologous polynucleotide.

Suppressor tRNA: A “suppressor tRNA” is one whose anti-codon is complementary with a codon that would otherwise terminate translation, so that detectable read-through occurs under the conditions of interest. Standard termination codons are amber (UAG), ochre (UAA), and opal (UGA) codons. However, non-standard termination codons (e.g., 4-nucleotide codons) have also been employed in the literature (see, for example, Moore et al., J. Mol. Biol. 298:195, 2000; Hohsaka et al., J. Am. Chem. Soc. 121:12194, 1999) and are of use in certain embodiments of the invention. Termination codons are also referred to as stop codons or nonsense codons.

Unnatural amino acid: An “unnatural amino acid” is any amino acid other than the 20 naturally-occurring amino acids found in naturally occurring proteins, and includes amino acid analogues. In general, any compound that can be incorporated into a polypeptide chain can be an unnatural amino acid. Preferably, such compounds have the chemical structure H₂N—CHR—CO₂H. The alpha-carbon may be in the L-configuration, as in naturally occurring amino acids, or may be in the D-configuration.

Gene: For the purposes of the present invention, the term “gene” has its meaning as understood in the art, i.e., a polynucleotide (typically DNA) that encodes a particular a polypeptide or a structural or funtional RNA molecule such as a tRNA In general, a gene is taken to include gene regulatory sequences (e.g., promoters, enhancers, etc.) and/or intron sequences, in addition to coding sequences (open reading frames). A “gene product” or “expression product” is, in general, an RNA transcribed from the gene (e.g., either pre- or post-processing) or a polypeptide encoded by an RNA transcribed from the gene (e.g., either pre- or post-modification). A gene or polynucleotide is said to “encode” an RNA or polypeptide expression product. The present invention refers to genes having one or more stop codons in the open reading frame. It is to be understood that in this context the open reading frame is still referred to as an open reading frame, notwithstanding that it contains a stop codon. In general, such a gene differs from a naturally occurring or “wild type” counterpart in that it contains a stop codon in what would otherwise be a naturally occurring or “wild type” open reading frame. Thus, portions of a functional protein are typically encoded both upstream and downstream of the stop codon(s).

Isolated: The term “isolated” means 1) separated from at least some of the components with which it is usually associated in nature; and/or 2) prepared or purified by a process that involves the hand of man; and/or 3) not occurring in nature. Any of the components of the invention may be provided in isolated and/or purified form. The use of the term “isolated” with respect to a mammalian cell is intended to disclaim any intent to patent a human being.

Linked: The term “linked”, or “attached” when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent.

Operably linked: The term “operably linked” or “operably associated” refers to a relationship between two nucleic acid sequences wherein the expression of one of the nucleic acid sequences is controlled by, regulated by, modulated by, etc., the other nucleic acid sequence, or a relationship between two polypeptides wherein the expression of one of the polypeptides is controlled by, regulated by, modulated by, etc., the other polypeptide. For example, the transcription of a nucleic acid sequence is directed by an operably linked transcriptional regulatory sequence such as a promoter sequence; post-transcriptional processing of a nucleic acid is directed by an operably linked processing sequence; the translation of a nucleic acid sequence is directed by an operably linked translational regulatory sequence; the transport, stability, or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence, or a polypeptide that is operably linked to a second polypeptide, is covalently linked, either directly or indirectly, to such a sequence, although any effective three-dimensional association is acceptable.

Orthogonal: The term “orthogonal” refers to a tRNA or an aminoacyl-tRNA synthetase that is used with or operates with reduced efficiency by or in a system of interest (e.g., an in vitro translation system, a cell, etc.) unless the system has been supplemented with or manipulated to contain or express an aaRS capable of aminoacylating the tRNA, or a tRNA that can serve as a substrate for the aminoacyl tRNA synthetase, respectively. Orthogonal refers to the inability or reduced efficiency of an orthogonal tRNA or orthogonal aminoacyl-tRNA synthetase to function in the translation system of interest in unless the system has been supplemented with or manipulated to contain or express an appropriate aaRS or tRNA, respectively. For example, an orthogonal aminoacyl tRNA synthetase in a translation system of interest aminoacylates an endogenous tRNA in the translation system of interest with reduced or even zero efficiency, when compared to aminoacylation of such an endogenous tRNA by an endogenous aminoacyl tRNA synthetase. An orthogonal tRNA in a translation system of interest is aminoacylated by an endogenous aminoacyl-tRNA synthetase in the translation system of interest with reduced or even zero efficiency, when compared to aminoacylation of an endogenous tRNA by an endogenous aminoacyl-tRNA synthetase.

It is to be understood that when an orthogonal tRNA is introduced into or expressed in a translation system, it will typically be the case that an aaRS capable of aminoacylating the tRNA will also be introduced into or expressed in the translation system such as a cell prior to or following introduction or expression of the orthogonal tRNA. Such an aaRS is, of course, not considered endogenous to the system in this context. Similarly, it is to be understood that when an orthogonal aaRS is introduced into or expressed in a translation system such as a cell, it will typically be the case that one or more tRNAs that can be aminoacylated by the aaRS will be introduced into or expressed in the system. Such tRNAs are, of course, not considered endogenous to the system in this context. An orthogonal tRNA or orthogonal aaRS in a system of interest may be referred to as being orthogonal “in” the system of interest, or orthogonal “to” the system of interest.

A useful way to determine whether a suppressor tRNA is orthogonal to a system of interest is to introduce the tRNA into the system either in non-aminoacylated form or in aminoacylated form and to measure the relative ability of the tRNA to suppress the relevant stop codon. If the tRNA is orthogonal, then suppression by the non-aminoacylated tRNA typically occurs at a level of 20% or less, 10% or less, 5% or less, preferably approximately 1-2% or less, e.g., less than 1%, of the level of suppression achieved by the aminoacylated tRNA. As will be recognized by one of ordinary skill in the art, if suppression occurs at an insignificant level in a system of interest, the tRNA is considered not to be a substrate for any aaRS in the system. Generally, suppression by the non-aminoacylated orthogonal tRNA is close to the background level of suppression (i.e., the level of suppression measured in the absence of the suppressor tRNA), as compared with the level of suppression that would be achieved by an aminoacylated tRNA or the level of amino acid incorporation that would be achieved by a tRNA for which a cognate aaRS is present in the system. A variety of methods for determining whether a tRNA or aaRS is orthogonal to a system of interest are known to one of ordinary skill in the art (Kowal, A., et al., Proc. Natl. Acad. Sci. USA, 98, 2268-2273, 2001; Varshney, U., et al., J. Biol. Chem., 266(36) 24712-24718, 1991; Drabkin, H. J., et al., Mol. Cell. Biol. 16, 907-913, 1996.). In general, if an aaRS that is introduced into or expressed in a translation system aminoacylates an endogenous tRNA with an efficiency that is 5% or less, preferably approximately 1-2% or less, e.g., less than 1% of the efficiency with which the endogenous tRNA is aminoacylated by an endogenous aaRS, i.e., the aaRS would be considered by one of ordinary skill in the art to be orthogonal to the system. As will be recognized by one of ordinary skill in the art, if suppression by an endogenous tRNA occurs at an insignificant level in a system of interest, the introduced or expressed aaRS is considered not to utilize any endogenous tRNA as a substrate. Typically, aminoacylation of an endogenous tRNA by the aaRS will be close to background levels.

For purposes of the present invention, unless otherwise indicated or otherwise evident from the context, a tRNA that is not aminoacylated by any native mammalian aminoacyl tRNA synthetase, or is aminoacylated with significantly reduced efficiency by one or more native mammalian aminoacyl tRNA synthetases relative to the efficiency with which such tRNA synthetase aminoacylates a native tRNA is considered orthogonal to a mammalian cell. Similarly, unless otherwise indicated or otherwise evident from the context, an aminoacyl tRNA synthetase that does not aminoacylate any native mammalian tRNA or aminoacylates such a tRNA with significantly reduced efficiency relative to the efficiency with which such a tRNA is aminoacylated by a native mammalian aminoacyl tRNA synthetase is considered orthogonal to a mammalian cell.

Polynucleotide: The term “polynucleotide” refers to a polymer of nucleotides (typically at least 3) and is used interchangeably with “nucleic acid”. Naturally occurring nucleic acids include DNA and RNA. A nucleotide comprises a nitrogenous base, a sugar molecule, and a phosphate group. A nucleoside comprises a nitrogenous base linked to a sugar molecule. In a polynucleotide or oligonucleotide, phosphate groups covalently link adjacent nucleosides to form a polymer. The polymer may include natural nucleosides (e.g., adenosine, thymidine, guanosine, cytidine, uridine, deoxyadenosine, deoxythymidine, deoxyguanosine, and deoxycytidine), other nucleosides or nucleoside analogs, nucleosides containing chemically modified bases and/or biologically modified bases (e.g., methylated bases), intercalated bases, modified sugars, etc. The phosphate groups in a polynucleotide or oligonucleotide are typically considered to form the internucleoside backbone of the polymer. In naturally occurring nucleic acids (DNA or RNA), the backbone linkage is via a phosphodiester bond. However, polynucleotides and oligonucleotides containing modified backbones or non-naturally occurring internucleoside linkages can also be used in the present invention. Such modified backbones include ones that have a phosphorus atom in the backbone and others that do not have a phosphorus atom in the backbone. Examples of modified linkages include, but are not limited to, phosphorothioate and 5′-N-phosphoramidite linkages. See Kornberg and Baker, DNA Replication, 2nd Ed. (Freeman, San Francisco, 1992), Scheit, Nucleotide Analogs (John Wiley, New York, 1980),U.S. Patent Application No.20040092470 and references therein for further discussion of various nucleotides, nucleosides, and backbone structures that can be used and methods for producing them.

A polynucleotide may be of any size or sequence and may be single- or double-stranded. If single-stranded, it may be a coding or noncoding strand. Polynucleotides in the form of DNA, cDNA, genomic DNA, RNA, mRNA and synthetic DNA are or RNA are within the scope of the present invention. A polynucleotide may be, for example, a modified or unmodified circular plasmid, a linearized plasmid, a cosmid, a viral genome, a modified viral genome, an artificial chromosome, etc., or a portion of the foregoing. The polynucleotide may be isolated and/or purified and may be substantially pure. For example, the polynucleotide may be greater than 50% pure, more preferably greater than 75% pure, and most preferably greater than 95% pure. The polynucleotide may be provided by any means known in the art. In certain preferred embodiments, the polynucleotide has been derived using recombinant techniques (for a detailed description of these techniques, please see Ausubel et al., supra, or Molecular Cloning: A Laboratory Manual, supra.) The polynucleotide may also be obtained from natural sources and purified from contaminating components found normally in nature. The polynucleotide may be synthesized using enzymatic techniques, either within cells or in vitro. The polynucleotide may also be chemically synthesized. In certain embodiments, the polynucleotide is synthesized using standard solid phase chemistry. The polynucleotide may be modified by chemical or biological means. Such modifications may lead to increased stability of the polynucleotide. Modifications include methylation, phosphorylation, end-capping, etc.

The term “polynucleotide sequence” or “nucleic acid sequence” as used herein can refer to the nucleic acid material itself and is not restricted to the sequence information (i.e. the succession of letters chosen among the five base letters A, G, C, T, or U) that biochemically characterizes a specific nucleic acid, e.g., a DNA or RNA molecule. A nucleic acid sequence is presented in the 5′ to 3′ direction unless otherwise indicated. “Polynucleotide” may refer to an individual polynucleotide or a plurality of polynucleotides having a given sequence.

Polypeptide: “Polypeptide”, as used herein, refers to a polymer of amino acids. A protein is a molecule composed of one or more polypeptides. The terms “protein”, “polypeptide”, and “peptide” may be used interchangeably. The amino acids may be naturally occurring or may be unnatural amino acids. The term “polypeptide sequence” or “amino acid sequence” as used herein can refer to the polypeptide material itself and is not restricted to the sequence information (i.e. the succession of letters or three letter codes chosen among the letters and codes used as abbreviations for amino acid names) that biochemically characterizes a polypeptide. A polypeptide sequence presented herein is presented in an N-terminal to C-terminal direction unless otherwise indicated. “Polypeptide” may refer to an individual polypeptide or a plurality of polypeptides having a given sequence.

Purified: “Purified”, as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

Regulatory element: The term “regulatory element” or “regulatory sequence” in reference to a nucleic acid is generally used herein to describe a portion of nucleic acid that directs or increases one or more steps in the expression (particularly transcription, but in some cases other events such as splicing or other processing) of nucleic acid sequence(s) with which it is operatively linked. The term includes promoters and can also refer to enhancers and other expression signals such as other transcriptional control elements. Promoters are regions of nucleic acid that include a site to which RNA polymerase binds before initiating transcription and that are typically necessary for even basal levels of transcription to occur. Generally such elements comprise a TATA box. Enhancers are regions of nucleic acid that encompass binding sites for protein(s) that elevate transcriptional activity of a nearby or distantly located promoter, typically above some basal level of expression that would exist in the absence of the enhancer. In some embodiments of the invention, regulatory sequences may direct constitutive expression of a nucleotide sequence (e.g., expression in most or all cell types under typical physiological conditions in culture or in an organism); in other embodiments, regulatory sequences may direct cell or tissue-specific and/or inducible expression. For example, expression may be induced or by the presence or addition of an inducing agent such as a hormone or other small molecule, a metal, by an increase in temperature, etc. Regulatory elements may also prevent, inhibit, or decrease expression of an operatively linked nucleic acid, and their activity may be controlled by repressors, e.g., hormones, small molecules, etc.

In general, the level of expression may be determined using standard techniques for measuring mRNA or protein. Such methods include Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein array analysis, mass spectrometry, etc. A convenient way to determine expression level is to place a nucleic acid (which may be referred to as a “reporter gene”) that encodes a readily detectable marker (e.g., a fluorescent or luminescent protein such as green fluorescent protein or luciferase, an enzyme such as alkaline phosphatase, etc.), in operable association with the regulatory element in an expression vector (which is often referred to as a reporter), introduce the vector into a cell type of interest or into an organism, maintain the cell or organism for a period of time, and then measure expression of the marker, taking advantage of whatever property renders it readily detectable (e.g., fluorescence, luminescence, enzymatic activity, alteration of optical property of a substrate, etc.). Comparing expression in the absence and presence of the regulatory element indicates the degree to which the regulatory element affects expression of an operatively linked sequence.

Small molecule: “Small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Certain small molecules are useful as inducers to induce expression regulated by an inducible promoter.

Subject: “Subject”, as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

Translation System: The term “translation system” refers to the components necessary to incorporate an amino acid, e.g., a naturally occuring amino acid, into a growing polypeptide chain (protein). For example, components can include ribosomes, tRNAs, aminoacyl tRNA synthetases, amino acids, template(s) such as RNA (e.g., capped or uncapped mRNA), energy sources (e.g., ATP, GTP), energy regenerating systems (e.g., creatine phosphate and creatine phosphokinase for eukaryotic systems; phosphoenol pyruvate and pyruvate kinase for a prokaryotic lysate), and other co-factors (Mg²⁺, K⁺, etc.), buffers, etc. Similar or identical components will typically be required for the incorporation of unnatural amino acids. In vitro translation systems are known in the art and are commercially available, e.g., cell-free systems such as reticulocyte lysate translation systems, wheat germ extract translation systems, E. coli extract translation systems. Individual components of a translation system may be combined to form a complete system and/or components of a translation system may be isolated or partially purified from natural sources. In general, aaRSs and tRNAs present in an in vitro translation system prior to the addition of one or more aaRSs or tRNAs not found in the standard art-recognized in vitro translation systems are considered endogenous to such systems. In vivo (i.e., within cells) translation systems can also be used and comprise, in general, cells containing components analogous to those recited above. The components of the present invention, e.g., suppressor tRNAs and/or aminoacyl-tRNA synthetases can be added to an in vitro translation system, introduced into an in vivo translation system such as a mammalian cell, or expressed in an in vivo translation system such as a mammalian cell.

Vector: “Vector” is used herein to refer to a nucleic acid or a virus or portion thereof (e.g., a viral capsid) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part of all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof (e.g., viral capsids) that can be used to introduce nucleic acid molecules into cells are referred to as viral vectors. Useful viral vectors include adenoviruses, retroviruses, lentiviruses, vaccinia virus and other poxviruses, herpex simplex virus, and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Where sufficient information is lacking it may, but need not be, supplied by a host cell or by another vector introduced into the cell.

Expression vectors are vectors that include regulatory sequence(s), e.g., expression control sequences such as a promoter, sufficient to direct transcription of an operably linked nucleic acid. An expression vector comprises sufficient cis-acting elements for expression; other elements for expression can be supplied by the host cell or in vitro expression system. Such vectors typically include one or more appropriately positioned sites for restriction enzymes, to facilitate introduction of the nucleic acid to be expressed into the vector.

### DETAILED DESCRIPTION OF CERTAIN EMBODIMENTS OF THE INVENTION

Overview

The present invention provides novel suppressor tRNAs and methods of use thereof. In one aspect the invention provides an ochre supppressor tRNA that is orthogonal to a mammalian cell. The invention also provides an amber suppressor tRNA that is orthogonal to a mammalian cell, wherein the amber suppressor tRNA has a translation efficiency of at least 2.8%, e.g., between approximately 2.8% and approximately 34% when present in a mammalian cell that contains an aminoacyl tRNA synthetase that aminoacylates the amber suppressor tRNA. The invention also provides an opal suppressor tRNA that is orthogonal to a mammalian cell, wherein the opal suppressor tRNA has a translation efficiency of at least 0.05%, e.g., between approximately 0.05% and approximately 10% when present in a mammalian cell that contains an aminoacyl tRNA synthetase that aminoacylates the opal suppressor tRNA.

In certain embodiments of the invention the ochre suppressor has a translation efficiency of at least approximately 0.03% when present in a mammalian cell that contains an aminoacyl-tRNA synthetase that aminoacylates the ochre suppressor tRNA. For example, the ochre suppressor tRNA may have a translation efficiency of between approximately 0.03% and approximately 4.5%. In one embodiment the translation efficiency is approximately 4.5%.

The invention provides collections comprising one or more of the suppressor tRNAs, wherein the tRNAs of the collection have a range of different translation efficiencies when present in a mammalian translation system such as a mammalian cell. The collections can contain any subset of the inventive suppressor tRNAs.

The invention also provides translation systems, e.g., mammalian cells or in vitro translation systems, containing one or more of the tRNAs. Optionally the translation system also contains an aminoacyl tRNA synthetase capable of utilizing the tRNA as a substrate and incorporating the amino acid attached to the tRNA into a nascent polypeptide chain at a position defined by the presence of a stop codon that is recognized by the aminoacylated tRNA within an mRNA that encodes the polypeptide. In certain embodiments of the invention the suppressor tRNA and the aminoacyl tRNA synthetase that utilizes it as a substrate are orthogonal to a mammalian cell. In certain embodiments of the invention the stop codon is an ochre codon. In other embodiments of the invention the stop codon is an amber codon. In other embodiments of the invention the stop codon is an opal codon.

The suppressor tRNA may be derived from a bacterial tRNA. In one embodiment the tRNA is derived from a bacterial tRNA^{Gln} (i.e., a bacterial tRNA that normally utilizes glutamine as a substrate and inserts glutamine at a position defined by the presence of a codon that encodes glutamine within an mRNA that encodes the polypeptide).

The aminoacyl tRNA synthetase (aaRS) may have a sequence identical to that of a bacterial aaRS. For example, in one embodiment the aaRS is a bacterial glutaminyl-tRNA synthetase (GlnRS, QRS). In another embodiment the aaRS is a bacterial tryptophanyl-tRNA synthetase (TrpRS, WRS).

The invention provides suppressor tRNAs that are efficiently utilized as substrates by at least two different aaRSs, i.e., they are efficiently aminoacylated by at least two different aaRSs. Optionally one or more, e.g., both, of the aaRSs is/are orthogonal to a mammalian cell. For example, the invention provides a suppressor tRNA that is orthogonal to a mammalian cell and is efficiently recognized by a bacterial GlnRS and a bacterial TrpRS. In one embodiment the suppressor tRNA is an amber suppressor. In another embodiment the suppressor tRNA is an opal suppressor.

In certain embodiments of the present invention a bacterial suppressor tRNA or bacterial aaRS described herein is an E. coli tRNA or aaRS or is derived from an E. coli tRNA or aaRS.

The invention provides cells, e.g., mammalian cells, that contain or express one or more of the inventive suppressor tRNAs and/or one or more of the aaRSs that aminoacylate the inventive tRNAs. The cell may contain or express any combination of suppressor tRNAs and/or aaRSs. In certain embodiments of the invention the cell contains or expresses one or more orthogonal suppressor tRNA-aaRS pairs, e.g., 1, 2, or 3 pairs. In various embodiments of the invention the suppressor tRNAs are aminoacylated by the same aaRS, while in other embodiments of the invention they are aminoacylated by different aaRSs. The cells comprise a template for transcription of the suppressor tRNA(s) and/or aaRSs, i.e., the cells comprise a polynucleotide that encodes the suppressor tRNA(s) and/or aaRS(s). Typically the polynucleotide is a portion of a larger polynucleotide, wherein the portion that encodes a suppressor tRNA or aaRS is operably linked to expression control signals such as a promoter. The invention includes cells, e.g., mammalian cells, that comprise templates for transcription of each possible combination of any the suppressor tRNAs and/or aaRSs described herein. For example, a cell may comprise a template for transcription of an ochre suppressor tRNA, an amber suppressor tRNA, an opal suppressor tRNA, a first aaRS that is capable of aminoacylating one or more of the suppressor tRNAs, and a second aaRS that is capable of aminoacylating one or more of the suppressor tRNAs, wherein the first and second aaRSs are different. The first and second aaRSs may be capable of aminoacylating the same set of suppressor tRNAs or a different set of suppressor tRNAs. A cell may comprise any subset of the foregoing templates and may comprise more than one template of each kind.

The cell may further comprise one or more heterologous or non-heterologous polynucleotides comprising an open reading frame that encodes a polypeptide of interest, wherein the open reading frame contains one or more stop codons. Any number or kind (i.e., ochre, amber, opal) of stop codon, in any combination, can be present in the polynucleotide. For example, there may be 1, 2, 3, or more of any one or more of these stop codons in the open reading frame. The polynucleotide can be, e.g., a gene containing a promoter operably linked to the open reading frame. The promoter can be inducible or repressible. The polypeptide of interest can be any polypeptide. Exemplary polypeptides of interest are discussed below. In certain embodiments of the invention the polypeptide is one into which it is desired to incorporate unnatural amino acid(s) at one or more positions.

The invention further provides methods for incorporating an unnatural amino acid into a polypeptide of interest synthesized by a mammalian cell. The suppressor tRNA may be imported into the cell or synthesized by the cell. The cell expresses or contains an mRNA that contains a stop codon that is recognized by the suppressor tRNA. The suppressor tRNA is charged with an unnatural amino acid either prior to import into the cell or within the cell. In the former case the tRNA is imported into the cell, and the cell need not contain an aaRS capable of aminoacylating the tRNA. In the latter case the tRNA may either be imported into the cell or synthesized by the cell. If the tRNA is not charged with an unnatural amino acid prior to import into the cell, or is synthesized by the cell, the cell should contain an aaRS, e.g., a native or non-native aaRS, that is capable of aminoacylating the tRNA. In one embodiment the cell is a mammalian cell that contains an orthogonal aaRS capable of aminoacylating the tRNA. The cell may be engineered to express the tRNA, the aaRS, or both, or may be descended from such an engineered cell.

The invention provides a method for synthesizing a protein in a mammalian cell by translation of genes containing at least one stop codon within the open reading frame, the method comprising steps of: (a) providing an isolated mammalian cell containing: (i) at least one gene that includes at least one stop codon within the open reading frame; (ii) a suppressor tRNA that is orthogonal to the cell, wherein the suppressor tRNA is any of the novel suppressor tRNAs described herein; and (iii) an aminoacyl-tRNA synthetase that aminoacylates the suppressor tRNA; and (b) maintaining the cell for a period of time under conditions in which protein synthesis can occur. The suppressor tRNA is charged with an amino acid by the aaRS, and the amino acid is inserted into the protein at a position defined by the stop codon within the open reading frame. In certain embodiments of the invention the amino acid is an unnatural amino acid. The sequences of specific suppressor tRNAs of the present invention are provided in the Examples and Figures. Each such sequence, and any collection of sequences containing one or more of these sequences, is an aspect of the present invention. The phrase “conditions suitable for protein synthesis” as used herein is not intended to be limiting. The conditions may be standard culture conditions or any variations thereof compatible with protein synthesis and may include the presence of particular agents that induce or derepress synthesis of a suppressor tRNA or aaRS by the cell.

In one embodiment, the invention provides a method for synthesizing a protein in a mammalian cell by translation of genes containing at least one stop codon within the open reading frame, the method comprising steps of: (a) providing an isolated mammalian cell containing: (i) at least one gene that includes at least one ochre codon within the open reading frame; (ii) an ochre suppressor tRNA that is orthogonal to the cell; and (iii) an aminoacyl-tRNA synthetase that aminoacylates the ochre suppressor tRNA; and (b) maintaining the cell for a period of time under conditions in which protein synthesis can occur.

In another embodiment the invention provides a method for synthesizing a protein in a mammalian cell by translation of genes containing at least three different stop codons within the open reading frame, the method comprising steps of: (a) providing an isolated mammalian cell containing: (i) at least one gene that includes three different stop codons within the open reading frame; (ii) three suppressor tRNAs, wherein the suppressor tRNAs read through three different stop codons; (iii) a set of one or more aminoacyl-tRNA synthetases, wherein aminoacyl-tRNA synthetases in the set of aminoacyl-tRNA synthetases aminoacylate the suppressor tRNAs; and (b) maintaining the cell for a period of time under conditions in which protein synthesis can occur.

The methods for synthesizing a protein in a mammalian cell can include a step of contacting the cell with one or more unnatural amino acids, such that the cell takes up the unnatural amino acid and incorporates it into proteins. In some embodiments of the invention the amino acid is an analog of a naturally occurring amino acid. If desired, the cell can be cultured under conditions in which the culture medium lacks that particular amino acid, which may enhance uptake and/or utilization of the unnatural amino acid.

The invention further provides methods of synthesizing a protein in an in vitro translation system. The methods are similar to the methods described above, except that step (a) comprises providing an in vitro translation system, and step (b) comprises maintaining the system for a period of time under conditions in which protein synthesis can occur. The conditions can be any conditions under which the translation system synthesizes proteins, such conditions being known in the art.

The invention further provides proteins synthesized according to any of the inventive methods. In certain embodiments the protein contains one or more unnatural amino acids, e.g., 1, 2, 3, 4, 5, 6, or more unnatural amino acids. In one embodiment the protein contains an unnatural amino acid inserted at each of an ochre, opal, and amber stop codon within an open reading frame that encodes the protein.

The invention further provides cells that contain one or more of the inventive proteins. In certain embodiments of the invention the proteins are synthesized in the cell. In one embodiment the cell contains two different proteins, each of which comprises a different unnatural amino acid. The first protein comprises a first unnatural amino acid, wherein the first unnatural amino acid is inserted at a first type of stop codon (e.g., an ochre codon), and the second protein comprises a second unnatural amino acid, wherein the second unnatural amino acid is inserted at a second type of stop codon (e.g., an amber or opal codon). In another embodiment the cell contains three different proteins, each of which comprises a different unnatural amino acid. The first protein comprises a first unnatural amino acid, wherein the first unnatural amino acid is inserted at a first type of stop codon (e.g., an ochre codon). The second protein comprises a second unnatural amino acid, wherein the second unnatural amino acid is inserted at a second type of stop codon (e.g., an amber codon). The third protein comprises a third unnatural amino acid, wherein the third amino acid is inserted at a third type of stop codon (e.g., an opal codon).

The invention further provides methods for identifying orthogonal suppressor tRNAs. In one embodiment the method comprises providing a tRNA having an anticodon whose sequence is altered so that it is complementary to a stop codon, e.g., an ochre codon. The tRNA can be, e.g., a bacterial tRNA. The method further comprises (i) altering one or more nucleotides in the sequence of the tRNA; (ii) testing the tRNA to determine whether it is aminoacylated by any mammalian aaRS; and (iii) selecting the tRNA as an orthogonal suppressor tRNA if the tRNA is not significantly aminoacylated by any mammalian aaRS. The method may further comprise (iv) testing the tRNA to determine whether it is aminoacylated by any non-mammalian aaRS; and (v) selecting the tRNA and the non-mammalian aaRS as being orthogonal to a mammalian cell if the tRNA is aminoacylated by the non-mammalian aaRS and the non-mammalian aaRS does not significantly aminoacylate any mammalian tRNA. Suitable methods for testing suppressor tRNAs and aaRSs are described in the Examples.

Import of Transfer RNAs (tRNAs)

In some embodiments of the invention one or more tRNAs is transported into a mammalian cell. The teachings of the present invention with respect to transport of transfer RNA into mammalian cells (also referred to herein as “import” of tRNAs into mammalian cells) are applicable to any tRNA that can be synthesized outside a mammalian cell and subsequently introduced into the cell. As noted herein, certain preferred tRNAs recognize standard nonsense codons. Some preferred tRNAs are aminoacylated prior to import, optionally with an unnatural amino acid. Also, in certain preferred embodiments of the invention, the tRNA employed is not a substrate for any tRNA synthetases present within the cell into which the tRNA is introduced. Thus in certain preferred embodiments of the invention the tRNA may not be a substrate for any tRNA synthetase present in the cell in the cellular compartment into which the tRNA is introduced, e.g., any cytoplasmic tRNA. In such embodiments, when an aminoacylated tRNA is delivered to a cell and contributes its amino acid to a growing polypeptide chain, it cannot be re-aminoacylated within the cell. For example, the present invention demonstrates that the E. coli supF tRNA is not a substrate for mammalian tRNA synthetases. In other embodiments of the invention, the tRNA is a substrate for a tRNA synthetase within the cell into which the tRNA is introduced.

Where tRNAs aminoacylated prior to introduction into the cell are utilized, the aminoacyl linkage should preferably be stable under the conditions of transport.

Amino Acids

As mentioned above, in certain embodiments of the invention, tRNAs are aminoacylated prior to being introduced into mammalian cells or into a translation system. Any amino acid or amino acid analog may be utilized to aminoacylate tRNAs in accordance with the present invention. In certain preferred embodiments of the invention, unnatural amino acids are used. For instance, it may be desirable to introduce an unnatural amino acid containing a detectable moiety (e.g., fluorophore, chromophore, or radioactive group), a photoactivatable group, or a heavy atom (e.g., iodine). Alternatively or additionally, amino acids including chemically reactive moieties could be used.

For example, a naturally occurring amino acid (e.g., glutamine, tyrosine, tryptophan, etc.) may be modified, e.g., by the attachment or incorporation of a chemical entity such as a carbohydrate group, a phosphate group, a farnesyl group, an isofarnesyl group, a fatty acid group, a linker for conjugation, functionalization, reactive group, fluorophore, or other modification, etc.

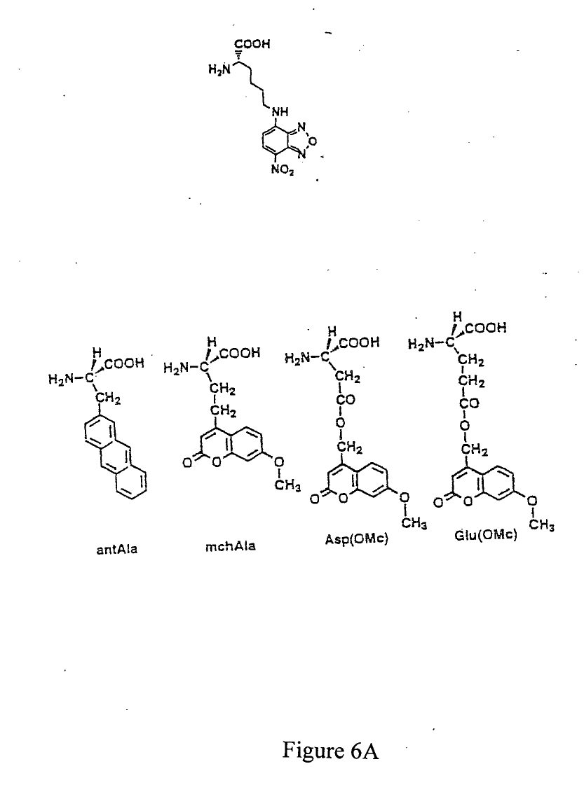

FIG. 6 presents exemplary structures of certain unnatural amino acids that could be used in accordance with the present invention; those of ordinary skill on the art will readily appreciate that any of a variety of other compounds could also be used. See, e.g., See, e.g., Barrett, G. (ed.) Amino Acid Derivatives: A Practical Approach (Practical Approach Series), Oxford University Press (1999), U.S. Publication Nos. 20030082575, 20030108885, and W02004026328 for numerous nonlimiting examples.

tRNAs may also be aminoacylated after introduction into a translation system (e.g., an in vitro translation system, a cell, etc.). The amino acid, either natural or unnatural, and an aaRS capable of aminoacylating the tRNA, must also be present. If the translation system does not already contain such an aaRS, it can be directly introduced into the system of interest or expressed in the system as described below.

Introducing tRNA into Cells

Any available method may be used in accordance with the present invention to introduce synthesized tRNAs into mammalian cells. In preferred embodiments of the invention, tRNAs are imported into cells using cellular machinery, and are not introduced into the cell lumen by mechanical means such as injection. In general, import processes are characterized by being competable and/or inhibitable. Import offers several advantages over other methods for introducing tRNAs into cells. For example, tRNAs can be imported into multiple cells simultaneously. By contrast, when injection is utilized, (e.g., into Xenopus oocytes) individual cells must be injected individually. Also, import may achieve higher levels of tRNA within cells, thereby allowing higher levels of production of protein. In particularly preferred embodiments of the invention, tRNAs are introduced into mammalian cells using Effectene or Lipofectamine in conjunction with a nucleic acid condensing enhancer. Without wishing to be bound by any particular theory, we propose that the nucleic acid condensing enhancers render nucleic acids more compact and therefore easier to import. Such an agent is not necessarily required of course, so long as the conditions used do in fact achieve import. Other methods, e.g., electroporation, microinjection, etc., can also be used to introduce tRNAs into cells (Monahan, S. L., et al., Chem. Biol. 10, 573-580, 2003; Ilegems, E., et al., Nucleic Acids Res. 30, e128, 1-6, 2002). The inventive methods may be used with any mammalian cells or cell lines, e.g., CHO, R1.1, B-W, L-M, African Green Monkey Kidney cells (e.g. COS-1, COS-7, BSC-1, BSC-40 and BMT-10), cultured human cells, etc.

Reporter System

The invention includes a reporter system that can be used to identify additional suppressor tRNAs and/or additional aaRSs that aminoacylate suppressor tRNAs and to evaluate the efficiency with which a suppressor tRNA is utilized or the efficiency with which an aaRS utilizes a suppressor tRNA. In particular, the reporter system is useful for identifying suppressor tRNA combinations and suppressor tRNA/aaRS pairs that function to suppress at least two different stop codons in a single protein with sufficiently high efficiency that the protein can be produced in non-negligible amounts.

In certain embodiments, the reporter system comprises a polynucleotide that encodes a protein having first and second domains, each of which serves as a readily detectable marker, wherein the markers are distinguishable from one another (e.g., they produce detectably different signals). The sequence that encodes the first domain lacks stop codons and is thus translated by the native tRNAs and aaRSs that exist in a mammalian cell. The second domain, which is located 3′ to the first domain, contains two or more different stop codons in the open reading frame. Thus production of a full length protein requires presence of suppressor tRNAs that read through each stop codon and also requires that the suppressor tRNAs are aminoacylated, either prior to introduction into the cell or within the cell. In the latter case, the cell must express one or more aaRSs, such that each of the stop codons can be aminoacylated in the cell. The stop codons are appropriately positioned such that a truncation protein resulting from termination at each codon either is not readily detectable or, if detectable, produces a signal that differs from that produced by the full length protein, so that it is possible to determine whether readthrough of both stop codons has occurred. The first readily detectable marker serves as an internal control, e.g., for overall transcription and/or translation efficiency and allows for comparison of different suppressor tRNAs, aaRSs, etc.

In general, a readily detectable marker is a marker whose presence within a cell can be detected through means other than subjecting the cell to a selective condition or directly measuring the amount of the marker itself. Thus, the expression of a detectable marker within a cell results in the production of a signal that can be detected and/or measured. The process of detection or measurement may involve the use of additional reagents and may involve processing of the cell. For example, where the detectable marker is an enzyme, detection or measurement of the marker will typically involve providing a substrate for the enzyme. Preferably the signal is a readily detectable signal such as light, fluorescence, luminescence, bioluminescence, chemiluminescence, enzymatic reaction products, or color. Suitable markers include, for example, chloramphenicol acetyltransferase, green fluorescent protein and variants thereof. Other detectable markers that produce a fluorescent signal include red, blue, yellow, cyan, and sapphire fluorescent proteins, reef coral fluorescent protein, etc. A wide variety of such markers is available commercially, e.g., from BD Biosciences (Clontech). Additional detectable markers include luciferase derived from the firefly (Photinus pyralis) or the sea pansy (Renilla reniformis).

Orthogonal tRNA Ochre Suppressors

The invention provides a variety of novel ochre suppressor tRNAs that do not serve as a substrate when present in a mammalian cell but that function in such cells when the cells also contain a suitable non-native aaRS. The ochre suppressors were derived from E. coli tRNA^{Gln} but differ from the naturally occurring sequence in a variety of ways as described in detail in the Examples. In particular, certain of the suppressor tRNAs contain mutations that greatly increase the efficiency with which they suppress ochre codons in mammalian systems. The invention thus provides a set of ochre suppressor tRNAs having a wide range of activities. Without wishing to be bound by any theory, the availability of such a range of activities may be useful to control the level of protein producted by readthrough of stop codon(s) in a cell and/or to minimize the likelihood of toxicity that may arise either as a result of production of the protein itself or as a result of expression of the inventive suppressor tRNA(s) and/or aaRS(s).

It will be appreciated that in certain embodiments of the invention the novel ochre suppressors tRNAs can be used with a variety of different aaRSs, including aaRSs that aminoacylate the ochre suppressor tRNAs with any of a variety of different unnatural amino acids.

The invention provides a mammalian cell that contains one or more of the different ochre suppressor tRNAs and, optionally, an aaRS that aminoacylates them. The mammalian cell may express the suppressor tRNA, or the suppressor tRNA may have been synthesized outside the cell and imported into it. The invention further provides polynucleotides that comprise a template for synthesis of the suppressor tRNA. Preferably such polynucleotides comprise a promoter suitable for synthesis of a tRNA, e.g., an RNA polymerase III promoter, operably linked to the tRNA gene. The invention further provides expression vectors (e.g., DNA plasmids) comprising such polynucleotides. The mammalian cell may express the suppressor tRNA in a transient or stable, e.g., heritable, manner. In the latter case the template and operably linked promoter are typically incorporated into the genome of the cell.

Complete Sets of Orthogonal Aminoacyl tRNA Synthetase—Amber, Ochre, and Opal Suppressor Pairs

The invention further provides an orthogonal amber suppressor/aaRS pair and an orthogonal opal suppressor/aaRS pair, thus resulting in what the inventors believe to be the first set of orthogonal pairs that can suppress amber, ochre, and opal codons in a mammalian cell. The amber and opal suppressor tRNAs were derived from E. coli tRNA^{Gln} but differ from the naturally occurring sequence in a variety of ways as described in detail in the Examples. In particular, certain of the suppressor tRNAs contain mutations that greatly increase the efficiency with which they suppress amber or opal codons, respectively, in mammalian systems. The invention thus provides a set of amber suppressor tRNAs having a wide range of activities and a set of opal suppressor tRNAs having a wide range of activities. Without wishing to be bound by any theory, the availability of such a range of activities may be useful to control the level of protein producted by readthrough of stop codon(s) in a cell and/or to minimize the likelihood of toxicity that may arise either as a result of production of the protein itself or as a result of expression of the inventive suppressor tRNA(s) and/or aaRS(s). In addition, without wishing to be bound by any theory, the high suppression activity of certain of the ochre, amber, and opal suppressors is likely to be of considerable importance in terms of producing proteins containing one or more unnatural amino acids in significant quantities in mammalian cells, particularly for producing proteins containing two or three unnatural amino acids, which typically requires suppression of three different termination codons. To the best of the inventors' knowledge, the results described herein represent the first demonstration of suppression of three different termination codons in an mRNA.

The invention further provides mammalian cells, polynucleotides, and expression vectors containing, expressing, or encoding one or more of the amber suppressor tRNAs, opal suppressor tRNAs, and/or aaRS, as described above for the ochre suppressor tRNA/aaRS pair.

The ochre, amber, and opal suppressors tRNAs can be used with a variety of different aaRSs, including aaRSs that aminoacylate the suppressor tRNAs with any of a variety of different unnatural amino acids. For example, in one embodiment a glutaminyl-tRNA synthetase (GlnRS, QRS) is used to aminoacylate an ochre, amber, or opal suppressor tRNA of the present invention. In another embodiment a bacterial tryptophanyl-tRNA synthetase (TrpRS, WRS) is used. The latter may be of particular use for aminoacylating an amber or opal suppressor of the present invention.

In certain embodiments of the invention one or more of the inventive suppressor tRNA-aaRS systems, e.g., an orthogonal ochre suppressor tRNA-aaRS pair, may be used in combination with either an E. coli TyrRS-Bacillus stearothermophilus (B.st.) tRNA^{Tyr} derived amber suppressor (Sakamoto, K., et al., Nucleic Acids Res., 30, 4692-4699, 2002) and/or a B. subtilis (B.s.) TrpRS-B.s. tRNA^{Trp} derived opal suppressor system (Zhang, Z., et al., Proc. Natl. Acad. Sci. U.S.A., 101, 8882-8887, 2004). The invention provides mammalian cells containing an inventive suppressor tRNA-aaRS pair and one or more of these pairs. In certain embodiments the invention can also be used in conjunction with systems that have been developed in an effort to expand the genetic code. See, e.g., Wang, L., et al., Science, 292, 498-500, 2001, Chin, J. W., et al., Science, 301, 964-967, 2003; Anderson, J. C., et al, Proc. Natl. Acad. Sci. U.S.A., 101, 7566-7571, 2004.

Regulatable Expression

As described in the Examples, the amber, ochre and opal suppressor tRNAs of the invention, expressed in mammalian cells, are specific for their cognate codons, and their activity in suppression is essentially totally dependent upon expression of a heterologous aaRS that aminoacylates them. Thus in certain embodiments of the invention suppression of the amber, ochre and opal codons in mammalian cells is regulated by regulating the expression of the aaRS. Regulatable expression can be achieved utilizing regulatable expression signals, e.g., an inducible or repressible promoter. A wide variety of such promoter systems are known in the art. For example, tetracycline-regulated suppression can be used (Park, H. J. and RajBhandary, U. L. Mol. Cell. Biol., 18, 4418-4425, 1988; Corbel, S., and Rossi, F., Curr Opin Biotechnol., 13(5):448-52, 2002). Other systems employ promoters that are responsive to other small molecules, synthetic or naturally occurring glucocorticoids or other hormones, temperature, metals, etc. Without wishing to be bound by any theory, the ability to regulate suppression may be useful to minimize any potential toxicity arising from expression of the inventive suppressor tRNAs and/or aaRSs. Thus, in certain embodiments of the invention the suppressor tRNAs, aaRSs, or proteins carrying one or more unnatural amino acid are not produced constitutively in a mammalian cell. Instead, their production may be repressed due to the presence of a repressing agent so that their production is induced upon removal of the repressing agent. Alternately, their production may require the presence of an inducing agent or condition. It may, however, be desirable to utilize constitutive promoters or strong promoters or promoter/enhancers, e.g., a CMV promoter/enhancer or SV40 promoter in order to achieve high expression of an aaRS. Therefore, the invention provides polynucleotides and expression vectors in which a sequence coding for an aaRS is under control of any of a wide variety of regulatory elements.

Kits

The invention features kits comprising one or more of the inventive suppressor tRNAs and/or a polynucleotide or expression vector that comprises a template for synthesis of an inventive suppressor tRNA. The kits may contain one or more additional items. For example, the kits may contain: (i) one or more aaRSs that aminoacylate an inventive suppressor tRNA; (ii) a mammalian cell; (iii) an unnatural amino acid; (iv) a transfection reagent such as a lipid; (v) an in vitro translation system; (vi) a reporter system; (vii) a buffer; (viii) tissue culture medium; (ix) an agent that induces or represses transcription; (x) instructions for use of the kit. All of these items, or any subset thereof, may be present in the kit. Other components mentioned herein or not mentioned herein may also be included.

In certain embodiments the kit contains an ochre suppressor tRNA, or a polynucleotide or expression vector comprising a template for synthesis thereof, or both. The kit may further contain (i) an amber suppressor tRNA or a polynucleotide or expression vector comprising a template for synthesis thereof, or both; and/or an opal suppressor tRNA or a polynucleotide or expression vector comprising a template for synthesis thereof. The suppressor tRNAs may be orthogonal to a mammalian cell. The amber suppressor tRNA may, but need not, have a translation efficiency of between approximately 2.8% and approximately 34%. The opal suppressor tRNA may, but need not, have a translation efficiency of between approximately 0.05% and approximately 10%. In certain embodiments the invention contains a complete set of orthogonal suppressor tRNAs (ochre, amber, and opal) for use in a mammalian system, and, optionally, one or more aaRSs that aminoacylate one or more of the suppressor tRNAs.

The kit may contain a mammalian cell that expresses one or more aaRSs capable of aminoacylating an orthogonal suppressor tRNA. In certain embodiments of the invention the mammalian cell expresses two different aaRSs.

The kits of the invention may contain any one or more suppressor tRNAs, aaRSs, mammalian cells, polynucleotides, or expression vectors of this invention, in any combination. The kits may further include one or more suppressor tRNAs and/or aaRSs known in the art.

Kits may include one or more vessels or containers so that certain of the individual reagents may be separately housed. The kits may also include a means for enclosing the individual containers in relatively close confinement for commercial sale, e.g., a plastic box, in which instructions, packaging materials such as styrofoam, etc., may be enclosed.

Uses

The compositions and methods of the present invention have a number of different uses ranging from screening assays to identify and test new drug candidates to the study and manipulation of fundamental cellular processes.

INTRODUCING NON-NATURAL AMINO ACIDS INTO PROTEINS. As noted above, the inventive techniques and reagents may be used to introduce one or more unnatural amino acids into proteins. Any tRNA may be utilized, along with any unnatural amino acid. For example, in certain embodiments the unnatural amino acid is derived from glutamine. In other embodiments the unnatural amino acid is derived from tryptophan. In certain embodiments of the invention the resulting protein comprises at least one unnatural amino acid derived from glutamine and at least one unnatural amino acid derived from tryptophan. In certain embodiments of the invention the unnatural amino acid is usable as a substrate by a bacterial GlnRS or a bacterial TrpRS.

Certain embodiments of the methods for introducing unnatural amino acids into proteins utilize tRNAs that are aminoacylated prior to import into cells. Preferably, such tRNAs are not substrates for endogenous tRNA synthetases, e.g., native tRNA synthetases. Other embodiments of the methods for introducing unnatural amino acids into proteins or of suppressing stop codons utilize tRNAs that are not aminoacylated prior to import. Preferably, when tRNAs are imported into cells, such tRNAs are not substrates for native aminoacyl tRNA synthetases but are substrates for a heterologous aminoacyl tRNA synthetase present in the cell, preferably an orthogonal aminoacyl tRNA synthetase that does not significantly aminoacylate native tRNAs. Expression of the heterologous aminoacyl tRNA synthetase may be under control of a regulatable promoter, e.g., an inducible or repressible promoter. The cell may be a recombinant cell engineered to express the heterologous aaRS, as described above.

The reagents described herein can also be used to introduce unnatural amino acids into proteins in vitro, e.g., in an in vitro translation system

Proteins produced according to the methods of the present invention, e.g., proteins comprising one or more unnatural amino acids may be synthesized in vitro or within cells. In the former case, if desired, the proteins may be introduced into cells following their synthesis. A variety of methods may be used to introduce proteins into cells. For example, the protein can be microinjected into the cell. Alternately, the protein can comprise a “protein transduction domain” or a domain comprising a “cell penetrating peptide”. Such domains facilitate uptake of proteins by mammalian cells. For example, a variety of arginine-rich peptides, including peptides derived from the HIV Tat gene, are known to enhance transport across the plasma membrane. See, e.g., Langel, U. (ed.), “Cell-Penetrating Peptides: Processes and Applications”, CRC Press, Boca Raton, Fla., 2002, for further discussion. A protein comprising such a domain can be incubated with cells, which then take it up spontaneously. In many embodiments of the present invention the protein is synthesized within cells, as described above.

Any protein can be synthesized according to the methods of the present invention. Proteins of particular interest include, but are not limited to, proteins that are naturally expressed by mammalian cells and variants thereof, e.g., naturally occurring or artificially created mutants. Proteins having any of a variety of different enzymatic activities are of interest. For example, kinases (serine, threonine, and/or tyrosine kinases), phosphatases, proteases, nucleases, ATPases, GTPases, polymerases, ligases, helicases, replicases, acetylases, and transferases are of interest. Proteins involved in cell signaling processes, e.g., hormones, neurotransmitters, cytokines, chemokines, cell surface receptors, cytoplasmic or nuclear receptors, proteins having transmembrane domains, G protein coupled receptors, neurotransmitter receptors, receptors for compounds of therapeutic utility or ligands of such receptors, proteins that mediate cell-cell interactions, and proteins that mediate interactions between cells and the extracellular matrix, are also of interest. Also of interest are proteins expressed by infectious agents such as viruses.

Introduction of unnatural amino acids into proteins or polypeptides in accordance with the present invention has a wide variety of uses (Kohrer, C. and RajBhandary, U. L. Proteins carrying one or more unnatural amino acids. Chapter 33. In Ibba, M., Francklyn, C. and Cusack, S. (eds.), Aminoacyl-tRNA Synthetases, Landes Bioscience, 2004, the entirety of which is incorporated herein by reference). For example, such methods can be useful to probe the mechanical and/or functional characteristics of protein structure. For example, incorporation of detectable (e.g., fluorescent) moieties can allow the study of protein movement within and without cells. Alternatively or additionally, incorporation of reactive moieties (e.g., photoactivatable groups) can be used to identify interaction partners and/or to define three-dimensional structural motifs. Also, incorporation of amino acids such as phosphotyrosine, phosphothreonine, or phosphoserine, or analogs thereof, can be used to study cell signalling requirements.

For example, the insertion of two different analogues containing fluorescent moieties would allow the use of FRET to study protein conformation and dynamics in cells. In combination with imaging and fluorescence microscopy of cells, such fluorescence reporters can be used as biosensors. Mutants of Aequorea victoria green fluorescent protein (GFP) have been used as FRET-pairs and as biosensors of protein kinases in mammalian cells (Ting, A. Y., et al., Proc. Natl. Acad. Sci. USA 98, 15003-15008, 2001; Zhang, J., et al., Proc. Natl. Acad. Sci. USA 98, 14997-15002, 2001). The reporter proteins contained cyan fluorescent protein (CFP) at one end and yellow fluorescent protein (YFP) at the other end, with a linker consisting of an SH2 phosphotyrosine binding domain and a consensus substrate sequence -PYAQP- for the tyrosine kinase being probed. Phosphorylation of the consensus substrate led to intramolecular binding of the SH2 domain to the phosphorylated peptide segment and to a change in distance between CFP and YFP, as detected by a change in FRET. While the results obtained were striking, it is desirable to also investigate the use of small molecules as FRET-pairs in vivo, instead of large molecules such as GFPs. For example, in vitro work using cCrkII as a biosensor of Ab1 (Abelson Leukemia Virus) tyrosine kinase yielded different results depending upon the use of CFP/YFP versus fluorescein/rhodamine as FRET-pairs (Hofmann, R. M., et al, Bioorg. Med. Chem. Lett. 11, 3091-3094, 2001; Kurokawa, K., et al., J. Biol. Chem. 276, 31305-31310). Such small molecules can be attached to amino acids (either natural amino acids or amino acid analogs), thereby obtaining unnatural amino acid(s) that can then be incorporated into a protein using the reagents and methods described herein.

In accordance with the invention, introduction of two different phosphorylated amino acid analogues into a kinase, e.g., a MAP kinase may also provide a general method for activating a specific signal transduction pathway in the absence of upstream or extracellular signals. MAP kinases, which are multifunctional serine-threonine kinases, are activated by a cascade of phosphorylations leading to phosphorylation of threonine and tyrosine in the sequence -TXY- in the MAP kinase (Hunter, T., Cell 100, 113-127, 2000). Activated MAP kinases enter the nucleus where they phosphorylate and activate transcription factors. In mammals, at least twenty different MAP kinases are known (Pearson, G., et al., Endocr. Rev. 22, 153-183, 2001). The existence of such a large number of MAP kinases along with several hundreds of transcription factors in the nucleus has made it difficult to identify the relationship between an individual MAP kinase and its downstream targets (Brivanlou, A. H., and Darnell, J. E., Jr. Science 295, 813-818, 2002). Because of the central role played by phosphorylated amino acids, site-specific insertion of phospho-amino acids or phosphono-amino acids, which are more stable derivatives in vivo and excellent mimics of phospho-amino acids (Lu, W., et al., Mol. Cell 8, 759-769, 2000) represents a method for generating a constitutively activated MAP kinase without altering the protein sequence. Such constitutively activated MAP kinases could be used for a variety of analyses including comparison of gene expression profiles using DNA microarrays. Data generated from such studies would provide significant amounts of information on the patterns of downstream gene activation brought about by activation of specific MAP kinases. Such methods may also be applied to the analysis of other kinases and are of use in the identification of molecules that activate or inhibit such kinases. As is well known in the art, kinases are involved in a large number of diseases, including cancer, and there is a need in the art for improved methods of identifying agents that interact with them, e.g., activate or inhibit them.

In certain embodiments of the invention an amino acid having a nanoparticle, e.g., a metal nanoparticle or nanocluster, semiconducting nanoparticle, magnetic nanoparticle, and linked thereto is used. The nanoparticle may be responsive to an external field (e.g., an electric, electromagnetic, or magnetic field) or may be used to transduce an externally applied signal or stimulus to a polypeptide comprising the amino acid, to transmit energy to a polypeptide comprising the amino acid, to modulate the structural and/or functional characteristics of a polypeptide comprising the amino acid such as by controlling its activity, etc. See, e.g., U.S. Publication No. 20020119572 for discussion of such nanoparticles and other modulators such as chromophores and methods of use thereof

In certain preferred embodiments, the inventive system may be utilized to introduce two or more different amino acid analogues into a single protein. Such multiple modifications can be used to dissect intra-protein interactions and to study protein folding and dynamics. For example, introduction of two different fluorescent groups in the same protein allows one to use fluorescence resonance energy transfer (FRET) to analyze the three-dimensional proximity of the labelled groups in the folded protein, and whether this, proximity changes during the lifetime or activity cycle of the protein.

Alternatively or additionally, the inventive system may be utilized to read through different stop codons in different proteins within the same mammalian cell. Optionally, a different amino acid (natural or unnatural) can be introduced for each different stop codon involved.

The incorporation of two different unnatural amino acids into a protein using two different suppressor tRNAs typically involves a mRNA carrying two different termination codons within its open reading frame. This approach poses no particular problems in terms of the suppressor tRNAs also reading through the normal termination signal at the end of the reading frame. For example, the firefly luciferase gene used in the Examples contains, at the end of the reading frame, an ochre codon followed by UUC and then an amber codon. Therefore, a combination of opal and amber suppressor tRNAs can be used to incorporate two different unnatural amino acids into the protein without the suppressor tRNAs also reading through the normal termination codons, the ochre codon acting as a barrier in this case. The use of a mRNA carrying three different termination codons in the open reading frame may involve strategies for preventing readthrough of normal termination codon(s) at the end of the reading frame by the three suppressor tRNAs. The finding that in certain embodiments of the invention suppression of the ochre codon is the weakest of the three termination codons, suggests that use of a gene carrying tandem ochre termination codons at the end of the reading frame would minimize any significant readthrough of the termination codons beyond the end of the mRNA. It is noted that under the conditions used herein, there is no significant readthrough of cellular protein genes as indicated by the lack of any deleterious effects on cell viability, suggesting that the inventive methods are substantially nontoxic to mammalian cells.

CULTURE OF ANIMAL CELLS OR ANIMAL VIRUSES CARRYING NONSENSE MUTATIONS IN ONE OR MORE GENES

As described in the Examples and above, the amber, ochre and opal suppressor tRNAs of the invention, expressed in mammalian cells, are specific for their cognate codons, and their activity in suppression is essentially totally dependent upon expression of a heterologous aaRS that aminoacylates them. Cell lines carrying inducible or repressible suppressor tRNA function (e.g., inducible or repressible aaRSs and/or inducible or repressible suppressor tRNAs that are aminoacylated by such aaRSs in mammalian cells) would be particularly useful for a variety of purposes including, for example, the propagation of animal viruses carrying nonsense mutations in their genes or propagating mammalian cells with nonsense mutations in one or more genes. Such methods open up the possibility of performing genetics in mammalian cells or animal viruses, similar to manner in which the availability of bacterial nonsense suppressors has been used for genetic analysis of bacteria and bacterial viruses. Suppressor tRNAs have been used for diphtheria toxin mediated ablation of photoreceptor cells in Drosophila (Kunes, S. and Steller, H., Genes Dev., 5, 970-983, 1991) and toxin mediated ablation dependent upon suppressor tRNA function has also been suggested as a possibility for cancer therapy (Robinson, D. F. and Maxwell, I. H. (1995) Hum. Gene Ther, 6, 137-143.1995).

GENE THERAPY: Nonsense mutations are responsible for a significant number of human genetic disorders (see, for example, Atkinson et al., Nuc. Acids Res. 22:1327, 1994; Temple, G. F., et al., Nature, 296, 537-540). To give but a few examples, β-thalessemia, Duchenne muscular dystrophy, xeroderma pigmentosum, Farconi's anemia, and cystic fibrosis can all be caused by nonsense mutations in identified genes. For instance, Duchenne muscular dystrophy is caused by the absence of dystrophin protein, which may result from a nonsense mutation within the coding region of the dystrophin gene. The present invention could allow the delivery of suppressor tRNAs that, whether acylated internally or externally, would read through the stop codon and produce some level of dystrophin protein, so that disease symptoms are alleviated.

In certain preferred embodiments of the rescue of stop codon mutations in genetic diseases, tRNAs that act as substrates for endogenous tRNA synthetases are utilized; such tRNAs can be aminoacylated in vivo so that, whether or not they are aminoacylated prior to being introduced into the cells, they may be used to read through the relevant stop codon multiple times. The endogenous aminoacyl-tRNA synthetase may be a native or heterologous aaRS In the latter case, the aminoacyl tRNA synthetase or, preferably, a polynucleotide comprising a coding sequence for the aaRS operably linked to expression signals sufficient for expression in a mammalian cell, is introduced into the cell.

### EXAMPLES

### Example 1

### Import of Amber and Ochre Suppressor tRNAs into Mammalian Cells

Materials and Methods

General. Standard genetic techniques were used for cloning (Sambrook et al. Molecular Cloning: A Laboratory Manual, Cold Spring Harbor Press, Cold Spring Harbor, N.Y., Second Edition, 1989), E. coli strains DH5α (Hanahan J Mol. Biol. 166:557, 1983) and XL1-Blue (Bullock et al., BioTechniques 5:376, 1987) were used for plasmid propagation and isolation. For transfection of mammalian cells, plasmid DNAs were purified using an EndoFree Plasmid Maxi kit (Qiagen). Oligonucleotides were from Genset Oligos and radiochemicals were from New England Nuclear.

Plasmids carrying reporter genes. pRSVCAT and pRSVCATam27 and pRSVCAToc27, carrying amber and ochre mutations, respectively, at codon 27 of the chloramphenicol acetyltransferase (CAT) gene, have been described previously (Capone et al., Mol. Cell. Biol. 6:3059, 1986).

Plasmids carrying suppressor tRNA genes. The plasmid pRSVCAT/trnfM U2:A71/U35A36/G72 contains the gene for the amber suppressor derived from the E. coli tRNA^{fMet} (Lee et al.,. Proc. Natl. Acad. Sci. USA 88:11378, 1991). An ochre suppressor was generated from this plasmid by mutation of C34 to U34 in the tRNA gene using the QuikChange mutagenesis protocol (Stratagene). The plasmid pCDNA1 (Invitrogen) contains the gene for the supF amber suppressor derived from E. coli tRNA^{Tyr}₁ (Goodman et al., Nature (London) 217:1019, 1968).

Purification of suppressor tRNAs. For purification of the amber suppressor tRNA derived from E. coli tRNA^{fMet}, total tRNA (597 A₂₆₀ units) was isolated by phenol extraction of cell pellet from a 2 L culture of E. coli B105 cells (Mandal et al., J. Bacteriol. 174:7827, 1992) carrying the plasmid pRSVCAT/trnfM U2:A71/U35A36/G72 (Lee et al., Proc. Natl. Acad. Sci. USA 88:11378, 1991). The suppressor tRNA was purified by electrophoresis of 80 A₂₆₀ unit aliquots of the total tRNA on 12% non-denaturing polyacrylamide gels (0.15×20×40 cm) (Seong et al., Proc. Natl. Acad. Sci. USA 84:334, 1987). The purified tRNA was eluted from the gel with 10 mM Tris-HCl (pH 7.4) and concentrated by adsorption to a column of DEAE-cellulose followed by elution of the tRNA with 1 M NaCl and precipitation with ethanol. The same procedure was used for purification of the ochre suppressor tRNA.

supF tRNA (Goodman et al., Nature (London) 217:1019, 1968) was purified from E. Coli strain MC1061p3 carrying the plasmid pCDNA1. Total tRNA (1,000 A₂₆₀ units) isolated by phenol extraction of cell pellet from a 3 L culture was dissolved in 10 ml of buffer A [50 mM NaOAc (pH 4.5), 10 mM MgCl₂, and 1 M NaCl] and applied to a column (1.5×1.5 cm) of benzoylated and naphthoylated DEAE-cellulose (BND-cellulose) (Sigma) equilibrated with the same buffer. The column was then washed with 500 ml of the same buffer. The supF tRNA and wild type tRNA^{Tyr} were eluted with a linear gradient (total volume 500 ml) from buffer A to buffer B [50 mM NaOAc (pH 4.5), 10 mM MgCl₂, 1 M NaCl and 20% ethanol]. The separation of supF tRNA from tRNATYr was monitored by acid urea gel electrophoresis of column fractions followed by RNA blot hybridization. Fractions containing supF tRNA free of tRNA^{Tyr} were pooled.

The purity of all three suppressor tRNAs was greater than 85% as determined by assaying for amino acid acceptor activity and by polyacrylamide gel electrophoresis.

In vitro aminoacylation and isolation of aminoacyl-tRNAs. The U2:A71/U35A36/G72 mutant tRNA^{fMet} (1 A₂₆₀ unit) was aminoacylated with tyrosine in a buffer containing 30 mM Hepes-KOH (pH 7.5), 50 mM KCl, 8 mM MgCl₂, 2 mM DTT, 3 mM ATP, 0.4 mM tyrosine, 0.18 mg/ml BSA, 1 unit of inorganic pyrophosphatase and 20 μg of purified yeast TyrRS (Kowal et al., Proc. Natl. Acad. Sci. USA 98:2268, 2001) in a total volume of 0.4 ml. Aminoacylation of supF tRNA (1 A₂₆₀ unit) was performed in 50 mM Hepes-KOH (pH 7.5), 100 mM KCl, 10 mM MgCl₂, 5 mM DTT, 4 mM ATP, 25 μM tyrosine, 0.18 mg/ml BSA, 1 unit of inorganic pyrophosphatase and 20 units of purified E. coli TyrRS in a total volume of 0.4 ml. Reactions were incubated at 37° C. for 30 min, extracted with phenol equilibrated with 10 mM NaOAc (pH 4.5) and the concentration of NaOAc in the aqueous layer was raised to 0.3 M. The aminoacyl-tRNA was then precipitated with 2 volumes of ethanol. The tRNA was dialyzed against 5 mM NaOAc (pH 4.5), re-precipitated with ethanol, and dissolved in sterile water.

Transfection of COS-1 cells. Cells were cultured in Dulbecco's modified Eagle's medium (DMEM with 4,500 mg/l glucose and 4 mM L-glutamine; Sigma) supplemented with 10% calf serum (Life Technologies), 50 U/mI penicillin and 50 μg/ml streptomycin (both Life Technologies) at 37° C. in a 5% CO₂ atmosphere. 18-24 hours before transfection, cells were subcultured in 12 well dishes (Ø 1.5 cm). Transfection reagent Effectene (Qiagen) was used according to the manufacturer's protocol. Briefly, cells at approximately 30% confluence were transfected with a mixture comprising 1.25 μg of plasmid DNA carrying the reporter gene and 0-5 μg of suppressor tRNA. The mixture of plasmid DNA and tRNA was diluted with EC buffer, supplied by the manufacturer, to a total volume of 50 μl, incubated for 5 min, then mixed with Enhancer (1 μl per μg of total nucleic acids) and incubated for a further 5 min. Effectene (2 μl per μg of total nucleic acids) was added, and the mixture was incubated for 10 min to allow for Effectene-nucleic acid complex formation. All steps above were carried out at room temperature (25° C.). The complexes were diluted with prewarmed (37° C.) DMEM to a total volume of 0.5 ml and added immediately to the cells. 1 ml of medium supplemented with serum and antibiotics was added 6 hours after transfection. Cells were harvested 24-30 hours post-transfection.

Assay for CAT activity. Transfected cells were harvested by adding 0.5 ml of 140 mM NaCl, 20 mM Tris-HCl (pH 7.4), 10 mM EDTA. Cells were then pelleted by centrifugation, resuspended in 30 μl of 0.25 M Tris-HCl (pH 8.0), and lysed by multiple freeze-thaw-cycles. Lysates were clarified by centrifugation, and the protein concentration of the supernatants was determined (BCA protein assay; Pierce) using BSA as standard. 0.5-30 μg of total protein extract in a volume of 20 μl was incubated for 10 min at 65° C. and quick-chilled on ice. The standard reaction (50 μl) contained 20 μl extract, 0.64 mM acetyl coenzyme A, and 1.75 nmol of [¹⁴C]-chloramphenicol (CAM) in 0.5 M Tris-HCl (pH 8.0). After 1 h at 37° C., the reaction was terminated by addition of ethyl acetate and mixing. The ethyl acetate layer was evaporated to dryness, dissolved in ethyl acetate (5 μl) and the solution was applied on to silica gel plates for chromatography with chloroform:methanol (95:5) as the solvent. Following autoradiography, radioactive spots were excised from the plate, and the radioactivity was quantitated by liquid scintillation counting.

Analysis of in vivo state of tRNAs. Total RNAs were isolated from COS1 cells under acidic conditions using TRI-Reagent (Molecular Research Center). tRNAs were separated by acid urea polyacrylamide gel electrophoresis (Varshney et al., J. Biol. Chem. 266:24712, 1991) and detected by RNA blot hybridization using 5 ′-³²P-labeled oligonucleotides.

Results

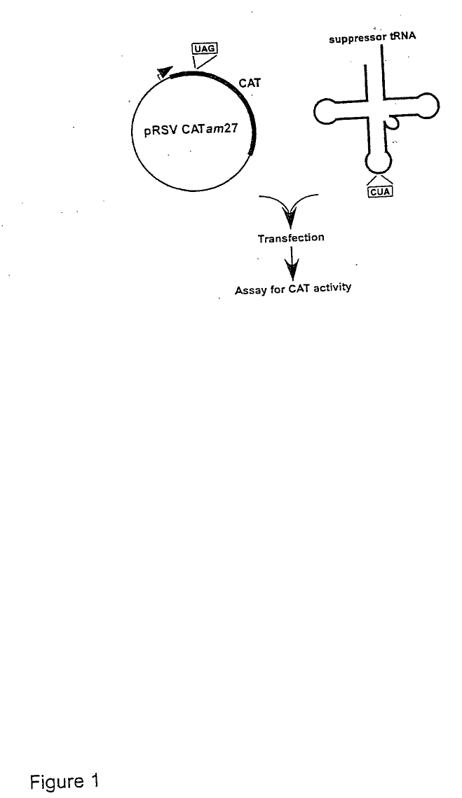

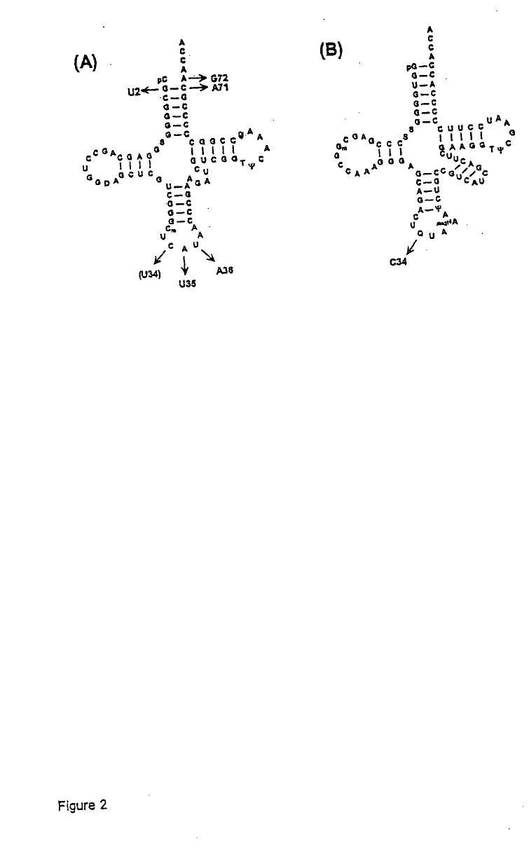

Import of amber suppressor tRNA into mammalian COS1 cells. The assay for import and function of the amber suppressor tRNA (FIG. 1) consisted of co-transfection of COS1 cells with the suppressor tRNA along with the pRSVCATam27 DNA carrying an amber mutation at codon 27 of the chloramphenicol acetyltransferase (CAT) gene followed by measurement of CAT activity in cell extracts. The suppressor tRNA used (FIG. 2A) is derived from the E. coli initiator tRNA^{fMet} and has mutations in the acceptor stem and the anticodon sequence. This tRNA is part of a 21^{st} synthetase-tRNA pair that were developed previously for use in E. coli (Kowal et al., Proc. Natl. Acad. Sci. USA 98:2268, 2001). The G72 mutation in the acceptor stem allows it to act as an elongator tRNA and the U35A36 mutations in the anticodon sequence allow it to read the UAG codon (Seong et al., J. Bio. Chem. 264:6504, 1989). Because the suppressor tRNA contains the C1:G72 base pair, which is one of the critical determinants for eukaryotic TyrRSs, it is aminoacylated in vivo with tyrosine by yeast (Lee et al., Proc. Natl. Acad. Sci. USA 88:11378, 1991; Chow et al., J. Bio. Chem. 268:12855, 1993) and in vitro by human (Wakasugi et al., EMBO J. 17:297, 1998) and COS1 cell TyrRS and is, therefore, expected to be aminoacylated, at least to some extent, with tyrosine in mammalian cells. The tRNA is active in suppression of amber codons in yeast (Lee et al., Proc. Natl. Acad. Sci. USA 88:11378, 1991) and is, therefore, likely to be active in suppression of amber codons in mammalian cells. The tRNA was purified by electrophoresis on 12% polyacrylamide gels and used as such. The methods or reagents used for transfection included electroporation, DEAE-dextran, calcium phosphate, Superfect, Polyfect, Effectene, Lipofectamine, Oligofectamine, or DMRIE-C, a 1:1 (M/M) mixutre of 1,2-dimyristyloxypropyl-3-dimethyl-hydroxyethyl ammonium bromide with cholesterol. No CAT activity was detected in extracts of cells co-transfected using electroporation, DEAE-dextran, calcium phosphate, Superfect or Polyfect. Among the other reagents used, CAT activity was highest (by a factor of >25 fold compared to others) in extracts of cells co-transfected using Effectene (data not shown). The experiments described below for import and function of the suppressor tRNAs were, therefore, all carried out in the presence of Effectene.

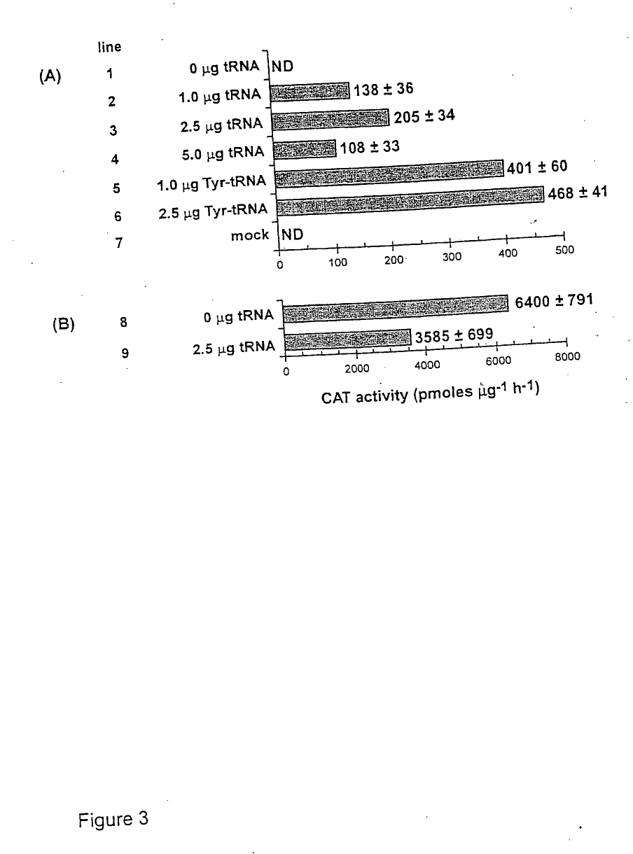

FIG. 3A shows the results of assay for CAT activity in extracts of cells co-transfected with a fixed amount of the pRSVCATam27 plasmid DNA and varying amounts of the suppressor tRNA. Synthesis of CAT requires the presence of the suppressor tRNA during transfection (compare line 1 with lines 2-4). CAT activity reaches a maximum with 2.5 μg of the suppressor tRNA; with 5 μg of the suppressor tRNA, there is a substantial drop in CAT activity (FIG. 3A, lines 3 and 4). This drop in CAT activity is most likely due to an effect of the increased amount of the tRNA on efficiency of co-transfection of the plasmid DNA, since a similar effect of the tRNA is seen on co-transfection of the wild type plasmid DNA (FIG. 3B, lines 8 and 9). The CAT activity in extracts of cells transfected with 2.5 μg of the suppressor tRNA is about 6% of that in cells co-transfected with the wild type pRSVCAT plasmid and the same amount of the suppressor tRNA (FIGS. 3A and B, lines 3 and 9). This is most likely a reflection of the extent of aminoacylation of the suppressor tRNA, the efficiency of amber suppression at this site with the tRNA used and efficiencies of co-transfection of both the plasmid DNA and the suppressor tRNA into COS1 cells.

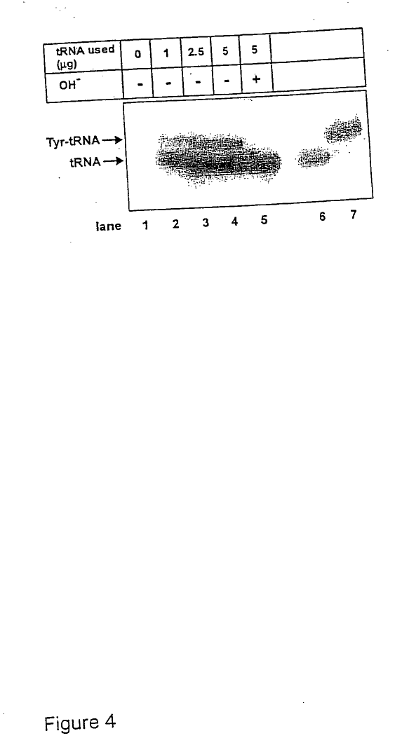

Northern blot analysis shows that only about 8.6% of the suppressor tRNA is aminoacylated in COS I cells (FIG. 4). Thus, aminoacylation of the tRNA is likely one of the factors limiting the extent of suppression of the amber mutation in the CAT gene. Further support for this comes from experiments described below using the ochre suppressor derived from the same tRNA and aminoacylated amber suppressor tRNA.

Import of ochre suppressor tRNA into COS1 cells. The amber suppressor tRNA described above was further mutated in the anticodon (C34 to U34) to generate an ochre suppressor tRNA (FIG. 2A). The import and function of the ochre suppressor tRNA was monitored by co-transfection of COS1 cells with the suppressor tRNA and the pRSVCAToc27 plasmid DNA. Results of experiments carried out in parallel with the ochre and amber suppressor tRNAs show that the ochre suppressor tRNA is about 2-fold more active in suppression of the ochre codon than the amber suppressor tRNA is in suppression of the amber codon (Table 1). This is most likely due to the fact that the ochre suppressor tRNA is a better substrate for yeast and mammalian TyrRS than the amber suppressor tRNA. Both the amber and ochre suppressor tRNAs are specific in suppression of the corresponding codons (Table 1). These results appear, at the outset, to be consistent with the known specificity of amber and ochre suppressors in eukaryotes for the corresponding codons (Capone et al., Mol. Cell. Biol. 6:3059, 1986; Sherman et al. in The Molecular Biology of the Yeast Saccharomyces—Metabolism and Gene Expression (Strathern et al., Eds.), Cold Spring Harbor Laboratory Press, Cold Spring Harbor, N.Y., pp. 463-486, 1982). However, in E. coli, although amber suppressor tRNAs are known to be specific for amber codons, ochre suppressor tRNAs can also read amber codons (Brenner et al., J. Mol. Biol. 13:629, 1965; Eggertsson et al., Microbiol. Rev. 52:354, 1988). Therefore, the finding here that an ochre suppressor tRNA isolated from E. coli is specific for an ochre codon in a mammalian cell is surprising and should be further analyzed. Measurements of CAT activity shown on Table 1 were carried out using 2.5 μg of protein in the COSI cell extracts. Use of ten fold more protein in the assay still failed to detect CAT activity in extracts from cells transfected with the ochre suppressor tRNA along with the pRSVCATam27 plasmid DNA.

TABLE 1
Activities and specifities of amber and ochre suppressor
tRNAs in suppression of amber and ochre codons

| | Suppressor | Micrograms | |
|---|---|---|---|
| Plasmid | tRNA | of tRNA used | CAT activity* |
| pRSVCATam27 | Amber | 0 | ND |
| | | 2.5 | 90.4 ± 7.0 |
| | | 5 | 50.6 ± 5.7 |
| pRSVCAToc27 | Ochre | 0 | ND |
| | | 2.5 | 178.3 ± 10.0 |
| | | 5 | 81.3 ± 10.7 |
| pRSVCATam27 | Ochre | 0 | ND |
| | | 2.5 | ND |
| | | 5 | ND |
| pRSVCAToc27 | Amber | 0 | ND |
| | | 2.5 | ND |
| | | 5 | ND |

COS-1 cells were cotransfected with 1.25 μg of plasmid DNA and suppressor tRNA, as indicated.
CAT activity is defined as picomoles of chloramphenicol acetylated by 1 μg of protein per hour at 37° C.
The values in the table are the average of two independent experiments. Experiments with amber and ochre suppressors were carried out in parallel with a different batch of DMEM and calf serum from that used in FIG. 3. The lower CAT activities with the amber suppressor in these experiments compared to those in FIG. 3 are most likely because of this variation.
ND, not detectable.

Import of aminoacyl-amber suppressor tRNA into COS1 cells. The approach for site-specific insertion of amino acid analogues into proteins requires the import of suppressor tRNA aminoacylated with the amino acid analogue of choice into mammalian cells. In an attempt to determine whether the aminoacyl-linkage in aminoacyl-suppressor tRNA would survive the time and the conditions of transfection needed for import of the suppressor tRNA, the above experiments were repeated with the amber suppressor tRNA that had been previously aminoacylated with tyrosine using yeast TyrRS. Comparison of CAT activity in extracts of cells transfected with the amber suppressor tRNA to that in cells transfected with the amber suppressor Tyr-tRNA shows that at both concentrations of the tRNAs used, CAT activity was significantly higher (2-3 fold) in cells transfected with the Tyr-tRNA (FIG. 3A, compare lines 5 and 6 to lines 2 and 3, respectively). These results demonstrate that an aminoacylated amber suppressor tRNA can withstand the time and the conditions of transfection needed for import into COS1 cells and insert the amino acid attached to the tRNA to a growing polypeptide chain on the ribosome in response to an amber codon.

Import of E. coli supF tRNA into COS1 cells. The approach for site-specific insertion of amino acid analogues into proteins in mammalian cells using the import of suppressor tRNA requires that the suppressor tRNA should not be a substrate for any of the mammalian aaRSs. Otherwise, once the suppressor tRNA has inserted the amino acid analogue at a specific site in the protein, it will be re-aminoacylated with one of the twenty normal amino acids and insert this normal amino acid at the same site. While the amber suppressor tRNA described above proved quite useful for the initial work in determining the conditions necessary for import of both the suppressor tRNA and the reporter plasmid DNA into mammalian cells, the tRNA is a substrate for mammalian TyrRS and is, therefore, not suitable for site-specific insertion of amino acid analogues into proteins in mammalian cells.

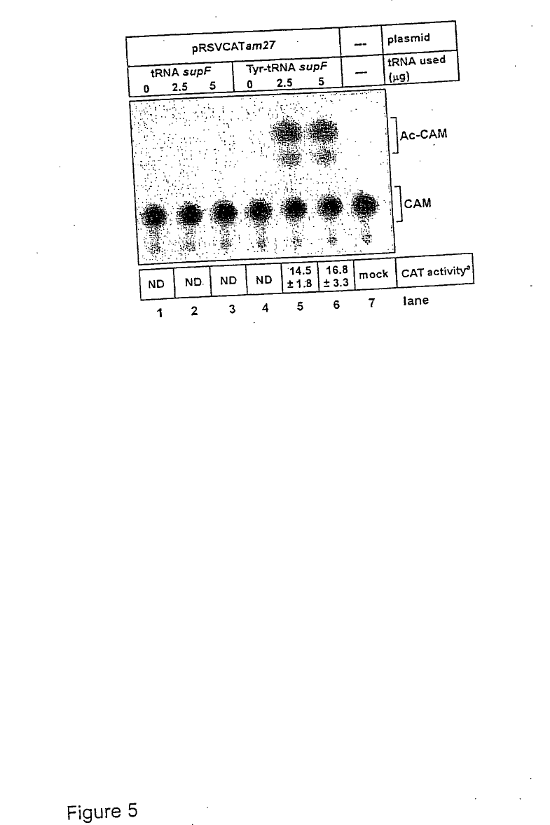

The tRNA selected for this purpose was the E. coli supF tRNA, the amber suppressor tRNA derived from the E. coli tRNA^{Tyr}₁ (FIG. 2B). This tRNA is not a substrate for yeast, rat liver or hog pancreas TyrRS (Clark et al., J. Biol. Chem. 237:3698, 1962; Doctor et al., J. Biol. Chem. 238:3677, 1963) or any of the yeast aaRSs (Edwards et al., Mol. Cell. Biol. 10:1633, 1990). It is also not a substrate for the COS1 cell TyrRS. The SupF tRNA was overproduced in E. coli, purified by column chromatography on BND-cellulose and was aminoacylated with tyrosine using E. coli TyrRS. The SupF tRNA or SupF Tyr-tRNA was co-transfected into COS I cells along with the pRSVCATam27 plasmid DNA and cell extracts were assayed for CAT activity. Extracts of cells co-transfected with up to 5 μg of the supF tRNA had no CAT activity (FIG. 5, lanes 2 and 3). In contrast, extracts of cells co-transfected with the supF Tyr-tRNA had CAT activity (FIG. 5, lanes 5 and 6). These results provide the first indication that an approach involving the import of suppressor tRNA aminoacylated with an amino acid analogue can form the basis of a general method for the site-specific insertion of amino acid analogues into proteins in mammalian cells. The absence of any CAT activity in cells transfected with the supF tRNA shows that this tRNA is not a substrate for any of the mammalian aaRSs and fulfills the requirement described above for the suppressor tRNA to be used for import into mammalian cells.

### Example 2

### Design of a Dual-Luciferase Reporter System and Isolation of HEK293 Cell Lines for Analysis of Amber and Ochre Suppression in Mammalian Cells.

Materials and Methods

Reporter system based on a dual-luciferase fusion protein. A dual-luciferase reporter system was developed based on firefly luciferase (FLuc) and Renilla luciferase (RLuc). The 1.65 kb FLuc gene from pSP-luc+NF (Promega) and the SV40 late poly(A) signal from pGL3-Basic (Promega) were inserted into pBluescript II (SK+) (Stratagene). The 0.95 kb RLuc gene was amplified from pRL-Null (Promega) by PCR using primers designed to introduce a BstEII site in place of the termination codon. This modified RLuc gene was then inserted upstream of the FLuc gene to form the 2.6 kb RLucFLuc fusion (Bennett, M., and Schaack, J., J. Gene Med. 5, 723-732, 2003). Site-directed mutagenesis was used to replace the codon for tyrosine 70 of the wild type FLuc gene with an amber or ochre termination codon to generate RLucFLuc (am70) and RLucFLuc (oc70), respectively. In addition, tyrosine 165 in the RLucFLuc (oc70) gene was replaced by an amber codon to generate RLucFLuc (oc70/am165). The mutant RLucFLuc genes were cloned into the retroviral expression vector pLNCX (Clontech) to generate plasmids pRLucFLuc (oc70), pRLucFLuc (am70), and pRLucFLuc (oc70/am165). These plasmids were then used to establish the following stable HEK293 luciferase cell lines: HEK293-E7 (am70), HEK293-F22 (oc70) and HEK293-D9 (oc70/am165). The stable cell lines were selected on the basis of resistance to geneticin and confirmed by expression of RLuc activity (Bennett, M., and Schaack, J., supra).

Results

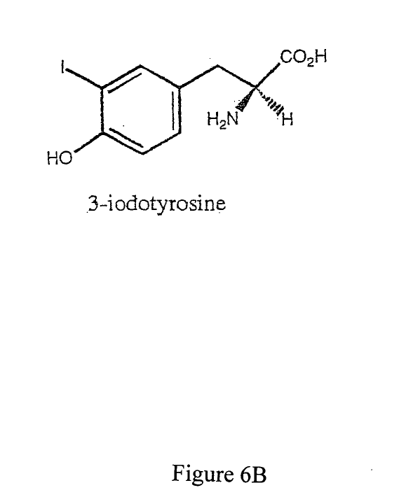

As described above, we showed that import of an aminoacylated amber suppressor tRNA (supF Tyr-tRNA) into mammalian cells by means of transient transfection leads to suppression of an amber codon in the CAT gene. This method provides a general approach to the site-specific incorporation of virtually any unnatural amino acid into a mammalian protein. We designed a highly sensitive reporter system based on a dual-luciferase fusion protein to demonstrate the expansion of this approach to include site-specific insertion of two different unnatural amino acids by combining two termination codons (amber and ochre) in a single mRNA and importing a mixture of amber and ochre suppressor tRNAs into the cell (FIG. 7A).

The DNA sequences encoding firefly luciferase (Photinus pyralis; FLuc) (Wood, K. V., et al., Biochem. Biophys. Res. Commun. 124, 592-596, 1984) and sea pansy luciferase (Renilla reniformis; RLuc) (Matthews, J. C., Hori, K., and Cormier, M. J., Biochemistry 16, 85-91, 1977) were fused to express a single protein with two bioluminescent activities (FIG. 7B). The resulting fusion protein, 865 amino acids long, provides RLuc activity through its N-terminal domain (315 amino acids) and FLuc activity through its C-terminal domain (550 amino acids) (Bennett, M., and Schaack, J., supra). To study the activity of purified suppressor tRNAs imported into mammalian cells, amber and ochre codons were introduced into the FLuc gene to generate plasmids pRLucFLuc (am70), pRLucFLuc (oc70), and pRLucFLuc (oc70/am165) (FIG. 7B). These plasmids were in turn used to establish stable HEK293 luciferase cell lines, HEK293-E7 (am70), HEK293-F22 (oc70) and HEK293-D9 (oc70/am165). The presence of the upstream RLuc gene allowed screening for stable cell lines, based on resistance to geneticin and high RLuc activity in cell extracts. Stable HEK293 luciferase cell lines produced RLuc activities in the range of 1×10⁶ RLU per μg of protein. The RLuc activity could not be used as a common denominator to directly compare the efficiencies of suppression among different cell lines or even different experiments, since the in vivo half-life of the full-length RLucFLuc fusion protein was significantly different from that of the truncated fusion protein consisting of the intact RLuc and 70 amino acids of the FLuc protein (Bennett, M., and Schaack, J., supra). Therefore, results of suppression experiments, in which mixtures of full-length and truncated protein accumulate in the cell, are presented as FLuc activities per μg of total cell protein.

### Example 3

### Concomitant Suppression of Amber and Ochre Codons in COS1 Cells Cotransfected with pRLucFLuc Plasmid and Amber and Ochre Suppressor tRNAs Derived from E. coli Initiator tRNA^{fMet}

Materials and Methods

Plasmids carrying suppressor tRNA genes. Plasmids pRSVCAT/trnfM U2:A71 1U35A36/G72 (Lee, C. P., and RajBhandary, U. L., Proc. Natl. Acad. Sci. USA 88, 11378-11382, 1991) and pRSVCAT/trnfM U2:A71/U34U35A36/G72 (described above and in Köhrer, C., et al., Proc. Natl. Acad. Sci. USA 98, 14310-14315, 2001) contain the genes for amber (fMam) and ochre (fMoc) suppressor tRNAs derived from the E. coli tRNA₂^{fMet}. The plasmid pCDNA1 (Invitrogen) contains the gene for the supF amber suppressor derived from E. coli tRNA₁^{Tyr} (Goodman, H. M., et al., Nature (London) 217, 1019-1024, 1968).

Purification of suppressor tRNAs. Overexpression and purification of the fMam, fMoc and the supF suppressor tRNAs were performed as described in Example 1. The purity of fMam, fMoc and the supF suppressor tRNAs was greater than 90% based on amino acid acceptor activity and polyacrylamide gel electrophoresis.

Transfection of mammalian cells. COS1 cells were cultured in DMEM (with 4,500 mg/L of glucose and 4 mM glutamine; Sigma) supplemented with 10% fetal bovine serum (Atlanta Biologicals Inc.), 50 units/ml of penicillin and 50 μg/ml of streptomycin (Invitrogen) at 37° C. in a 5% CO₂ atmosphere. HEK293 cell lines were maintained in the medium described above supplemented with 250 μg/ml of geneticin (Invitrogen). Eighteen to twenty hours before transfection, cells were subcultured into 12-well dishes. Transfection of COS1 and HEK293 cells with tRNA and/or plasmid DNA using Effectene (Qiagen) was as described above. The amount of suppressor tRNA used per transfection was adjusted according to tyrosine acceptance which reflects the amount of ‘active’ suppressor tRNA present per sample. A non-suppressing tRNA (tRNA^{fMet}) was used to keep the amount of total tRNA constant throughout the transfection experiments.

Assay for luciferase activity. The Dual-Luciferase Reporter System (DLR; Promega) was used to measure FLuc and RLuc activities in mammalian cell extracts. 15-24 h post-transfection, the medium was removed and cells were washed twice with PBS. 200 μl of 1× Passive Lysis Buffer (PLB; supplied by the manufacturer) was added per well and cells were lysed for 15 min at room temperature with gentle shaking. Lysates were clarified by centrifugation and the supernatants were immediately analyzed as follows. 20 μl of Luciferase Assay Reagent II (LAR II) was added to 2-4 μl of lysate, and firefly luciferase activity was read. Quenching of the FLuc signal and concomitant activation of RLuc were performed by adding 20 μl of Stop & Glo Reagent. Measurement of luciferase activities was carried out on a Sirius tube luminometer (Berthold Detection Systems). For standard DLR assays, a 10-second pre-measurement delay and a 15-second measurement period were programmed. Luciferase activities are given as relative luminescence units (RLU) per μg of total cell protein, the values shown in the Tables represent the averages of at least three independent experiments. The protein concentration of cell lysates was determined with a BCA protein assay (Pierce) using BSA as standard.

Results

As described above, we showed that amber (fMam) and ochre (fMoc) suppressor tRNAs derived from the E. coli initiator tRNA (tRNA^{fMet}) could be imported into mammalian cells and suppressed amber and ochre mutations, respectively, at position 27 of the CAT gene. Both of these tRNAs, fMam and fMoc, are substrates for yeast and mammalian TyrRS and are aminoacylated with tyrosine by mammalian cell extracts. Here, we have asked whether these two tRNAs can be used for concomitant suppression of two different termination codons located in the FLuc coding region.

COS1 cells were co-transfected with the pRLucFLuc (oc70/am165) plasmid and purified fMam and fMoc suppressor tRNAs. Cells were harvested after 24 hours and extracts assayed for FLuc activity. Cells transfected with a mixture of amber and ochre suppressor tRNAs have substantial amounts of FLuc activity (87.1×10³ RLU per μg of protein; Table 2, line 1). Cells transfected with fMam tRNA alone have essentially no FLuc activity, indicating that this tRNA is unable to translate the ochre codon at position 70 of the reporter mRNA (Table 2, line 2). Cells transfected with fMoc tRNA alone display a low level of FLuc activity (Table 2, compare lines 1 and 3), suggesting that the tRNA also reads the amber codon at position 165 but only weakly. Extracts from cells transfected with pRLucFLuc (oc70/am165) and a non-suppressing control tRNA (tRNA^{fMet}; Table 2, line 4) yield background of less than 0.5% of maximum FLuc activity.

Our results described in Example 1 showed that the fMoc suppressor tRNA did not suppress the amber codon in the CATam27 reporter gene. Our finding that this tRNA can suppress an amber codon in the FLuc mRNA, albeit weakly, is most likely due to the superior sensitivity of the firefly luciferase assay compared to assay for CAT activity.

TABLE 2
Concomitant suppression of amber and ochre codons in COS1 cells.
COS1 cells were transfected with a mixture of 2.5 μg of pRLucFLuc
(oc70/am165) plasmid DNA and fMam and fMoc suppressor tRNAs
as indicated.

| | fMam | fMoc | tRNA^{fMet(a)} | FLuc activity | Relative |
|---|---|---|---|---|---|
| | (μg) | (μg) | (μg) | ×10³ (RLU/μg) | FLuc activity |
| 1 | 2.5 | 2.5 | — | 87.1 ± 8.4 | 100% |
| 2 | 2.5 | — | 2.5 | 0.6 ± 0.1 | 0.7% |
| 3 | — | 2.5 | 2.5 | 3.2 ± 0.2 | 3.7% |
| 4 | — | — | 5.0 | 0.4 ± 0.1 | 0.5% |
| ^{(a)}E. coli initiator tRNA^{fMet} was added to keep the amount of total tRNA constant at 5 μg. In control experiments that were performed in parallel, transient transfection of 2.5 μg of plasmid carrying the wild type RLucFLuc fusion gene yielded FLuc activities of 1.1-1.2 × 10⁶ RLU/μg. FLuc activities obtained in line 1 reflecting the combined suppression of both | | | | | |
| # the amber and ochre codon thereby correspond to a suppression level of ˜8%. This would indicate that the amber and ochre codons are each suppressed to the level of ˜28%. | | | | | |

### Example 4

### Concomitant Suppression of Amber and Ochre Codons in the Stable HEK293-D9 (oc70/am165) Luciferase Cell Line by fMam and fMoc Suppressor tRNAs

Materials and Methods

Transfection and assay for luciferase activity were performed as described in Example 3.

Results

The use of the HEK293-D9 (oc70/am165) luciferase cell line allowed, for the first time, to monitor directly the uptake into mammalian cells of suppressor tRNAs instead of mixtures of reporter plasmid DNA and tRNA, thereby facilitating optimization of transfection conditions for importing mixtures of amber and ochre suppressor tRNAs. Initially, the ratio of fMam and fMoc tRNA was kept at 1:1 and the total amount of suppressor tRNA at 5 μg. FLuc activity from 2.5 μg each of amber and ochre suppressor tRNA is 255×10³ RLU per μg of protein (Table 3, line 1). Consistent with previous experiments, fMam suppressor tRNA is highly specific for suppressing amber codons (Table 3, line 2), whereas fMoc suppressor tRNA shows a low level (3.7%) of non-specific read-through activity of the amber codon (Table 3, line 3).

Keeping the amount of fMoc tRNA constant at 2.5 μg and increasing the amount of fMam to 5 μg increases FLuc activity from 210 to 305×10³ RLU/μg of protein while maintaining high specificity (Table 3, compare line 5 to lines 6 and 7). This increase in FLuc activity suggests that the amount of aminoacylated amber suppressor tRNA is limiting when added at a 1:1 ratio of amber:ochre suppressor tRNA. Consequently, adjusting the ratio of amber and ochre suppressor tRNAs used for transfection allows optimal protein expression with minimal non-specific read-through of amber codons by ochre suppressor tRNAs.

In a similar experiment, fMam tRNA was kept at 2.5 μg and the amount of fMoc tRNA was increased to 5 μg (Table 3, compare line 5 to lines 8 and 9). This results in only a small increase in FLuc activity from 210 to 231 RLU/μg of protein, suggesting that the ochre suppressor tRNA is not limiting. At higher concentrations of fMoc tRNA and in the absence of fMam tRNA, there is increased read-through of the amber codon from 3.7 to 12.6% (Table 3, compare lines 3 and 9).

TABLE 3
Concomitant suppression of amber and ochre codons in a stable
HEK293 luciferase cell line. HEK293-D9 (oc70/am165) cells were
transfected with a mixture of fMam and fMoc suppressor tRNA
as indicated.

| | fMam | fMoc | tRNA^{fMet(a)} | FLuc activity | Relative |
|---|---|---|---|---|---|
| | (μg) | (μg) | (μg) | ×10³ (RLU/μg) | FLuc activity |
| 1 | 2.5 | 2.5 | — | 255 ± 94.5 | 100% |
| 2 | 2.5 | — | 2.5 | 0.4 ± 0.11 | 0.2% |
| 3 | — | 2.5 | 2.5 | 9.4 ± 1.10 | 3.7% |
| 4 | — | — | 5.0 | 0.01 ± 0.01 | <0.1% |
| 5 | 2.5 | 2.5 | 2.5 | 210 ± 24.5 | 100% |
| 6 | 5.0 | 2.5 | — | 305 ± 37.2 | 145% |
| 7 | 5.0 | — | 2.5 | 0.6 ± 0.07 | 0.3% |
| 8 | 2.5 | 5.0 | — | 231 ± 27.3 | 110% |
| 9 | — | 5.0 | 2.5 | 26.5 ± 4.05 | 12.6% |
| 10 | — | — | 7.5 | 0.01 ± 0.01 | <0.1% |

^{(a)}E. coli initiator tRNA^{fMet} was added to keep the amount of total tRNA constant at 5 μg (lines 1-4) and 7.5 μg (lines 5-10), respectively.

### Example 5

### Identification, Purification and Import of an Ochre Suppressor tRNA (supC.A32) that is not Aminoacylated by Mammalian aaRSs

Materials and Methods

Plasmids carrying suppressor tRNA genes. The plasmid pCDNA1 (Invitrogen) contains the gene for the supF amber suppressor derived from E. coli tRNA₁^{Tyr} (Goodman, et al., supra). A 329 bp fragment carrying the gene for supF tRNA including its original promoter and transcription termination signals was amplified by PCR and inserted into the BamHI site of pRSVCATam27 (Capone, J. P., et al., Mol. Cell. Biol. 6, 3059-3067, 1986). which carries the gene for chloramphenicol acetyltransferase (CAT) with an amber mutation at position 27, to generate pRSVCATam27/supF. In attempts to construct the ochre suppressor supC, the supF gene was mutagenized to introduce a C34 to U34 change in the anticodon of the tRNA using site-directed mutagenesis. No clones carrying the wild type supC tRNA could be isolated, likely due to toxicity of overexpression of supC tRNA in E. coli (Altman, S., et al., J. Mol. Biol. 56, 195-197, 1971). Instead, a supC tRNA mutant with a C32 to A32 mutation (supC.A32), which was found to be active as an ochre suppressor in E. coli, was isolated. Position 27 of the CAT reporter gene was changed from an amber to an ochre codon to generate pRSVCAToc27/supC.A32.

Purification of suppressor tRNAs. The supC.A32 ochre suppressor tRNA was isolated from E. coli strain CA274 [lacZ125(am) trp49(am) relA1 spoT1] carrying the plasmid pRSVCAToc27/supC.A32 and purified by benzoylated-naphthoylated DEAE-cellulose column chromatography. Separation of supC.A32 tRNA from wild type tRNA^{Tyr} was monitored by acid urea gel electrophoresis of column fractions followed by RNA blot hybridization using 5′-³²P-labeled oligonucleotides (Varshney, U., et al., J. Biol. Chem. 266, 24712-24718, 1991). Fractions containing supC.A32 tRNA free of tRNA^{Tyr} were pooled. The purity of supC.A32 tRNA was 45-50%.

In vitro aminoacylation and isolation of aminoacyl-tRNAs. Aminoacylation of supF and supC.A32 tRNA was carried out as described in Example 1 on 1 A₂₆₀ unit of tRNAs using purified E. coli tyrosyl-tRNA synthetase (TyrRS). Aminoacylation of tRNAs was essentially quantitative as analyzed by acid urea gel electrophoresis followed by RNA blot hybridization (Varshney, supra).

Results

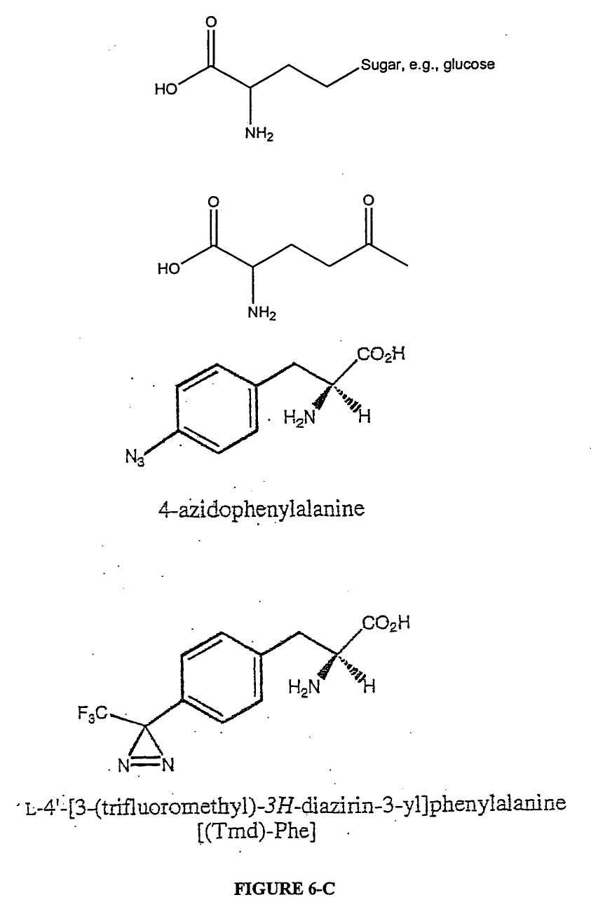

While the amber and ochre suppressor tRNAs described above were important for establishing the feasibility of concomitant suppression of two different termination codons in a single mRNA, they are aminoacylated by mammalian TyrRS and therefore unsuitable for site-specific insertion of unnatural amino acids into proteins in mammalian cells. As described in Example 1, we demonstrated that the E. coli supF tRNA (FIG. 8A) is not a substrate for any of the mammalian aaRSs and fulfills all of the requirements for its use in site-specific insertion of unnatural amino acids. Here, we asked whether an ochre suppressor (supC) derived from the same tRNA would also not be a substrate for mammalian aaRSs and whether it would specifically suppress ochre codons in mammalian cells. To generate the supC ochre suppressor tRNA (Altman, supra), the anticodon sequence of supF tRNA was mutagenized to U34U35A36. Attempts to isolate supC tRNA by site-specific mutagenesis of the supF tRNA gene only yielded supC tRNA mutants that carried additional mutations in the anticodon stem-loop region, likely due to toxicity caused by overexpression of ochre suppressor tRNAs in E. coli (Altman, supra; Eggertsson, G., and Söll, D. Microbiol. Rev. 52, 354-374, 1988). One of the mutants, supC.A32 (FIG. 8B), was selected based on its ability to suppress the ochre codon in a CAT reporter gene and on the level of the suppressor tRNA overproduction in E. coli (data not shown).

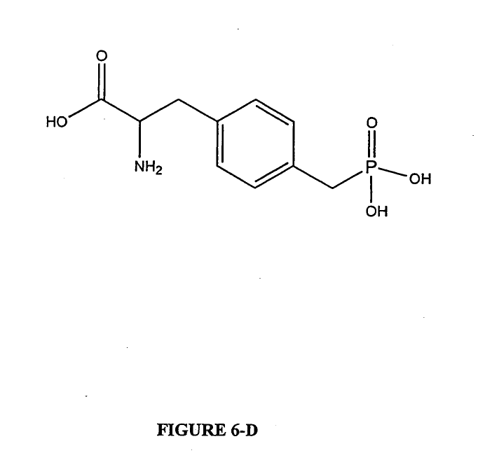

The supF and supC.A32 suppressor tRNAs were expressed in E. coli, purified (see Materials and Methods) and aminoacylated in vitro with tyrosine using E. coli TyrRS (FIG. 9). The supF tRNA or supF Tyr-tRNA and supC tRNA or supC Tyr-tRNA were then transfected into HEK293-E7 (am70) and HEK293-F22 (oc70) cells, which carry a single termination codon at position 70 of the FLuc coding region. Extracts of cells transfected with suppressor tRNA without prior aminoacylation have essentially no FLuc activity (Table 4, lines 1, 3, 6 and 8). In contrast, extracts from HEK293-E7 (am70) cells transfected with supF Tyr-tRNA (line 2) and HEK293-F22 (oc70) cells transfected with supC.A32 Tyr-tRNA (line 7) yield FLuc activities of 52.2×10³ and 50.9×10³ RLU/μg of protein, respectively. These results demonstrate that the supC.A32 ochre suppressor tRNA is also not aminoacylated by any of the mammalian aaRSs. Thus, to the best of our knowledge, supC.A32 tRNA represents the first “orthogonal” ochre suppressor tRNA that has been described.

The specificity of these amber and ochre suppressor tRNAs was analyzed by transfecting HEK293-E7 (am70) cells with supC.A32 Tyr-tRNA and HEK293-F22 (oc70) cells with supF Tyr-tRNA. Consistent with previous results, supC.A32 tRNA also translates the amber codon to a certain extent (11%; Table 4, line 4), whereas supFtRNA is highly specific for amber codons (Table 4, line 9).

TABLE 4
Import of supF and supC.A32 tRNA into stable HEK293 luciferase
cell lines. HEK293-E7 (am70) and HEK293-F22 (oc70) cells were
transfected with supF and supC.A32 tRNA with and without
prior aminoacylation as indicated.

| | | |
|---|---|---|
| | FLuc activity | Relative |
| Suppressor | ×10³ | FLuc |
| tRNA^{(a)} | (RLU/μg) | activity |

HEK293-E7 (am70)

| | | |
|---|---|---|
| 1 supF^{(b)} | 0.8 ± 0.34 | 1.5% |
| 2 Tyr-supF | 52.2 ± 5.35 | 100% |
| 3 supC.A32^{(b)} | 0.8 ± 0.01 | 1.5% |
| 4 Tyr-supC.A32 | 5.8 ± 0.32 | 11.1% |
| 5 mock | 0.4 ± 0.03 | 0.8% |

HEK293-F22 (oc70)

| | | |
|---|---|---|
| 6 supC.A32^{(b)} | 0.4 ± 0.02 | 0.8% |
| 7 Tyr-supC.A32 | 50.9 ± 2.93 | 100% |
| 8 supF^{(b)} | 0.01 ± 0.02 | <0.1% |
| 9 Tyr-supF | 0.01 ± 0.02 | <0.1% |
| 10 mock | 0.01 ± 0.01 | <0.1% |

^{(a)}HEK293 cells were transfected with 3.75 μg of active suppressor tRNA as indicated. tRNA^{fMet} was added to keep the amount of total tRNA constant at 10 μg. All experiments were carried out in triplicates, except those marked with ^{(b)} which were done in duplicates.

### Example 6

### Concomitant Suppression of Amber and Ochre Codons in HEK293-D9 (oc70/am165) Cells by Import of Aminoacylated Amber (supF) and Ochre (supC.A32) Suppressor tRNAs

Materials and Methods

tRNA purification and aminoacylation and transfection of mammalian cells were performed as described above.

Results

HEK293-D9 (oc7O/am ]65) cells were transfected with a mixture of aminoacylated supF Tyr-tRNA and supC.A32 Tyr-tRNA. To ensure high specificity of supF and supC.A32 tRNA for their respective termination codons, the ratio of amber:ochre suppressor tRNA was adjusted to 2:1. Cells transfected with both of the suppressor tRNAs produce significant amounts of FLuc activity, 47.8×10³ RLU per μg of protein (Table 5, line 1). Cells transfected with supF Tyr-tRNA alone have essentially no FLuc activity (Table 5, line 2) whereas cells transfected with supC.A32 Tyr-tRNA alone had 3.4% of the maximum FLuc activity obtained with both tRNAs (Table 5, line 3). No FLuc activity is detected upon import of supF and supC.A32 tRNA without prior aminoacylation (data not shown).

TABLE 5
Import of supF Tyr-tRNA and supC.A32 Tyr-tRNA leads to concomitant
suppression of amber and ochre codons in a stable HEK293 luciferase
cell line. HEK293-D9 (oc70/am165) cells were transfected with 5 μg
of supF Tyr-tRNA (Tyr-supF) and 2.5 μg of supC.A32 Tyr-tRNA
(Tyr-supC.A32) as indicated.

| | Tyr-supF^{(a)} | Tyr-supC.A32^{(a)} | FLuc activity | Relative |
|---|---|---|---|---|
| | (μg) | (μg) | ×10³ (RLU/μg) | FLuc activity |
| 1 | 5 | 2.5 | 47.8 ± 4.55 | 100% |
| 2 | 5 | — | 0.1 ± 0.00 | 0.2% |
| 3 | — | 2.5 | 1.6 ± 0.40 | 3.4% |
| 4 | — | — | 0.05 ± 0.05 | 0.1% |

^{(a)}HEK293 cells were transfected with active suppressor tRNA as indicated. tRNA^{fMet} was added to keep the amount of total tRNA constant at 10 μg.

These results clearly illustrate that both supF and supC.A32 tRNA fulfill the basic requirements for site-specific incorporation of unnatural amino acids into proteins in a mammalian system. They also confirm that the import of a mixture of amber and ochre suppressor tRNAs into mammalian cells followed by the concomitant suppression of amber and ochre codons can form the basis of a general approach to site-specific insertion of two different unnatural amino acids into the same protein or into different proteins.

While the supF amber and supC.A32 ochre suppressor tRNAs suppress, respectively, amber and ochre codons and supF tRNA is specific for the amber codon, the supC.A32 tRNA like other ochre suppressor tRNAs in E. coli (Brenner, S., and Beckwith, J. R., J. Mol. Biol. 13, 629-637, 1965; Eggertsson, G., and Söll, D. Microbiol. Rev. 52, 354-374, 1988) also reads the amber codon, although to a limited extent (11% in Table 4; 3.4% in Table 5). As in all known cases in translation, this non-specific readthrough of the amber codon is likely to be much less in the presence of the cognate amber suppressor tRNA. For example, Tirrell and coworkers have shown that in the presence of an orthologous tRNA^{Phe}_{AAA} that is expressed and aminoacylated with an amino acid analogue in E. coli, the UUU phenylalanine codon, normally translated by the resident tRNA^{Phe} with the GAA anticodon, is now almost exclusively translated by the tRNA^{Phe}_{AAA} (Kwon, I., et al., J. Am. Chem. Soc. 125, 7512-7513, 2003). Even if the non-specific readthrough of the amber codon by the ochre codon remains at a level of 11%, which is extremely unlikely, this should not affect the potential applications of the double-suppression approach for the synthesis and the uses, described above, of proteins carrying two different fluorescent amino acids or two different phosphono-amino acids. For example, if the ochre suppressor tRNA delivers the same fluorescent amino acid to the site of the amber codon and the ochre codon, a small fraction of the protein will have the same fluorescent amino acid at two positions in the reporter protein. This should not interfere with intramolecular FRET between two different fluorescent amino acids on the rest of the reporter protein.

Finally, in contrast to bacterial ochre suppressor tRNAs, eukaryotic ochre suppressor tRNAs are specific for the ochre codon (Capone, J. P., et al., Mol. Cell. Biol. 6, 3059-3067, 1986; Sherman, F. Suppression in the yeast Saccharomyces cerevisiae. in: The Molecular Biology of the Yeast Saccharomyces—Metabolism and Gene Expression, eds. Strathern, J. N., Jones, E. W. & Broach, J. R. (Cold Spring Harbor Laboratory Press, Cold Spring Harbor, N.Y.), pp. 463-486, 1982; Laski, F. A., et al., EMBO J. 3, 2445-2452, 1984). Therefore, in addition to the supC.A32 ochre suppressor tRNA used here, eukaryotic ochre suppressor tRNAs that are not aminoacylated by mammalian aaRSs will constitute an excellent source of ochre suppressor tRNAs for the site-specific insertion of two different unnatural amino acids into proteins in mammalian cells.

### Example 7

### A Complete Set of Orthogonal Amber, Ochre and Opal Suppressor tRNAs Derived from E. coli tRNA^{Gln} (hsup2am, hsup2oc, hsup2op)

Materials and Methods

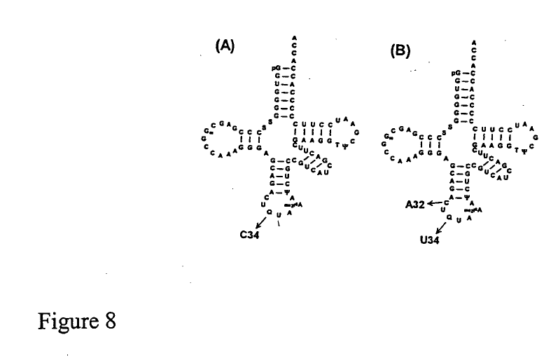

Plasmids. This section describes plasmids used in Examples 7-11. The dual-luciferase reporter system coding for the Renilla luciferase (Renilla reniformis; RLuc) and firefly luciferase (Photinus pyralis; FLuc) fusion protein has been described above. The DNA sequences encoding Renilla and firefly luciferase were fused to express a single protein with two bioluminescent activities (FIG. 11). Plasmid pRF.wt was used to express a fusion protein that provides RLuc activity through its N-terminal domain and FLuc activity through its C-terminal domain. Site-specific mutagenesis (Quikchange; Stratagene) was performed to introduce amber, ochre and opal codons into the FLuc coding region to generate plasmids pRF.Y70am, pRF.Y70oc, pRF.Y70op, pRF.Q162am, pRF.Q162oc, pRF.Q162op, pRF.Y165am, pRF.Q283op, pRF.Y70oc/Y165am, pRF.Y70op/Y165am and pRF.Y70oc/Y165am/Q283op. In addition, tyrosine 70, glutamine 162 and tyrosine 165 of the wild type FLuc gene were replaced with glutamine and serine codons, respectively, to yield plasmids pRF.Y70Q, pRF.Y70S, pRF.Q162S, pRF.Y165Q and pRF.Y165S.

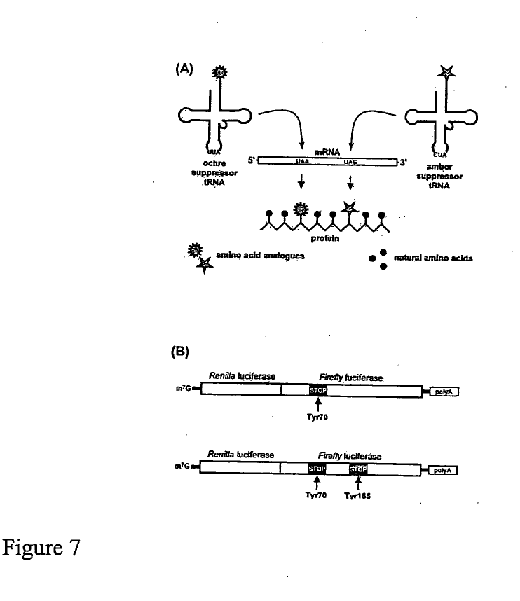

Plasmid pSVB.hsup2am contains the gene for the hsup2am amber suppressor tRNA derived from the E. coli tRNA^{Gln} (FIG. 10; Drabkin, H. J., et al., Mol. Cell. Biol., 16, 907-913, 1986). This tRNA was previously called hsup2A9am. Ochre (hsup2oc) and opal (hsup2op) suppressor tRNAs were generated by introducing C34 to U34 and C34U35 to U34C35 changes, respectively, in the anticodon of the tRNA. Plasmids pSVB.hsup2am, pSVB.hsup2oc and pSVB.hsup2op were altered to introduce additional U32 to C32, C38 to A38 or U32C38 to C32A38 mutations. Plasmids carrying amber, ochre and opal suppressor tRNAs derived from the human serine tRNA (pSVB.hseram, pSVB.hseroc, pSVB.hserop) have been described before (Capone, J. P., et al., EMBO J., 4, 213-221, 1985).

Suppressor tRNA genes hsup2am, hsup2oc, hsup2.C32A38am and hsup2.C32A38oc were cloned into pBAD-araC (Invitrogen) for inducible expression of suppressor tRNAs in E. coli. The tRNA genes were amplified by PCR (forward primer: 5′-GGGGCCATGGACCAATTTGTTGGGGTATAGCCAAGCGGTAAGG-3′ (SEQ ID NO: 3); reverse primer: 5′-GGGGTACGTATTGAATAAATTGGCTGGGGTACGAGG-3′ (SEQ ID NO: 4)) using the respective pSVB plasmids as templates. The ˜110 bp PCR fragment was cut with NcoI and SnaBI and ligated into pBAD-araC cut with the same enzymes.

The 1.7 kb DNA fragment encoding E. coli GlnRS was amplified by PCR (forward primer: 5′-CCCGAATTCGCCACCATGCATCACCATCACCATCACAGTG AGGCAGAAGCCC-3′ (SEQ ID NO: 5); reverse primer: 5+-CCCGCGGCCGCTTACTCGCCTACTTTCGCCC-3′ (SEQ ID NO: 6)) from pESC-LEU.GlnRS (Kowal, A. K., et al., Proc. Natl. Acad. Sci. U.S.A., 98, 2268-2273, 2001) and inserted into the EcoRI/NotI sites of pCMVTNT (Promega). The resulting plasmid pTNT.EcGlnRS allows expression of E. coli GlnRS in mammalian cells with a His6-tag at the N-terminus of the protein.

Transfection of mammalian cells. HEK293T cells were maintained in DMEM (with 4,500 mg/L of glucose; Cellgro) supplemented with 10% fetal bovine serum (Atlanta Biologicals Inc.), 2 mM glutamine, 100 units/ml of penicillin and 100 μg/ml of streptomycin (Invitrogen) at 37° C. in a 5% CO₂ atmosphere. Eighteen to twenty hours before transfection, cells were subcultured into 24-well plates. Transfection of HEK293T cells with plasmid DNA using Effectene (Qiagen) was as described above, with minor modifications. Briefly, cells at approx. 60-70% confluence were co-transfected with 0.5 μg of pRF plasmid carrying the luciferase reporter gene, 0.5 μg of pSVB plasmid carrying the tRNA gene and 5-10 ng of pCMVTNT plasmid carrying the E. coli GlnRS gene. The mixture of plasmid DNAs was diluted in 25 μl of EC buffer, supplied by the manufacturer, and then mixed with 2.5 μl Enhancer and 5 μl Effectene. The complexes were diluted with 0.25 ml of prewarmed (37° C.) DMEM and added to the cells. 0.275 ml of medium supplemented with 10% serum and 10 mM sodium butyrate (Sigma) was added 3 hours after transfection. Cells were harvested 48 hours post-transfection.

Assay for luciferase activity. The Dual-Luciferase Reporter System (DLR; Promega) was used to measure luciferase activities in mammalian cell extracts as described above and in (Köhrer, C., et al, Chem. Biol., 10, 1095-1102, 2003). Measurement of luciferase activities was carried out on a Sirius tube luminometer (Berthold Detection Systems). For standard DLR assays, a 10-second pre-measurement delay and a 15-second measurement period were programmed. Luciferase activities are given as relative luminescence units (RLU) per μg of total cell protein. The protein concentration of cell lysates was determined with a BCA protein assay (Pierce) using BSA as standard. The values shown in the Tables and Figures represent the averages of at least three independent experiments; variations among experiments were less than 15%.

Analysis of in vivo state of tRNAs. Total RNAs were isolated from mammalian cells under acidic conditions using TRI-Reagent (Sigma) or TRIzol (Invitrogen). tRNAs were separated by acid urea polyacrylamide gel electrophoresis (Varshney, U., et al., J. Biol. Chem., 266, 24712-24718 1991), electroblotted onto Hybond-N+membrane (Amersham) and detected by RNA blot hybridization. Membranes were prehybridized at 42° C. in 10× Denhardt's solution/6×SSC/0.5% SDS. Hybridization was performed at 30° C. in 6×SSC/0.1% SDS in the presence of a 5 ′-³²P-labeled oligonucleotide, complementary to nucleotides 57-72 of the hsup2am tRNA. A 5′-³²P-labeled oligonucleotide complementary to nucleotides 7-22 of the human serine tRNA was also used as an internal standard. Membranes were washed at room temperature, once with 6×SSC/0.1% SDS followed by two washes with 6×SSC, and then subjected to autoradiography. Northern blots were quantified by PhosphorImager analysis using ImageQuant software (Molecular Dynamics).

Results

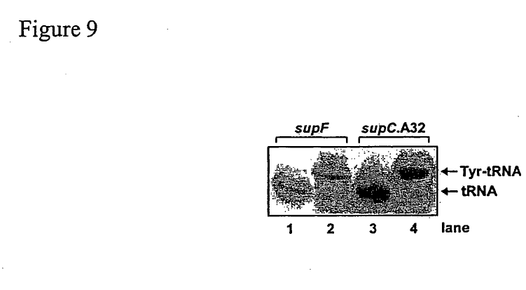

We described above the expression of an amber suppressor tRNA derived from E. coli tRNA^{Gln} in mammalian cells (FIG. 10). The E. coli suppressor tRNA gene, flanked by the original 5′ and 3′ sequences of the human initiator tRNA^{Met}, was cloned into the mammalian expression vector pSVBpUC. An additional C9 to A9 mutation was introduced to improve transcription efficiency by mammalian RNA polymerase III. The resulting plasmid was transfected into mammalian cells, and the tRNA hsup2A9am (which we rename here hsup2am for the sake of simplicity) was expressed along with or without E. coli GlnRS. The data presented above indicated clearly that the suppressor tRNA was active in COS1 cells; see also FIG. 12, lanes 1 & 2). Furthermore, its activity as an amber suppressor was strictly dependent upon co-expression of E. coli GlnRS; see also Table 6, lines 1-3). This work provided the first example of an orthogonal suppressor tRNA in mammalian cells.

In this example, we describe the generation of ochre and opal suppressor tRNAs derived from E. coli tRNA^{Gln} by changing the anticodon of the hsup2am tRNA to UUA (ochre; hsup2oc) and UCA (opal; hsup2op), respectively. To test and compare the activities of hsup2am, hsup2oc and hsup2op tRNAs in suppression, plasmids carrying amber, ochre or opal stop codon mutations in codon 162 of the firefly luciferase (FLuc) gene (FIG. 11) were transfected into HEK293T cells along with plasmids carrying the genes for the suppressor tRNAs and E. coli GlnRS. Cells were harvested 48 hours post-transfection and extracts were assayed for luciferase activity. Table 6 summarizes the results. No FLuc activity is detected over background in HEK293T cells that express the suppressor tRNAs but do not contain E. coli GlnRS (Table 6; lines 2, 5 and 8). Thus, along with the hsup2am, the hsup2oc and hsup2op tRNAs are also not recognized by any of the endogenous native mammalian aaRSs. Suppression of the amber, ochre and opal codon in the FLuc gene was only observed in the presence of E. coli GlnRS (Table 6; lines 3, 6, and 9) yielding FLuc activities of 0.79×10⁶ RLU/μg, 0.024×10⁶ RLU/μg and 0.044×10⁶ RLU/μg, respectively. These data represent the first example of a complete isogenic set of orthogonal amber, ochre and opal suppressor tRNAs and provide the first report of a 21^{st} synthetase-ochre suppressor tRNA pair suitable for expression in mammalian cells.

Interestingly, hsup2am tRNA yielded significantly higher levels of FLuc activity, approximately 20-30 fold over the hsup2oc and hsup2op tRNAs, with the ochre suppressor having the lowest activity. These striking differences in suppression efficiencies can be explained, at least partly, by more efficient in vivo aminoacylation of the amber suppressor tRNA by E. coli GlnRS, as shown by acid urea PAGE followed by RNA blot hybridization of total tRNA isolated from HEK293T cells using a probe directed against nucleotides 57-72 of the tRNA (FIG. 12). PhosphorImager analysis indicates that hsup2am tRNA is aminoacylated almost quantitatively (˜87%), whereas hsup2oc and hsup2op tRNAs are aminoacylated to lower levels, ˜32% and ˜45%, respectively. These findings are not completely surprising since nucleotides in the anticodon of E. coli tRNA^{Gln}, changed to generate these suppressor tRNAs, are generally believed to be critical recognition elements for E. coli GlnRS based on the crystal structure of the tRNA^{Gln}-GlnRS complex (Rould, M. A., et al., Science, 246, 1135-1142, 1989) and on biochemical studies (Jahn, M., et al., Nature, 352, 258-260, 1991). An additional faster migrating band was detected for hsup2oc and hsup2op tRNAs. These bands probably represent tRNA species with additional modifications in the anticodon-loop of the suppressor tRNA (e.g. U34) or conformational variants of the tRNA. The effect of base modifications on the mobility of tRNAs in acid urea PAGE has been described before (Mangroo, D., et al., J. Bacteriol., 177, 2858-2862, 1995).

TABLE 6
Amber, ochre and opal suppression in HEK293T cells.

| | | | | FLuc activity |
|---|---|---|---|---|
| line | RLucFLuc | tRNA | aaRS | (RLU/μg) |
| 1 | Q162am | — | — | 1,487 |
| 2 | Q162am | hsup2am | — | 1,311 |
| 3 | Q162am | hsup2am | QRS | 786,668 |
| 4 | Q162oc | — | — | 1,350 |
| 5 | Q162oc | hsup2oc | — | 913 |
| 6 | Q162oc | hsup2oc | QRS | 24,065 |
| 7 | Q162op | — | — | 9,871 |
| 8 | Q162op | hsup2op | — | 6,108 |
| 9 | Q162op | hsup2op | QRS | 42,735 |

HEK293T cells were co-transfected with 0.5 μg of pRF plasmid carrying the appropriate luciferase reporter gene, 0.5 μg of pSVB plasmid carrying the tRNA gene and 5 ng (hsup2am, hsup2oc)-10 ng (hsup2op) of pCMVTNT plasmid carrying the E. coli GlnRS gene. Transfection of 0.5 μg of plasmid carrying the wild type RLucFLuc fusion gene yielded FLuc activities of 82.7 × 10⁶ RLU/μg.

### Example 8

### Mutants of the Orthogonal Amber, Ochre and Opal Suppressor tRNAs with Enhanced Suppressor Activity in Mammalian Cells

Materials and Methods

Immunoblot analysis. Cell lysates were prepared as described above and concentrated by acetone precipitation. Proteins were resolved by SDS/PAGE, transferred onto Immobilon PVDF membrane (Millipore) and probed with primary antibodies against FLuc (AB3256; polyclonal; Chemicon), RLuc (MAB4410; monoclonal; Chemicon) and actin (sc-9104; monoclonal; Santa Cruz Biotechnologies). The horseradish peroxidase-conjugated secondary antibodies were anti-goat IgG (Promega), anti-mouse IgG and anti-rabbit IgG (both Amersham). Signals were visualized using enhanced oxidase/luminol reagents (ECL; Perkin Elmer Life Sciences).

Additional materials and methods were described above.

Results

The activity and aminoacylation specificity of tRNAs is affected by sequences in and around the anticodon loop and stem and by base modifications, especially those in the anticodon loop (Yarus, M. Science, 218, 646-652, 1982; Yarus, M., et al., J. Biol. Chem., 261, 496-505, 1986; Agris, P. F., Nucleic Acids Res., 32, 223-238, 2004; Colby, D. S., et al., Cell, 9, 449-463, 1976). To improve the activity of hsup2 derived tRNAs, we introduced the following mutations in the anticodon loop of the hsup2am, hsup2oc and hsup2op tRNA genes (FIG. 10): mutation of U38 to A38 to generate a potential recognition motif for the dimethylallyl diphosphate:tRNA dimethylallyl transferase (DMAPP-transferase); mutation of U32 to C32; and a double mutation of U32 and U38 to C32 and A38, respectively. DMAPP-transferase is encoded by the miaA gene and has been identified in E. coli, yeast and mammalian cells. This enzyme is responsible for modifying the A37 residue, which is believed to be important for the suppressor activity of tRNAs by strengthening the interaction between codon and anticodon (Ericson, J. U. and Björk, G. R. J. Mol. Biol., 218, 509-516, 1991; Björk, G. R. Biosynthesis and function of modified nucleosides. In Söll D., and RajBhandary U. L. (eds.), tRNA: Structure, Biosynthesis, and Function. American Society for Microbiology, Washington DC, pp.1 65-205, 1995, the entirety of which is incorporated herein by reference). The minimum recognition motif on the tRNA consists of a stretch of three A's, A36-A37-A38 (summarized in Motorin, Y., et al., RNA, 3, 721-733, 1997). The C32A38 double mutation generates an anticodon loop sequence which mimics the sequence found in most strong suppressor tRNAs from prokaryotic and eukaryotic sources (Drabkin, supra; Yarus, supra; Smith, D., et al., Nucleic Acids Res., 15, 4669-4686, 1987). The C32 mutation also removes a potential transcription termination signal (a string of 4 U residues U32-U35) for RNA polymerase III in the hsup2oc tRNA (Koski, R. A., et al., Cell, 22, 415-425, 1980; Hamada, M., et al., J. Biol. Chem., 275, 29076-29081, 2000).

The FLuc activities in extracts of cells transfected with the various mutants derived from hsup2am are shown in Table 7. The hsup2/C32am tRNA yielded FLuc activities of 2.3×10⁶ RLU/μg, representing a ˜3 fold increase of activity compared to the hsup2am tRNA. The A38 mutation resulted in a ˜15 fold increase of FLuc activity, whereas the combined C32 and A38 mutations resulted in a 36 fold increase of FLuc activity. Similarly, the FLuc activities for the hsup2oc mutants (Table 8) increased 3.9 and 6 fold for the C32 and A38 single mutants, respectively. The most striking effects were seen for the hsup2/C32A38oc and hsup2/C32A38op double mutants. The hsup2/C32A38oc mutant showed an activity of 3.76×10⁶ RLU/μg corresponding to a 156 fold increase from the original hsup2oc tRNA. The FLuc activity in cells transfected with the mutant hsup2op tRNA also increased from 0.04×10⁶ to 8.57×10⁶ RLU/μg for the hsup2/C32A38op double mutant (Table 9, lines 3 and 5) corresponding to a 200 fold increase. Altogether, these mutants provide an isogenic set of amber, ochre and opal suppressor tRNAs, each with a range of suppression activities in mammalian cells.

The sequences of suppressor tRNAs derived from E. coli tRNA^{Gln} are presented below and are an aspect of the invention. The anticodon is indicated in bold; mutations at positions 32 and 38 of the tRNA are underlined.

Amber suppressor tRNAs:
hsup2am

(SEQ ID NO: 7)

5′-UGGGGUAUAGCCAAGCGGUAAGGCACCGGAUUCUAAUUCCGGCAUUC
CGAGGUUCGAAUCCUCGUACCCCAG-3′
hsup2/C32am

(SEQ ID NO: 8)

5′-UGGGGUAUAGCCAAGCGGUAAGGCACCGGACUCUAAUUCCGGCAUUC
CGAGGUUCGAAUCCUCGUACCCCAG-3′
hsup2/A38am

(SEQ ID NO: 9)

5′-UGGGGUAUAGCCAAGCGGUAAGGCACCGGAUUCUAAAUCCGGCAUUC
CGAGGUUCGAAUCCUCGUACCCCAG-3′
hsup2/C32A38am

(SEQ ID NO: 10)

5′-UGGGGUAUAGCCAAGCGGUAAGGCACCGGACUCUAAAUCCGGCAUUC
CGAGGUUCGAAUCCUCGUACCCCAG-3′
Ochre suppressor tRNAs:
hsup2oc

(SEQ ID NO: 11)

5′-UGGGGUAUAGCCAAGCGGUAAGGCACCGGAUUUUAAUUCCGGCAUUC
CGAGGUUCGAAUCCUCGUACCCCAG-3′
hsup2/C32oc

(SEQ ID NO: 12)

5′-UGGGGUAUAGCCAAGCGGUAAGGCACCGGACUUUAAUUCCGGCAUUC
CGAGGUUCGAAUCCUCGUACCCCAG-3′
hsup2/A38oc

(SEQ ID NO: 13)

5′-UGGGGUAUAGCCAAGCGGUAAGGCACCGGAUUUUAAAUCCGGCAUUC
CGAGGUUCGAAUCCUCGUACCCCAG-3′
hsup2/C32A3 8oc

(SEQ ID NO: 14)

5′-UGGGGUAUAGCCAAGCGGUAAGGCACCGGACUUUAAAUCCGGCAUUC
CGAGGUUCGAAUCCUCGUACCCCAG-3′
Opal suppressor tRNAs
hsup2op

(SEQ ID NO: 15)

5′-UGGGGUAUAGCCAAGCGGUAAGGCACCGGAUUUCAAUUCCGGCAUUC
CGAGGUUCGAAUCCUCGUACCCCAG-3′
hsup2/C32A38op

(SEQ ID NO: 16)

5′-UGGGGUAUAGCCAAGCGGUAAGGCACCGGACUUCAAAUCCGGCAUUC
CGAGGUUCGAAUCCUCGUACCCCAG-3′

All of the suppressor tRNA mutants, including those with highest suppression activities still require E. coli GlnRS for their activity (Tables 7, 8 and 9) and are, therefore, completely orthogonal in HEK293T cells. The 21^{st} synthetase-amber, ochre and opal suppressor tRNA pairs composed of the strongest C32A38 double mutants, and E. coli GlnRS had translational efficiencies of 35%, 4.5% and 10.4%, respectively, as estimated by normalizing FLuc activities in cells transfected with the mutant RLucFLuc genes to those in cells transfected with the wild type RLucFLuc gene (Tables 7, 8, and 9). These efficiencies compare favorably to those obtained with the homologous human serine amber, ochre and opal suppressor tRNAs (22.4%, 6.1% and 27.8%, respectively), which are aminoacylated by the endogenous native human seryl-tRNA synthetase (Table 10).

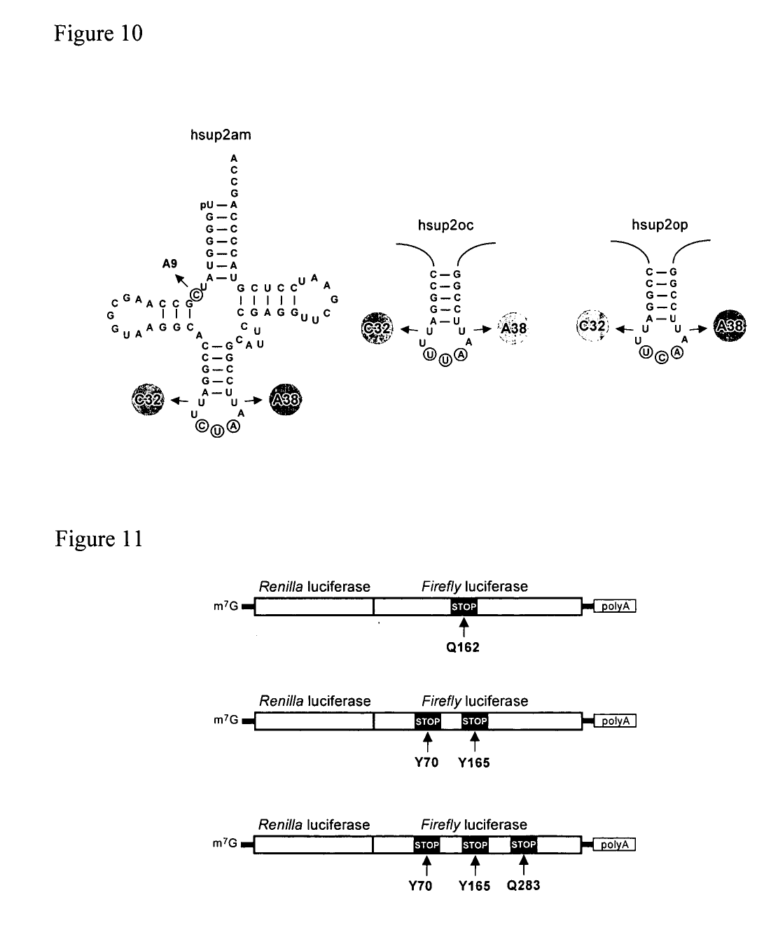

The results of immunoblot analyses using anti-FLuc antibodies (FIG. 13) also confirm the orthogonality of the enhanced amber, ochre and opal suppressor tRNAs. Thus, an ˜87 kDa protein corresponding to the full length RLucFLuc fusion protein is detected only in cells cotransfected with plasmids carrying the genes encoding the reporter protein, the suppressor tRNA and E. coli GlnRS. Furthermore, the intensities of the full length RLucFLuc fusion protein band parallel the luciferase activities in enzyme assays, providing additional evidence for the translational efficiencies of the E. coli tRNA^{Gln} derived suppressor tRNAs in the order amber>opal>ochre.

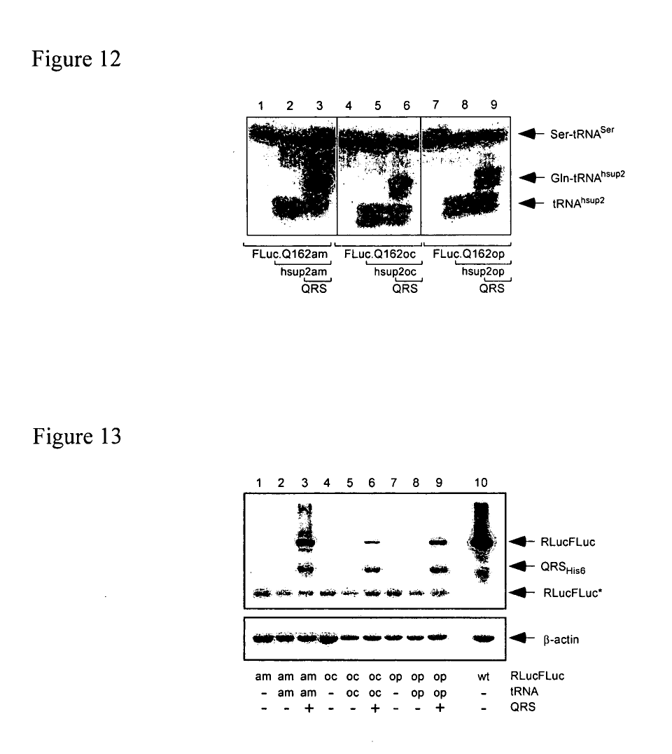

The increased FLuc activities in cells transfected with the various mutant suppressor tRNAs could be due to a combination of increased steady state level of the tRNAs, increased extent of aminoacylation of the tRNAs and/or increased ribosomal activity of the tRNAs in suppression. To distinguish among these possibilities, the steady-state levels and extent of aminoacylation of all mutant tRNAs were analyzed by acid urea PAGE followed by RNA blot hybridization using probes directed against the mutant tRNAs and human tRNA₃^{Ser} as an internal control (FIG. 14). The extent of aminoacylation remained essentially the same for all hsup2am mutants (87-95%), the appearance of an additional faster migrating band suggests heterogeneity in base modifications or the occurrence of conformational variants in some of the mutants (FIG. 14A). Both the A38 mutation and the C32 mutation had similar effects. The extent of aminoacylation increased from ˜32% for hsup2oc and hsup2/A38oc tRNAs to ˜50% for hsup2/C32oc and hsup2/C32A38oc tRNAs (FIG. 14B), whereas the extent of aminoacylation of the opal suppressor tRNA remained essentially unaltered at ˜50% (FIG. 14C). In general, the relative intensity of the faster migrating band seemed to increase for all the C32A38 double mutants. Comparison of the total signals obtained for the suppressor tRNAs to that obtained for the human tRNA₃^{Ser} showed a maximal variation in steady state levels of 2-2.3 fold for some of the mutant tRNAs indicating a higher expression level or greater stability of these tRNAs. Taken together, these results suggest that the increased FLuc activities of 36, 156 and 200 fold seen in cells transfected with the C32A38 mutants of the hsup2am, hsup2oc and hsup2op tRNAs, respectively, are primarily due to increased activity of these tRNAs in suppression at the ribosomal level.

Thus, without wishing to be bound by any theory, we suggest that the primary reason for the increased activity of the E. coli tRNA^{Gln} derived amber, ochre and opal suppressors also carrying the C32A38 mutations, in mammalian cells is most likely increased activity at the ribosomal level. For example, in the case of the amber suppressor tRNA, where there is a 36 fold increase in activity of the most active mutant, the tRNAs are all aminoacylated to approximately the same levels (87-95 %) and there is at the most a 2-2.5 fold difference in steady state levels of the suppressor tRNAs (FIG. 14A). Similarly, for the mutants derived from the ochre and opal suppressor tRNAs, while there is approximately a 1.5 fold difference in extent of aminoacylation of one of the tRNAs (FIGS. 14B and 14C) and a 2-2.5 fold difference in steady state levels of some of the tRNAs, these differences cannot account for the 156 and 200 fold increase in activity, respectively, of the ochre and opal suppressor tRNAs carrying the C32A38 mutations.

The orthogonality of the tRNA^{Gln} derived ochre and opal suppressors was not necessarily expected. In particular, the opal suppressor tRNA, which has C35 in the middle of the anticodon sequence, could have been a substrate for one of the mammalian aaRSs, including TrpRS, which uses C35 as an important identity determinant. For example, in E. coli, Söll, Inokuchi and coworkers (Rogers, M. J., et al., Proc. Natl. Acad. Sci. U.S.A., 89, 3463-3467, 1992) have shown that the E. coli tRNA^{Gln} derived opal suppressor is a substrate for E. coli TrpRS and that this opal suppressor tRNA inserts predominantly tryptophan into proteins. Our finding that the E. coli tRNA^{Gln} derived opal suppressor tRNA is not a substrate for mammalian TrpRS (Table 4) or any other mammalian aaRS (FIG. 5C), indicates that the requirements in the substrate tRNA for mammalian TrpRS are quite different from those of E. coli TrpRS.

In bacteria, ochre suppressor tRNAs also suppress amber codons (Brenner, S. and Beckwith, J. R. J. Mol. Biol., 13, 629-637, 1965; Raftery, L. A., et al., Egan, J. B., Cline, S. W. and Yarus, M. (1984) Defined set of cloned termination suppressors: In vivo activity of isogenetic UAG, UAA, and UGA suppressor tRNAs. J. Bacteriol., 158, 849-859, 1984; Eggertsson, supra), whereas in eukaryotes, to the extent that they have been studied, ochre suppressor tRNAs are specific for the ochre codon (6,9,45). This is commonly ascribed to Wobble pairing (46) between the modified U, the first nucleotide in the anticodon of the ochre suppressor tRNA and G, the third nucleotide of the amber codon UAG. Nevertheless, the finding in this work that the most active E. coli tRNA^{Gln} derived ochre suppressor, when expressed in mammalian cells, is still specific for the ochre codon (Table 6) is noteworthy, since the same tRNA, when expressed in E. coli, suppresses the amber codon quite well in E. coli (FIG. 6). Whether the specificity of the ochre suppressor tRNA, expressed in mammalian cells, is due to a different base modification of the U at the Wobble position 34 or whether the eukaryotic ribosome is inherently more restrictive in translation of the amber codon by an ochre suppressor tRNA, remains to be seen.

TABLE 7
Amber suppression in HEK293T cells.

| | | | | FLuc activity | Translation* | |
|---|---|---|---|---|---|---|
| line | RLucFLuc | tRNA | aaRS | (RLU/μg) | efficiency (%) | fold increase |
| 1 | Q162am | — | — | 1,487 | 0.00 | — |
| 2 | Q162am | hsup2am | — | 1,311 | 0.00 | — |
| 3 | Q162am | hsup2am | QRS | 786,668 | 0.95 | 1.00 |
| 4 | Q162am | hsup2/C32am | — | 1,680 | 0.00 | — |
| 5 | Q162am | hsup2/C32am | QRS | 2,319,895 | 2.81 | 2.95 |
| 6 | Q162am | hsup2/A38am | — | 3,516 | 0.00 | — |
| 7 | Q162am | hsup2/A38am | QRS | 11,761,149 | 14.22 | 14.95 |
| 8 | Q162am | hsup2/C32A38am | — | 30,030 | 0.04 | — |
| 9 | Q162am | hsup2/C32A38am | QRS | 28,510,124 | 34.48 | 36.24 |
| 10 | wt | — | — | 82,683,171 | 100.00 | — |

HEK293T cells were co-transfected with 0.5 μg of pRF plasmid carrying the luciferase reporter gene, 0.5 μg of pSVB plasmid carrying the tRNA gene and 5 ng of pCMVTNT plasmid carrying the E. coli GlnRS gene.
*Translational efficiency as estimated by normalizing FLuc activities in cells transfected with the mutant RLucFLuc genes to FLuc activities in cells transfected with the wild type RLucFLuc gene.

TABLE 8
Ochre suppression in HEK293T cells.

| | | | | FLuc activity | Translation | |
|---|---|---|---|---|---|---|
| line | RLucFLuc | tRNA | aaRS | (RLU/μg) | efficiency* (%) | fold increase |
| 1 | Q162oc | — | — | 1,350 | 0.00 | — |
| 2 | Q162oc | hsup2oc | — | 913 | 0.00 | — |
| 3 | Q162oc | hsup2oc | QRS | 24,065 | 0.03 | 1.00 |
| 4 | Q162oc | hsup2/C32oc | — | 1,258 | 0.00 | — |
| 5 | Q162oc | hsup2/C32oc | QRS | 91,387 | 0.11 | 3.80 |
| 6 | Q162oc | hsup2/A38oc | — | 1,137 | 0.00 | — |
| 7 | Q162oc | hsup2/A38oc | QRS | 144,919 | 0.18 | 6.02 |
| 8 | Q162oc | hsup2/C32A38oc | — | 2,108 | 0.00 | — |
| 9 | Q162oc | hsup2/C32A38oc | QRS | 3,755,288 | 4.54 | 156.05 |
| 10 | wt | — | — | 82,683,171 | 100.00 | — |

HEK293T cells were co-transfected with 0.5 μg of pRF plasmid carrying the luciferase reporter gene, 0.5 μg of pSVB plasmid carrying the tRNA gene and 5 ng of pCMVTNT plasmid carrying the E. coli GlnRS gene.
*Translational efficiency as estimated by normalizing FLuc activities in cells transfected with the mutant RLucFLuc genes to FLuc activities in cells transfected with the wild type RLucFLuc gene.

TABLE 9
Opal suppression in HEK293T cells.

| | | | | FLuc activity | Translation | |
|---|---|---|---|---|---|---|
| line | RLucFLuc | tRNA | aaRS | (RLU/μg) | efficiency* (%) | fold increase |
| 1 | Q162op | — | — | 9,871 | 0.01 | — |
| 2 | Q162op | hsup2op | — | 6,108 | 0.01 | — |
| 3 | Q162op | hsup2op | QRS | 42,735 | 0.05 | 1.00 |
| 4 | Q162op | hsup2/C32A38op | — | 9,063 | 0.01 | — |
| 5 | Q162op | hsup2/C32A38op | QRS | 8,565,996 | 10.36 | 200.44 |
| 6 | wt | — | — | 82,683,171 | 100.00 | — |

HEK293T cells were co-transfected with 0.5 μg of pRF plasmid carrying the luciferase reporter gene, 0.5 μg of pSVB plasmid carrying the tRNA gene and 10 ng of pCMVTNT plasmid carrying the E. coli GlnRS gene.
*Translational efficiency as estimated by normalizing FLuc activities in cells transfected with the mutant RLucFLuc genes to FLuc activities in cells transfected with the wild type RLucFLuc gene.

TABLE 10
Activity of amber, ochre and opal suppressor tRNAs derived from
the human serine tRNA (hseram, hseroc and hserop) in HEK293T cells.

| | | | FLuc activity | Translation |
|---|---|---|---|---|
| line | RLucFLuc | tRNA | (RLU/μg) | efficiency* (%) |
| 1 | Q162am | hseram | 13,350,146 | 22.43 |
| 2 | Q162oc | hseroc | 3,648,376 | 6.13 |
| 3 | Q162op | hserop | 16,525,712 | 27.76 |
| 4 | Q162S | — | 59,531,883 | 100.00 |

HEK293T cells were co-transfected with 0.5 μg of pRF plasmid carrying the luciferase reporter gene, 0.5 μg of pSVB plasmid carrying the tRNA gene and 5 ng of pCMVTNT plasmid. Transfection of 0.5 μg of plasmid carrying the wild type RLucFLuc fusion gene yielded FLuc activities of 82.7 × 10⁶ RLU/μg.
*Translational efficiency as estimated by normalizing FLuc activities in cells transfected with the mutant RLucFLuc genes to FLuc activities in cells transfected with mutant RLucFLuc.Q162S gene.

### Example 9

### Specificity of hsup2/C32A38am, hsup2/C32A38oc and hsup2/C32A38op tRNAs for Their Cognate Codons

Materials and Methods

Expression of mutant suppressor tRNAs in E. coli. Transformants of E. coli CA274 (HfrH lacZ125am trpEam) carrying pBAD.hsup2am, pBAD.hsup2oc, pBAD.hsup2/C32A38am and pBAD.hsup2/C32A38oc, respectively, were grown in LB_{Amp} medium at 37° C. to mid-log phase (A₆₀₀ of 0.5-0.6). Arabinose was added to a final concentration of 0.002% to induce transcription from the P_{BAD} promoter. Cells were then grown for 80 minutes at 37° C. and two more hours at room temperature (˜20° C.), harvested by centrifugation, and analyzed for β-galactosidase activity using the Beta-Glo assay system (Promega). Relative β-galactosidase activities were normalized to the specific activities of β-lactamase in the same extract (Mayer, C., et al., Biochemistry, 42, 4787-4799, 2003) and to cell density at time of harvest.

Additional materials and methods were described above.

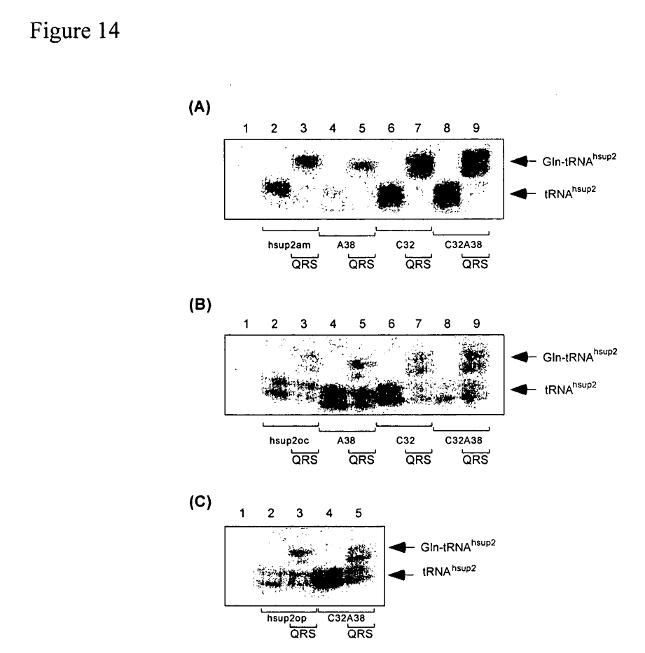

Results. The specificity of hsup2/C32A38am, hsup2/C32A38oc and hsup2/C32A38op tRNAs towards their cognate codons was investigated using the pRF.Q162am, Q162oc and Q162op reporter genes (Table 11). Despite their greatly enhanced activities towards their cognate codons, each suppressor tRNA translated only the corresponding cognate codon and had no significant activity towards a non-cognate stop codon. These results were confirmed with different luciferase stop codon mutations (at positions Y70, S163 and Y165) in different codon contexts (data not shown). The specificity of ochre suppressor tRNA mutants for the ochre codon in mammalian cells is in striking contrast to results obtained in E. coli. For example, expression of the same hsup2oc and hsup2/C32A38oc tRNAs in E. coli CA274 leads to significant suppression of an amber mutation in the chromosomal β-galactosidase gene (FIG. 15) by the ochre suppressor tRNAs.

TABLE 11
Specificity of amber, ochre and opal suppression in HEK293T cells.

| | | | | FLuc activity |
|---|---|---|---|---|
| line | RLucFLuc | tRNA | aaRS | (RLU/μg) |
| 1 | Q162am | — | — | 1,487 |
| 2 | Q162am | hsup2/C32A38am | QRS | 28,510,124 |
| 3 | Q162am | hsup2/C32A38oc | QRS | 22,009 |
| 4 | Q162am | hsup2/C32A38op | QRS | 1,818 |
| 5 | Q162oc | — | — | 1,350 |
| 6 | Q162oc | hsup2/C32A38am | QRS | 1,396 |
| 7 | Q162oc | hsup2/C32A38oc | QRS | 3,755,288 |
| 8 | Q162oc | hsup2/C32A38op | QRS | 2,632 |
| 9 | Q162op | — | — | 9,871 |
| 10 | Q162op | hsup2/C32A38am | QRS | 16,589 |
| 11 | Q162op | hsup2/C32A38oc | QRS | 7,366 |
| 12 | Q162op | hsup2/C32A38op | QRS | 8,565,996 |

HEK293T cells were co-transfected with 0.5 μg of pRF plasmid carrying the luciferase reporter gene, 0.5 μg of pSVB plasmid carrying the tRNA gene and 5 ng (hsup2am, hsup2oc)-10 ng (hsup2op) of pCMVTNT plasmid carrying the E. coli GlnRS gene. Transfection of 0.5 μg of plasmid carrying the wild type RLucFLuc fusion gene yielded FLuc activities of 82.7 × 10⁶ RLU/μg.

### Example 10

### Concomitant Suppression of Two Different Termination Codons (Amber and Ochre; Amber and Opal) in RLucFLuc mRNAs

Materials and Methods were described above.

Results

The hsup2/C32A38am and hsup2/C32A38oc tRNAs were used for concomitant suppression of amber and ochre codons using the RLucFLuc.Y70ocY165am reporter gene that had been used in earlier experiments described above. Table 12 summarizes the data. Co-expression of the hsup2/C32A38am and the hsup2/C32A38oc tRNAs and E. coli GlnRS in HEK293T cells resulted in a significant level of FLuc activity, 2.6×10⁶ RLU/μg (Table 12; line 3). This level of FLuc activity is similar to that found in cells co-expressing the amber and ochre suppressors derived from human serine tRNA (Table 12; line 6). As expected, no FLuc activity was detected in cells not expressing E. coli GlnRS (line 2) or only one of the suppressor tRNAs (lines 4 and 5).

Similarly, the hsup2/C32A38am and hsup2/C32A38op tRNAs were used for concomitant suppression of amber and opal codons using the RLucFLuc.Y70opY165am reporter gene. In this case also, coexpression of the two tRNAs resulted in a significant level of FLuc activity of 1.7×10⁶ RLU/μg (Table 12, line 9). Little activity was detected in cells not expressing E. coli GlnRS (line 8) or only one of the suppressor tRNAs (lines 10 and 11). These results clearly show that the newly generated amber, ochre and opal suppressor tRNAs derived from E. coli tRNA^{Gln}, hsup2/C32A38am, hsup2/C32A38oc, and hsup2/C32A38op, fulfill the requirements of high activity and specificity for their cognate codons necessary for site-specific incorporation of one or two unnatural amino acids into proteins in a mammalian system.

TABLE 12
Concomitant suppression of amber & ochre codons and amber & opal codons in
HEK293T cells

| | | | | | FLuc activity | Translation |
|---|---|---|---|---|---|---|
| line | RLucFLuc | tRNA | tRNA | aaRS | (RLU/μg) | efficiency* (%) |
| 1 | Y70ocY165am | — | — | — | 447 | — |
| 2 | Y70ocY165am | C32A38am | C32A38oc | — | 121 | — |
| 3 | Y70ocY165am | C32A38am | C32A38oc | QRS | 2,657,349 | 1.98 |
| 4 | Y70ocY165am | — | C32A38oc | QRS | 1,435 | — |
| 5 | Y70ocY165am | C32A38am | — | QRS | 1,611 | — |
| 6 | Y70ocY165am | hseram | hseroc | — | 3,247,888 | 2.43 |
| 7 | Y70opY165am | — | — | — | 361 | — |
| 8 | Y70opY165am | C32A38am | C32A38op | — | 35,030 | — |
| 9 | Y70opY165am | C32A38am | C32A38op | QRS | 1,721,225 | 1.29 |
| 10 | Y70opY165am | — | C32A38op | QRS | 428 | — |
| 11 | Y70opY165am | C32A38am | — | QRS | 5,830 | — |
| 12 | Y70opY165am | hseram | hserop | — | 3,177,332 | 2.37 |
| 13 | wt | — | — | — | 133,917,640 | 100.00 |

HEK293T cells were co-transfected with 0.5 μg of pRF plasmid carrying the luciferase reporter gene, 0.5 μg of pSVB plasmid carrying the tRNA gene (each) and 10 ng of pCMVTNT plasmid carrying the E. coli GlnRS gene. 3 hours post-transfection, cells were fed with fresh medium containing 10% serum, 10 mM sodium butyrate (see Experimental Procedures) and 2 mM glutamine.
*Translational efficiency as estimated by normalizing FLuc activities in cells transfected with the mutant RLucFLuc genes to FLuc activities in cells transfected with the wild type RLucFLuc gene.

### Example 11

### Concomitant Suppression of Three Different Termination Codons in RLucFLuc mRNA

Materials and Methods were described above.

Results. The availability of a complete set of orthogonal amber, ochre and opal suppressor tRNAs enabled us to ask whether it would be possible to concomitantly suppress three different termination codons in a mRNA. Accordingly, the E. coli tRNA^{Gln} derived amber, ochre and opal suppressors were transfected into HEK293T cells along with the RLucFLuc.Y70ocY165amQ283op reporter gene. In a parallel experiment, human serine amber, ochre and opal suppressor tRNAs were also used. Table 13 summarizes the data on FLuc activities in extracts of transfected cells. It can be seen that the E. coli tRNA^{Gln} derived amber, ochre and opal suppressors can suppress all three termination codons in the reporter mRNA (Table 13, line 3). Suppression is dependent upon expression of E. coli GlnRS (Table 13, compare lines 2 and 3) and upon the presence of all three suppressor tRNAs (data not shown). As expected, FLuc activity is lower when suppressor tRNAs are used to suppress three different termination codons instead of two (compare FLuc activity in Table 13, line 3 to Table 12, line 3).

FLuc activity in extracts of cells transfected with all three E. coli tRNA^{Gln} derived suppressors is about 25% of that obtained with the human tRNA^{Ser} derived suppressors (Table 13, compare lines 3 and 4). One possible reason for this is that the E. coli GlnRS activity in transfected cells becomes limiting, particularly since these suppressor tRNAs are known to be poor substrates for E. coli GlnRS (Jahn, M., et al., Nature, 352, 258-260, 1991); see also FIG. 14) and now three glutamine-accepting suppressor tRNAs are overexpressed to significant levels while E. coli GlnRS remains constant throughout the experiment. In contrast, the anticodon sequences in the human tRNA^{Ser} derived suppressors are not important for their aminoacylation by human seryl-tRNA synthetase (Achsel, T. and Gross, H. J. EMBO J., 12, 3333-3338, 1993; Heckl, M., et al., FEBS Lett., 427, 315-319, 1998). Thus, it may well be possible to increase the efficiency of suppression of ochre and opal codons by increasing the levels of expression of E. coli GlnRS in transfected cells, e.g., by utilizing a stronger promoter as described elsewhere herein. Another possibility would be to use mutant forms of E. coli GlnRS that have increased activity towards suppressor tRNAs (see, e.g., Kobayashi, T., et al., Nat. Struct. Biol., 10, 425-432, 2003).

TABLE 13
Concomitant suppression of amber, ochre and opal codons in HEK293T cells.

| | | | | | | FLuc activity | Translation |
|---|---|---|---|---|---|---|---|
| line | RLucFLuc | tRNA | tRNA | tRNA | aaRS | (RLU/μg) | efficiency* (%) |
| 1 | oc/am/op | — | — | — | — | 26 | — |
| 2 | oc/am/op | C32A38am | C32A38oc | C32A38op | — | 256 | — |
| 3 | oc/am/op | C32A38am | C32A38oc | C32A38op | QRS | 49,987 | 0.03 |
| 4 | oc/am/op | hseram | hseroc | hserop | — | 214,150 | 0.11 |
| 5 | wt | — | — | — | — | 188,000,547 | 100.00 |

HEK293T cells were co-transfected with 0.5 μg of pRF plasmid carrying the luciferase reporter gene pRF.Y70ocY165amQ283op (oc/am/op), 0.5 μg of pSVB plasmid carrying the tRNA gene (each) and 10 ng of pCMVTNT plasmid carrying the E. coli GlnRS gene. 3 hours post-transfection, cells were fed with fresh medium containing 10% serum, 10 mM sodium butyrate (see Experimental Procedures) and 2 mM glutamine.
*Translational efficiency as estimated by normalizing FLuc activities in cells transfected with the mutant RLucFLuc genes to FLuc activities in cells transfected with the wild type RLucFLuc gene.

### Example 12

### Amber, Ochre and Opal Suppressor tRNAs derived from E. coli tRNA^{Gln}, Suppress UAG, UAA and UGA Termination Codons

Materials and Methods. HEK293T cells were transfected with plasmids carrying the genes for hsup2/C32A38am, hsup2/C32A38oc and hsup2/C32A38op tRNA and E. coli GlnRS (QRS) or E. coli TrpRS (WRS) as described above. Cells were also co-transfected with a plasmid encoding the reporter RLucFLuc fusion protein containing the appropriate amber, ochre or opal mutation to measure suppression activity. Luciferase activity was measured as described above. Immunoblot analysis was performed as described above.

Results. As described above, we have shown that amber, ochre and opal suppressor tRNAs (hsup2 and mutants derived therefrom), derived from Escherichia coli tRNA^{Gln}, suppress UAG, UAA and UGA termination codons, respectively, in a reporter mRNA in mammalian cells. Activity of each suppressor tRNA was shown to be dependent upon the co-expression of E. coli glutaminyl-tRNA synthetases (GlnRS, QRS).

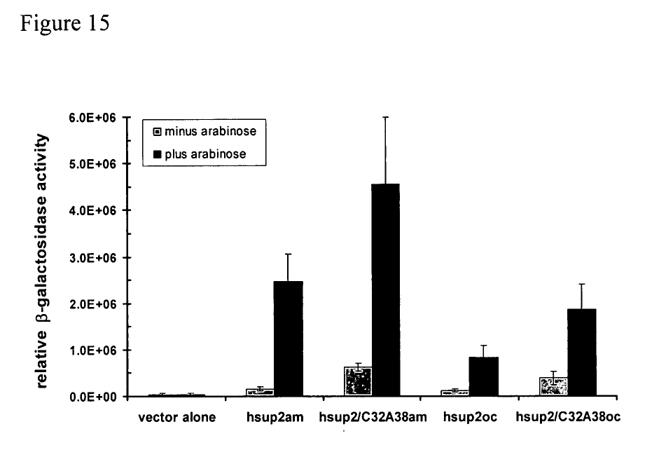

In addition, we have demonstrated that the enhanced amber and opal suppressor tRNAs derived from E. coli tRNA^{Gln} are also recognized efficiently by bacterial tryptophanyl-tRNA synthetases (TrpRS, WRS). E. coli tRNA^{Gln} and tRNA^{Trp} are closely related (FIG. 16). Both tRNAs are recognized by their cognate aminoacyl-tRNA synthetase, namely E. coli GlnRS and TrpRS, primarily through direct contacts with bases of the anticodon. Further contacts are observed with additional bases in the anticodon loop and with bases in the upper part of the acceptor stem. The enhanced amber, ochre and opal suppressor tRNAs described above, hsup2/C32A38am, hsup2/C32A38oc and hsup2/C32A38op, share features from both E. coli tRNA^{Gln} and tRNA^{Trp} (FIG. 16). The original tRNA^{Gln} acceptor stem is completely preserved, while the anticodon-loop containing the C32A38 mutations mimics that of tRNA^{Trp}. These similarities suggested to us that bacterial TrpRS might efficiently recognize the suppressor tRNAs dervied from E. coli tRNA^{Gln}.

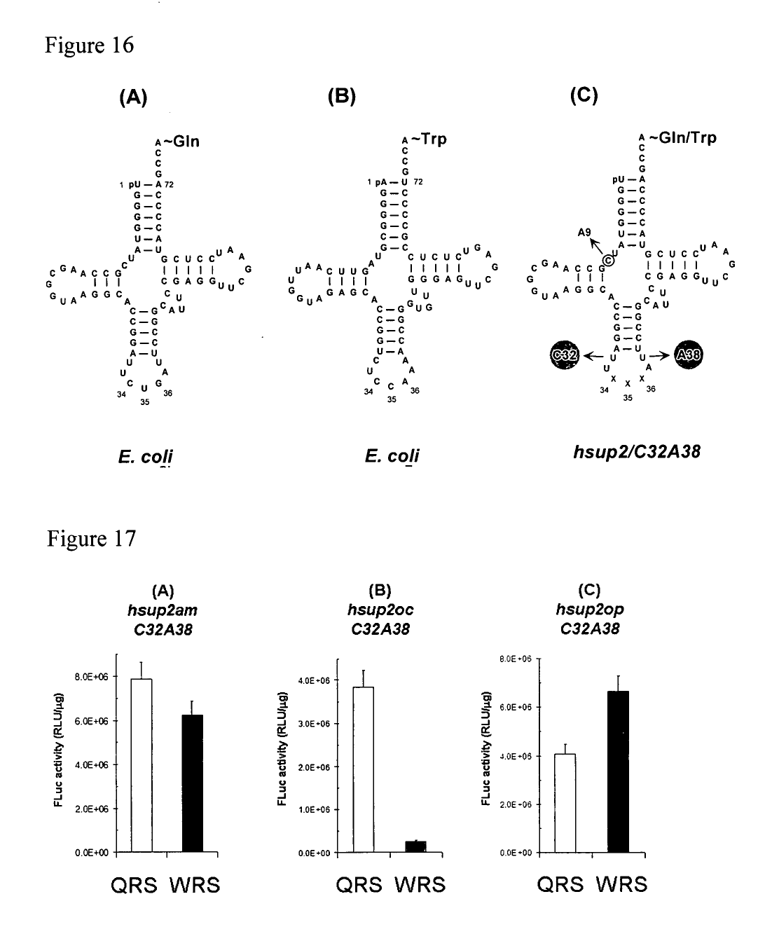

E. coli GlnRS and TrpRS were expressed alongside suppressor tRNAs hsup2/C32A38am, hsup2/C32A38oc and hsup2/C32A38op in mammalian cells that had been transfected with a plasmid encoding the reporter RLucFLuc fusion protein containing the appropriate amber, ochre or opal mutation. tRNAs hsup2/C32A38am and hsup2/C32A38op show similar activity in the presence of E. coli GlnRS and E. coli TrpRS (FIG. 17A and 17C). In contrast, the ochre suppressor tRNA hsup2/C32A38oc is inactive in the presence of E. coli TrpRS (FIG. 17B).

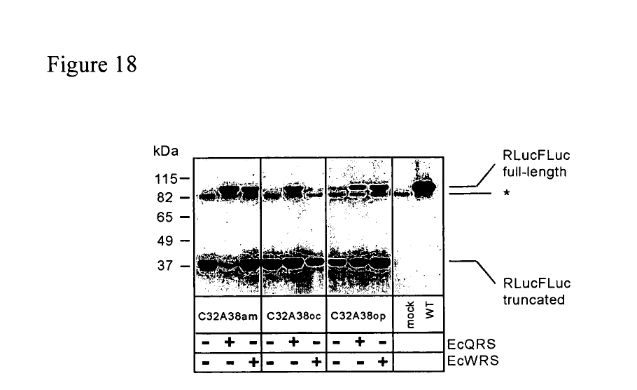

Immunoblot analysis of proteins isolated from cells co-transfected with plasmids carrying the genes encoding the luciferase reporter, hsup2/C32A38am, hsup2/C32A38oc or hsup2/C32A38op tRNA and, when present, E. coli GlnRS (EcQRS) or E. coli TrpRS (EcWRS) confirmed these results (FIG. 18). These results indicate that the same suppressor tRNAs, which we have shown to be (i) orthogonal, (ii) highly active and (iii) highly specific, may be used for incorporation of a variety of amino acid analogues, including amino acid analogs derived from glutamine or tryptophan, into proteins.

Other Embodiments, Equivalents, and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any tRNA, aminoacyl tRNA synthetase, stop codon, etc., can be excluded from any one or more claims, for any reason, whether or not related to the existence of prior art.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims:

We claim:

1. An ochre suppressor tRNA that is orthogonal to a mammalian cell.

2. The ochre suppressor of claim 1, wherein the ochre suppressor tRNA has a translation efficiency of between approximately 0.03 and approximately 4.5% when present in a mammalian cell that contains an aminoacyl-tRNA synthetase that aminoacylates the ochre suppressor tRNA.

3. The ochre suppressor of claim 1, wherein the ochre suppressor tRNA has a translation efficiency of approximately 4.5% when present in a mammalian cell that contains an aminoacyl-tRNA synthetase that aminoacylates the ochre suppressor tRNA.

4. The ochre suppressor of claim 1, wherein the ochre suppressor tRNA has a translation efficiency of at least 4.5% when present in a mammalian cell that contains an aminoacyl-tRNA synthetase that aminoacylates the ochre suppressor tRNA.

5. The ochre suppressor of claim 1, wherein the ochre suppressor is not a substrate for any native aminoacyl-tRNA synthetase in the cell.

6. The ochre suppressor tRNA of claim 1, wherein the ochre suppressor tRNA is aminoacylated with a natural or unnatural amino acid.

7. An isolated mammalian cell containing the ochre suppressor tRNA of claim 1.

8. The mammalian cell of claim 7, wherein the suppressor tRNA was not synthesized by the cell.

9. The mammalian cell of claim 7, wherein the suppressor tRNA is expressed by the cell.

10. The mammalian cell of claim 7, wherein the activity or expression of the suppressor tRNA is regulatable.

11. The mammalian cell of claim 7, wherein the ochre suppressor tRNA is not a substrate for any native aminoacyl-tRNA synthetase in the cell.

12. The mammalian cell of claim 7, further comprising an aminoacyl-tRNA synthetase that aminoacylates the ochre suppressor tRNA.

13. The mammalian cell of claim 12, wherein the aminoacyl-tRNA synthetase is orthogonal to the cell.

14. The mammalian cell of claim 12, wherein the cell expresses the aminoacyl-tRNA synthetase.

15. The mammalian cell of claim 12, wherein the cell expresses the aminoacyl-tRNA synthetase in a regulatable manner.

16. The mammalian cell of claim 12, wherein the cell expresses both the ochre suppressor tRNA and the aminoacyl-tRNA synthetase.

17. The mammalian cell of claim 12, wherein the cell further comprises a heterologous polynucleotide that comprises an open reading frame containing an ochre codon.

18. The mammalian cell of claim 17, wherein the open reading frame contains an amber codon, an opal codon, or both, and the cell further comprises an orthogonal amber suppressor tRNA-aaRS pair, an orthogonal opal suppressor tRNA-aaRS pair, or both.

19. The mammalian cell of claim 7, further comprising an amber suppressor tRNA that is orthogonal to the cell.

20. The mammalian cell of claim 19, wherein the amber suppressor tRNA has a translation efficiency of between approximately 2.8% and approximately 34% when present in a mammalian cell that contains an aminoacyl-tRNA synthetase that aminoacylates the amber suppressor tRNA.

21. The mammalian cell of claim 19, wherein the amber suppressor tRNA is not a substrate for any native aminoacyl-tRNA synthetase in the cell.

22. The mammalian cell of claim 19, further comprising an aminoacyl-tRNA synthetase that aminoacylates the ochre suppressor tRNA, the amber suppressor tRNA, or both.

23. The mammalian cell of claim 7, further comprising an opal suppressor tRNA that is orthogonal to the cell.

24. The mammalian cell of claim 23, wherein the opal suppressor is not a substrate for any native aminoacyl-tRNA synthetase in the cell.

25. The mammalian cell of claim 23, wherein the opal suppressor has a translation efficiency of between approximately 0.05% and 10% when present in a mammalian cell that contains an aminoacyl-tRNA synthetase that aminoacylates the opal suppressor tRNA.

26. The mammalian cell of claim 23, further comprising an aminoacyl-tRNA synthetase that aminoacylates the ochre suppressor tRNA, the opal suppressor tRNA, or both.

27. The mammalian cell of claim 7, further comprising an amber suppressor tRNA that is orthogonal to the cell and an opal suppressor tRNA that is orthogonal to the cell.

28. The mammalian cell of claim 27, wherein the amber suppressor tRNA, the opal suppressor tRNA, the ochre suppressor tRNA, or any combination thereof, is not a substrate for any native aminoacyl-tRNA synthetase in the cell.

29. The mammalian cell of claim 27, further comprising an aminoacyl-tRNA synthetase that aminoacylates the ochre suppressor tRNA, the amber suppressor tRNA, the opal suppressor tRNA, or any combination thereof.

30. The mammalian cell of claim 27, further comprising at least two different aminoacyl-tRNA synthetases, each of which aminoacylates at least one of the suppressor tRNAs.

31. The mammalian cell of claim 7, wherein the cell comprises (i) a heterologous polynucleotide that comprises an open reading frame containing an ochre codon and (ii) an aaRS that aminoacylates the ochre suppressor tRNA.

32. A polynucleotide that comprises a template for transcription of the ochre suppressor tRNA of claim 1, operably linked to a promoter sequence.

33. A mammalian cell comprising the polynucleotide of claim 32.

34. The mammalian cell of claim 33, further comprising a polynucleotide that comprises a template for transcription of an aaRS that aminoacylates the ochre suppressor tRNA.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. A method for synthesizing a protein in a mammalian cell by translation of genes containing at least one stop codon within the open reading frame, the method comprising steps of:
(a) providing an isolated mammalian cell containing: (i) at least one gene that includes at least one ochre codon within the open reading frame; (ii) an ochre suppressor tRNA that is orthogonal to the cell; and (iii) an aminoacyl-tRNA synthetase (aaRS) that aminoacylates the ochre suppressor tRNA; and
(b) maintaining the cell for a period of time under conditions in which protein synthesis can occur.

89. The method of claim 88, wherein the step of providing the mammalian cell comprises the step of: contacting a mammalian cell with an ochre suppressor tRNA that was not synthesized within the cell, so that the tRNA is taken up into the cell at a level sufficient to allow readthrough of the ochre codon in the cell.

90. The method of claim 88, wherein the ochre suppressor tRNA is aminoacylated with an unnatural amino acid.

91. The method of claim 88, wherein the step of providing the mammalian cell comprises the step of: introducing a polynucleotide that contains a template for transcription of the ochre suppressor tRNA into the cell, so that the cell expresses the tRNA.

92. The method of claim 88, wherein the step of providing the mammalian cell comprises the step of: introducing a polynucleotide that contains a template for transcription of the aminoacyl-tRNA synthetase into the cell, so that the cell expresses the aminoacyl-tRNA synthetase.

93. The method of claim 88, wherein the ochre suppressor tRNA is not a substrate for any native aminoacyl-tRNA synthetase in the cell.

94. The method of claim 88, wherein the gene further contains an amber or opal stop codon, or both, in the open reading frame, and wherein the cell further comprises an orthogonal amber suppressor tRNA, an orthogonal opal suppressor tRNA, or both.

95. The method of claim 88, wherein the cell is contacted with an unnatural amino acid that is taken up by the cell and used by the aaRS to aminoacylate the ochre suppressor tRNA, so that the unnatural amino acid is inserted into a growing amino acid chain at a position defined by the ochre codon in the open reading frame.

96. The method of claim 88, wherein (i) the at least one gene includes three different stop codons within the open reading frame; (ii) the cell comprises three suppressor tRNAs, wherein the suppressor tRNAs read through three different stop codons; and (iii) the cell comprises a set of one or more aminoacyl-tRNA synthetases, wherein aminoacyl-tRNA synthetases in the set of aminoacyl-tRNA synthetases aminoacylate the suppressor tRNAs.

97. A method for synthesizing a protein in a mammalian cell by translation of genes containing at least one stop codon within the open reading frame, the method comprising steps of:
(a) providing an isolated mammalian cell containing: (i) at least one gene that includes at least one ochre, amber, or opal codon within the open reading frame; (ii) one or more suppressor tRNAs selected from the group consisting of: an ochre suppressor tRNA that is orthogonal to the cell, an amber suppressor tRNA having a translation efficiency of between approximately 2.8% and approximately 34% when present in a mammalian cell that contains an aminoacyl-tRNA synthetase that aminoacylates the amber suppressor tRNA, and an opal suppressor tRNA having a translation efficiency of between approximately 0.05% and approximately 10% when present in a mammalian cell that contains an aminoacyl-tRNA synthetase that aminoacylates the opal suppressor tRNA; and (iii) one or more aminoacyl-tRNA synthetases (aaRSs) each of which aminoacylates at least one of the suppressor tRNAs; and
(b) maintaining the cell for a period of time under conditions in which protein synthesis can occur.

98. The method of claim 97, wherein the step of providing the mammalian cell comprises the step of: contacting a mammalian cell with a suppressor tRNA that was not synthesized within the cell, so that the tRNA is taken up into the cell at a level sufficient to allow readthrough of a stop codon in the cell.

99. The method of claim 97, wherein the suppressor tRNA is aminoacylated with an unnatural amino acid.

100. The method of claim 97, wherein the step of providing the mammalian cell comprises the step of: introducing a polynucleotide that contains a template for transcription of the suppressor tRNA into the cell, so that the cell expresses the tRNA.

101. The method of claim 97, wherein the step of providing the mammalian cell comprises the step of: introducing a polynucleotide that contains a template for transcription of the aminoacyl-tRNA synthetase into the cell, so that the cell expresses the aminoacyl-tRNA synthetase.

102. The method of claim 97, wherein the suppressor tRNA is not a substrate for any native aminoacyl-tRNA synthetase in the cell.

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. A kit comprising at least one suppressor tRNA that is orthogonal to a mammalian cell, or a polynucleotide or expression vector that comprises a template for synthesis of the suppressor tRNA, or both, wherein the suppressor tRNA is selected from the group consisting of: an ochre suppressor tRNA, an amber suppressor tRNA having a translation efficiency of between approximately 2.8% and approximately 34% when present in a mammalian cell that contains an aminoacyl-tRNA synthetase that aminoacylates the amber suppressor tRNA, and an opal suppressor tRNA having a translation efficiency of between approximately 0.05% and approximately 10% when present in a mammalian cell that contains an aminoacyl-tRNA synthetase that aminoacylates the opal suppressor tRNA.

119. The kit of claim 117, wherein the kit comprises an ochre suppressor tRNA that is orthogonal to a mammalian cell, or a polynucleotide or expression vector that comprises a template for synthesis of the tRNA, or both.

120. The kit of claim 119, wherein the kit further comprises one or more items selected from the group consisting of: (i) an amber suppressor tRNA that is orthogonal to a mammalian cell, or a polynucleotide or expression vector that comprises a template for synthesis of such an amber suppressor tRNA, or both; (ii) an opal suppressor tRNA that is orthogonal to a mammalian cell, or a polynucleotide or expression vector that comprises a template for synthesis of such an opal suppressor tRNA, or both.

121. The kit of claim 117, wherein the kit comprises (i) an ochre suppressor tRNA that is orthogonal to a mammalian cell, or a polynucleotide or expression vector that comprises a template for synthesis of the tRNA, or both; (ii) an amber suppressor tRNA that is orthogonal to a mammalian cell, or a polynucleotide or expression vector that comprises a template for synthesis of such an amber suppressor tRNA, or both; and (iii) an opal suppressor tRNA that is orthogonal to a mammalian cell, or a polynucleotide or expression vector that comprises a template for synthesis of such an opal suppressor tRNA, or both;

122. The kit of claim 117, further comprising at least one item selected from the group consisting of: (i) an aminoacyl-tRNA synthetases that aminoacylates a suppressor tRNA that is orthogonal to a mammalian cell, a polynucleotide or expression vector that comprises a template for synthesis of such an aminoacyl-tRNA synthetase, or both; (ii) a mammalian cell; (iii) one or more unnatural amino acids; (iv) an agent that induces or represses transcription; (v) a transfection reagent such as a lipid; (vi) an in vitro translation system; (vii) a reporter system; (viii) a buffer; (ix) tissue culture medium; and (x) instructions for use.

123. The kit of claim 117, wherein one or more of the suppressor tRNAs is not a substrate for any native aminoacyl-tRNA in the cell.

124. The kit of claim 117, wherein the kit comprises at least one collection of suppressor tRNAs selected from the group consisting of: a collection of ochre suppressor tRNAs that are orthogonal to a mammalian cell, wherein members of the collection suppress ochre codons in a mammalian cell with a range of different efficiencies; a collection of amber suppressor tRNAs that are orthogonal to a mammalian cell, wherein members of the collection suppress amber codons in a mammalian cell with a range of different efficiencies; and a collection of opal suppressor tRNAs that are orthogonal to a mammalian cell, wherein members of the collection suppress opal codons in a mammalian cell with a range of different efficiencies.

125. (canceled)

126. (canceled)

127. (canceled)

128. (canceled)

129. (canceled)

130. (canceled)

131. (canceled)

132. (canceled)

133. (canceled)
